(19) United States
(12) Patent Application Publication
(10) Pub. No.: US 2011/0301056 A1
(43) Pub. Date: Dec. 8, 2011

(54) NECTIN-4 FOR TARGET GENES OF CANCER THERAPY AND DIAGNOSIS

(75) Inventors: Yusuke Nakamura, Tokyo (JP); Yataro Daigo, Tokyo (JP); Takuya Tsunoda, Kanagawa (JP); Yoshiro Kishi, Aichi (JP); Yoshihiro Fujii, Aichi (JP)

(73) Assignee: Oncotherapy Science, Inc., Kanagawa (JP)

(21) Appl. No.: 13/133,935

(22) PCT Filed: Aug. 21, 2009
(86) PCT No.: PCT/JP2009/004026

§ 371 Date: Aug. 22, 2011

Related U.S. Application Data
(60) Provisional application No. 61/201,811, filed on Dec. 12, 2008.

(51) Int. Cl. G01N 33/574; C12Q 1/02; C40B 30/04; C12Q 1/68

(52) U.S. Cl. 506/9; 435/6.12; 435/6.11; 435/29; 435/7.23; 436/501

(57) ABSTRACT

The present invention features methods for diagnosing cancer or assessing or determining the prognosis of a patient with lung cancer, by detecting the expression level of Nectin-4. The present invention also features double-stranded molecules against the Nectin-4 gene, vectors encoding them, compositions comprising them and methods comprising the step of administering them into a subject, which are useful for treating or preventing cancer. Also, disclosed are methods of identifying candidate compounds for treating and preventing cancer, using the Nectin-4 polypeptide or cells expressing the Nectin-4 gene.

### PRIORITY

The present application claims the benefit of U.S. Provisional Application No. 61/201,811, filed on Dec. 12, 2008, the entire content of which is incorporated by reference herein.

### TECHNICAL FIELD

The present invention relates to the field of biological science, more specifically to the field of cancer research, cancer diagnosis and cancer therapy. In particular, the present invention relates to methods for detecting and diagnosing cancer as well as methods for treating and preventing lung cancer. Moreover, the present invention relates to methods for screening an agent for treating and/or preventing cancer.

### BACKGROUND ART

Non-small cell lung cancer (NSCLC) is the leading cause of cancer death worldwide (Ahmedin J, et al. 2007. CA Cancer J Clin 2007; 57:43-66). About 30% of patients who are diagnosed to have NSCLC are able to undergo curative resection, while the remaining patients with an advanced disease are mainly treated with chemotherapy alone or in combination with a local treatment modality (Parkin D M. Lancet Oncol 2001; 2:533-43). In spite of the use of modern surgical techniques combined with various adjuvant treatments, such as radiotherapy and chemotherapy, the overall 5-year survival rate of NSCLC patients still remains at only 15% (Naruke T, et al. Ann Thorac Surg 2001; 71: 1759-64, Schiller J H, et al. N Engl J Med 2002; 346:92-8). A number of targeted therapies such as bevacizumab, cetuximab, erlotinib, gefitinib, sorafenib and sunitinib were shown to be effective for a subset of advanced NSCLC patients in phase II and III trials, and some of them are already used in clinic (Thatcher N. Lung Cancer 2007; 57 Suppl 2:S18-23, Sandler A, et al. N Engl J Med 2006; 355:2542-50, Shepherd F A, et al. N Engl J Med 2005; 353:123-32, Thatcher N, et al. Lancet 2005; 366:1527-37, Cesare G, et al. The Oncologist 2007; 12:191-200). However, issues of toxicity limit these treatment regimens to selected patients. In addition, even if all kinds of available treatments are applied, the proportion of patients showing good response is still limited.

Genome-wide gene expression analysis using a microarray technology is an effective approach for identifying new molecules involved in pathways of carcinogenesis or those associated with efficacy to anti-cancer therapy; some of such genes or their gene products may be good target molecules for the development of novel therapies and/or tumor biomarkers (Daigo Y, Nakamura Y. Gen Thorac Cardiovasc Surg 2008; 56:43-53). To identify such molecules, genome-wide expression profile analysis of 101 lung cancers had been performed with the cDNA microarray containing 27,648 genes and ESTs, coupled with enrichment of tumor cells by laser microdissection (Kikuchi T, et al. Oncogene 2003; 22:2192-205, Kakiuchi S, et al. Mol Cancer Res 2003; 1:485-99, Kakiuchi S, et al. Hum Mol Genet. 2004; 13:3029-43, Kikuchi T, et al. Int J Oncol 2006; 28:799-805, Taniwaki M, et al. Int J Oncol 2006; 29:567-75). To verify the biological and clinicopathological significance of the respective gene products, tumor-tissue microarray analysis of clinical lung-cancer materials had been performed as well as RNA interference (RNAi) assays (Suzuki C, et al. Cancer Res 2003; 63:7038-41, Ishikawa N, et al. Clin Cancer Res 2004; 10:8363-70, Kato T, et al. Cancer Res 2005; 65:5638-46, Furukawa C, et al. Cancer Res 2005; 65:7102-10, Ishikawa N, et al. Cancer Res 2005; 65:9176-84, Suzuki C, et al. Cancer Res 2005; 65:11314-25, Ishikawa N, et al. Cancer Sci 2006; 97:737-45, Takahashi K, et al. Cancer Res 2006; 66:9408-19, Hayama S, et al. Cancer Res 2006; 66:10339-48, Kato T, et al. Clin Cancer Res 2007; 13:434-42, Suzuki C, et al. Mol Cancer Ther 2007; 6:542-51, Yamabuki T, et al. Cancer Res 2007; 67:2517-25, Hayama S, et al. Cancer Res 2007; 67:4113-22, Kato T, et al. Cancer Res 2007; 67:8544-53, Taniwaki M, et al. Clin Cancer Res 2007; 13:6624-31, Ishikawa N, et al. Cancer Res 2007; 67:11601-11, Mano Y, et al. Cancer Sci 2007; 98:1902-13, Suda T, et al. Cancer Sci 2007; 98:1803-8, Kato T, et al. Clin Cancer Res 2008; 14:2363-70, Mizukami Y, et al. Cancer Sci 2008; 99:1448-54, Harao M, et al. Int J Cancer 2008; 123:2616-25). This systematic approach revealed that Nectin-4 (also referred to as “PVRL 4”: poliovirus receptor-related 4) was transactivated in the non-small cell lung cancers (WO2004/031413).

The Nectin family is the Ca²⁺-independent immunoglobulin-like molecules consisting of four members (Nectin-1, -2, -3, and -4), which are thought to trans-interact homophilically and heterophilically, and play a role in cell-cell adhesion. Nectins are suggested to bind afadin, an actin filament (F-actin)-binding protein through their cytoplasmic tails and associate with the actin cytoskeleton, and could regulate many other cellular activities such as movement, differentiation, polarization, and the entry of viruses, in cooperation with other cell adhesion molecules and cell surface membrane receptors (Takai Y, et al. Nat Rev Mol Cell Biol 2008; 9:603-15). Nectin-2 and PVR interact with Nectin-3 and DNAM-1/CD226, whereas Nectin-1 interacts with Nectin-3 and Nectin-4 (Reymond N, et al. J Biol Chem 2001; 276:43205-15, Bottino C, et al. J Exp Med 2003; 198:557-67). Nectins 1, 2 and 3 are widely expressed in adult tissues, but Nectin-4 was expressed specifically in the embryo and placenta (Reymond N, et al. J Biol Chem 2001; 276:43205-15, Fabre S, et al. J Biol Chem 2002; 277:27006-13). Recently, Nectin-4 was indicated to be overexpressed in breast carcinoma (Fabre-Lafay S, et al. J Biol Chem 2005; 280:19543-50, Fabre-Lafay S, et al. BMC Cancer 2007; 7:73).

In spite of the recent evidence of Nectin-4 overexpression in cancers, the biological significance of Nectin-4 activation in human cancer progression and its clinical potential as a therapeutic target were not fully described.

### CITATION LIST

### Non Patent Literature

NPL 1: Ahmedin J, et al. 2007. CA Cancer J Clin 2007; 57:43-66
NPL 2: Parkin D M. Lancet Oncol 2001; 2:533-43
NPL 3: Naruke T, et al. Ann Thorac Surg 2001; 71: 1759-64
NPL 4: Schiller J H, et al. N Engl J Med 2002; 346:92-8
NPL 5: Thatcher N. Lung Cancer 2007; 57 Suppl 2:S18-23
NPL 6: Sandler A, et al. N Engl J Med 2006; 355:2542-50
NPL 7: Shepherd F A, et al. N Engl J Med 2005; 353:123-32
NPL 8: Thatcher N, et al. Lancet 2005; 366:1527-37
NPL 9: Cesare G, et al. The Oncologist 2007; 12:191-200
NPL 10: Daigo Y, Nakamura Y. Gen Thorac Cardiovasc Surg 2008; 56:43-53
NPL 11: Kikuchi T, et al. Oncogene 2003; 22:2192-205
NPL 12: Kakiuchi S, et al. Mol Cancer Res 2003; 1:485-99
NPL 13: Kakiuchi S, et al. Hum Mol Genet. 2004; 13:3029-43
NPL 14: Kikuchi T, et al. Int J Oncol 2006; 28:799-805
NPL 15: Taniwaki M, et al. Int J Oncol 2006; 29:567-75
NPL 16: Suzuki C, et al. Cancer Res 2003; 63:7038-41
NPL 17: Ishikawa N, et al. Clin Cancer Res 2004; 10:8363-70
NPL 18: Kato T, et al. Cancer Res 2005; 65:5638-46
NPL 19: Furukawa C, et al. Cancer Res 2005; 65:7102-10
NPL 20: Ishikawa N, et al. Cancer Res 2005; 65:9176-84
NPL 21: Suzuki C, et al. Cancer Res 2005; 65:11314-25
NPL 22: Ishikawa N, et al. Cancer Sci 2006; 97:737-45
NPL 23: Takahashi K, et al. Cancer Res 2006; 66:9408-19
NPL 24: Hayama S, et al. Cancer Res 2006; 66:10339-48
NPL 25: Kato T, et al. Clin Cancer Res 2007; 13:434-42
NPL 26: Suzuki C, et al. Mol Cancer Ther 2007; 6:542-51
NPL 27: Yamabuki T, et al. Cancer Res 2007; 67:2517-25
NPL 28: Hayama S, et al. Cancer Res 2007; 67:4113-22
NPL 29: Kato T, et al. Cancer Res 2007; 67:8544-53
NPL 30: Taniwaki M, et al. Clin Cancer Res 2007; 13:6624-31
NPL 31: Ishikawa N, et al. Cancer Res 2007; 67:11601-11
NPL 32: Mano Y, et al. Cancer Sci 2007; 98:1902-13
NPL 33: Suda T, et al. Cancer Sci 2007; 98:1803-8
NPL 34: Kato T, et al. Clin Cancer Res 2008; 14:2363-70
NPL 35: Mizukami Y, et al. Cancer Sci 2008; 99:1448-54
NPL 36: Harao M, et al. Int J Cancer 2008; 123:2616-25
NPL 37: Takai Y, et al. Nat Rev Mol Cell Biol 2008; 9:603-15
NPL 38: Reymond N, et al. J Biol Chem 2001; 276:43205-15
NPL 39: Bottino C, et al. J Exp Med 2003; 198:557-67
NPL 40: Fabre S, et al. J Biol Chem 2002; 277:27006-13
NPL 41: Fabre-Lafay S, et al. J Biol Chem 2005; 280:19543-50
NPL 42: Fabre-Lafay S, et al. BMC Cancer 2007; 7:73

### SUMMARY OF INVENTION

The present invention relates to Nectin-4, and to the roles it plays in carcinogenesis. As such, the present invention relates to novel compositions and methods for detecting, diagnosing, treating and/or preventing cancer as well as methods for screening for useful agents therefore.

In particular, the present invention provides a method for diagnosing cancer in a subject, such a method including the steps of determining the level of Nectin-4 in a subject-derived biological sample (e.g., a blood sample) and comparing this level to that found in a reference sample, typically a normal control. A high level of Nectin-4 in a sample indicates that the subject either suffers from or is at risk for developing cancer.

In a further aspect, the present invention relates to the discovery that a high expression level of Nectin-4 correlates to poor survival rate. Therefore, the present invention provides a method for assessing or determining the prognosis of a patient with cancer, which method includes the steps of detecting the expression level of Nectin-4, comparing it to a pre-determined reference expression level and determining the prognosis of the patient from the difference therebetween.

In yet another aspect, the present invention arises from the discovery that double-stranded molecules composed of specific sequences (for example, SEQ ID NOs: 10 and 11) are effective for inhibiting cellular growth of cancer cells. Specifically, double-stranded molecules (e.g., siRNAs) targeting Nectin-4 genes are provided by the present invention. These double-stranded molecules may be utilized in an isolated state or encoded in vectors and expressed from the vectors. Accordingly, it is an object of the present invention to provide such double-stranded molecules as well as vectors and host cells expressing them.

In one aspect, the present invention provides methods for inhibiting cancer cell growth and/or cellular invasion and/or treating and/or preventing lung cancer by administering the double-stranded molecules or vectors of the present invention to a subject in need thereof. Such methods encompass administering to a subject a composition composed of one or more of the double-stranded molecules or vectors.

In another aspect, the present invention provides compositions for treating and/or preventing cancer, and/or inhibiting cancer cell growth and/or cellular invasion containing at least one of the double-stranded molecules or vectors of the present invention.

In a further aspect, the present invention provides a method of screening for a candidate compound for treating and/or preventing lung cancer, and/or inhibiting cancer cell growth and/or cellular invasion. Such a candidate compound would bind with Nectin-4 polypeptides, reduce the biological activity of Nectin-4, or reduce the expression of Nectin-4 gene or reporter gene surrogating the Nectin-4 gene.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention.

### BRIEF DESCRIPTION OF DRAWINGS

Various aspects and applications of the present invention will become apparent to the skilled artisan upon consideration of the brief description of the figures and the detailed description of the present invention and its preferred embodiments that follows:

FIG. 1A-D Expression and cellular localization of Nectin-4 in lung cancers. A, top panels, Expression of Nectin-4 in 10 clinical non-small cell lung cancers (NSCLCs), examined by semi-quantitative RT-PCR. Bottom panels, Expressions of Nectin-4 in 20 lung cancer cell lines, examined by semi-quantitative RT-PCR. B, Flow cytometric analysis of the levels of endogenous Nectin-4 protein on the cell surface using anti-Nectin-4 monoclonal antibody (mAb) and four lung cancer cell lines (NCI-H2170, NCI-H358, A549 and SBC-5). Green peaks show the fluorescent intensities (Y-axis) detected by anti-Nectin-4 mAb; black peaks depict the fluorescent intensities of cells incubated with non-immunized mouse immunoglobulin (Ig) G as a negative control. Nectin-4 was detected on the cell surface in Nectin-4-expressing NCI-H2170, NCI-H358, but not in Nectin-4-non-expressing A549 and SBC-5 cells. C, Subcellular localization of endogenous Nectin-4 protein. Nectin-4 was mainly stained on the cell surface as well as cytoplasm in NCI-H2170, NCI-H358, but not in A549 and SBC-5 cells. D, Levels of secreted endogenous Nectin-4 protein detected with ELISA in culture medium from NCI-H2170, NCI-H358, A549, and SBC-5 cells.

FIG. 1E-F E, top and middle panels, Expression of Nectin-4 in 14 NSCLCs (T; 7 clinical lung adenocarcinomas and 7 clinical lung squamous cell carcinomas) and corresponding normal lung tissues (N), examined by semi-quantitative RT-PCR. Bottom panels, Expressions of Nectin-4 in 20 lung cancer cell lines and normal bronchial epithelial cell, examined by semi-quantitative RT-PCR. F, Immunoprecipitation analysis in COS-7 cells transfected with Nectin-4 expression vector (left panel) and mock-transformant cells (right panel) by monoclonal antibodies to Nectin-4 (clones 19-33 and 66-97). These transformant cells were immunoprecipitated by either of two monoclonal antibodies to Nectin-4 (clones 19-33 and 66-97) and immunoblotted with anti-myc antibodies.

FIG. 2A Expression of Nectin-4 in normal tissues and lung tumors, and its association with poor prognosis for NSCLC patients. A, Immunohistochemical evaluation of Nectin-4 protein in representative lung adenocarcinoma (ADC) and six normal tissues; heart, lung, liver, kidney, trachea, and placenta (original magnification×100).

FIG. 2B B, top panels, Immunohistochemical staining of Nectin-4 protein in representative lung ADCs using anti-Nectin-4 antibody on tissue microarrays. Examples of strong, weak, and absent Nectin-4 expression in lung ADCs (original magnification×100). Middle panel, Immunohistochemical staining of Nectin-4 protein using anti-Nectin-4 antibody (clone 19-33) in eight representative lung tumors and adjacent normal lung tissues (original magnification×100). Bottom panel, Kaplan-Meier analysis of survival in patients with NSCLC according to Nectin-4 expression (P<0.0001; Log-rank test).

FIG. 3A-B Serologic concentration of Nectin-4 in NSCLC patients, and its association with poor prognosis for advanced NSCLC patients. Serologic concentration of Nectin-4 protein determined by ELISA in patients with NSCLC and in healthy volunteers or non-neoplatic lung disease patients with COPD. A, left panels, Serum Nectin-4 levels (units/ml) from patients with lung ADC or lung SCC. Differences were significant between ADC patients and healthy volunteers (P<0.0001 by Mann-Whitney U test) and between SCC patients and healthy volunteers (P<0.0001 by Mann-Whitney U test). The difference between healthy volunteers and benign lung disease with COPD was not significant (indicated as N.S.). Right panels, Distribution of Nectin-4 in sera from patients at various clinical stages of lung cancer. B, left panels, receiver-operating characteristic (ROC) curve analysis of Nectin-4 (red), CEA (yellow) and CYFRA21-1 (green) as serum tumor biomarkers for NSCLC (X-axis, 1-specificity; Y-axis, sensitivity). Right panels, Serum Nectin-4 levels before and after surgery (postoperative days at two months) in patients with NSCLC.

FIG. 3C-D C, Serum Nectin-4 levels (units/ml) and the expression levels of Nectin-4 in primary tumor tissues in the same NSCLC patients. Score indicates the intensity of Nectin-4 staining that was evaluated using the criteria described in Materials and Methods (original magnification×100). D, Kaplan-Meier analysis of survival after the 1st-line standard chemotherapy with two drugs combination in 88 patients with advanced NSCLC (stage IIIB-IV) who were newly diagnosed and previously untreated, according to serum Nectin-4 positivity at diagnosis (P=0.0042; Log-rank test).

FIG. 3E E, top panel, Kaplan-Meier analysis of survival after surgery in 95 patients with stage I NSCLC, according to serum Nectin-4 positivity. Bottom panel, Kaplan-Meier analysis of survival after the 1st-line chemotherapy with a combination of two drugs (carboplatin and paclitaxel) in 62 patients with stage IIIB-IV NSCLC, according to serum Nectin-4 positivity.

FIG. 4A-B Inhibition of growth of NSCLC cells by siRNA against Nectin-4. A, top panels, Expression of Nectin-4 in NCI-H2170 and NCI-H358 cells treated with si-Nectin-4-#1, si-Nectin-4-#2 or control siRNAs (si-LUC and si-CNT) analyzed by semi-quantitative RT-PCR. Middle panels, The image of colony-formation assays of NCI-H2170 and NCI-H358 cells transfected with Nectin-4-specific siRNAs or control siRNAs. Bottom panels, MTT assay of NCI-H2170 and NCI-H358 cells transfected with Nectin-4-specific siRNAs or control siRNAs. All assays were performed three times, and in triplicate wells. B, top panels, Transient expression of Nectin-4 in COS-7 and NIH-3T3 cells that were detected by western blot analysis. Immunoblotting was performed with anti-myc antibodies for myc/His-tagged Nectin-4 detection or anti-ACTB antibodies. Middle and bottom panels, Assays demonstrating the invasive nature of NIH-3T3 and COS-7 cells in Matrigel matrix after transfection with expression plasmids for human Nectin-4. Giemsa staining (middle panels; magnification, ×100), and the relative number of cells migrating through the Matrigel-coated filters (bottom panels). Assays were done thrices and in triplicate wells.

FIG. 4C-D C, Subcellular localization of exogenous Nectin-4 and F-actin in COS-7 (top panels) and NIH-3T3 (bottom panels) cells, detected with Alexa488-conjugated secondary antibodies to detect myc/His-tagged Nectin-4 and Alexa594-conjugated phalloidin, 48 hours after transient transfection of Nectin-4-expressing plasmids. D, Effect of Nectin-4 overexpression on the activation of Rac1. COS-7 (left panels) and NIH-3T3 (right panels) cells were transfected with myc/His-tagged Nectin-4 expressing plasmid or mock vector. Immunoblotting was performed with anti-Rac1, anti-myc for myc/His-tagged Nectin-4, or anti-ACTB antibodies (bottom three panels). Aliquots of cell lysates were incubated with PAK-RBD agarose beads, and subjected to a pull-down assay and subsequent immunoblotting with anti-Rac1 (top panels).

FIG. 4E-F E, In vitro enhanced growth of PC-14 cells stably expressing exogenous Nectin-4. Top panels, Expression of Nectin-4 protein in PC-14 cells transfected with a Myc/His-tagged Nectin-4 expression vector or a mock vector, examined by Western blotting. Bottom panel, MTT assay of PC-14 cells transfected with a Myc/His-tagged Nectin-4 expression vector or a mock vector. F, In vivo rapid growth of PC-14 cells stably expressing Nectin-4. Top panels, Expression of Nectin-4 protein in PC-14 cells injected subcutaneously into the posterior dorsum of BALB/cAJcl-nu/nu mice, detected by western blot analysis at 20 days after cell transplantation. Middle panels, Immunohistochemical evaluation of Nectin-4 expression in transplanted tumors at 20 days after cell transplantation (original magnification×200). Bottom panel, Tumor growth curves in nude mice after inoculation of PC-14-Nectin-4#B cells (indexed as circle; n=3 mice) or PC-14-Mock-#B cells (indexed by square; n=3). Mean tumor volume+/−1SE was plotted.

### DESCRIPTION OF EMBODIMENTS

Although methods and materials similar or equivalent to those described herein can be used in the practice or testing of embodiments of the present invention, the preferred methods and materials are now described. However, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the present invention which will be limited only by the appended claims.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the present invention, the following definitions apply:

### DEFINITIONS

The words “a”, “an”, and “the” as used herein mean “at least one” unless otherwise specifically indicated.

The term “polynucleotide”, “oligonucleotide” “nucleotide”, “nucleic acid”, and “nucleic acid molecule” are used interchangeably herein to refer to a polymer of nucleic acid residues and, unless otherwise specifically indicated are referred to by their commonly accepted single-letter codes. The terms apply to nucleic acid (nucleotide) polymers in which one or more nucleic acids are linked by ester bonding. The nucleic acid polymers may be composed of DNA, RNA or a combination thereof and encompass both naturally-occurring and non-naturally occurring nucleic acid polymers.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

Nectin-4 Gene:

The nucleic acid and polypeptide sequences of Nectin-4 in the present invention are known to those skilled in the art, and obtained, for example, from gene databases on the web site such as GenBank™. An exemplified nucleic acid sequence of the Nectin-4 gene is shown in SEQ ID NO: 1 (GenBank accession No. NM_{—}030916), and an exemplified amino acid sequence of the Nectin-4 polypeptide is shown in SEQ ID NO: 2 (GenBank accession No. NP₁₃ 112178.2). One of skill will recognize that Nectin-4 sequences need not be limited to these sequences and that variants (e.g., functional equivalents and allelic variants) can be used in the present invention as described below.

According to an aspect of the present invention, functional equivalents are also considered to be above “polypeptide”. Herein, a “functional equivalent” of a polypeptide is a polypeptide that has a biological activity equivalent to the polypeptide. Namely, any polypeptide that retains the biological ability may be used as such a functional equivalent in the present invention. The polypeptide in the present invention may have variations in amino acid sequence, molecular weight, isoelectric point, the presence or absence of sugar chains, or form, depending on the cell or host used to produce it or the purification method utilized.

Such functional equivalents include those wherein one or more amino acids are substituted, deleted, added, or inserted to the natural occurring amino acid sequence of the protein. Alternatively, the polypeptide may be composed an amino acid sequence having at least about 80% homology (also referred to as sequence identity) to the sequence of the respective protein, more preferably at least about 90% to 95% homology. In other embodiments, the polypeptide can be encoded by a polynucleotide that hybridizes under stringent conditions to the natural occurring nucleotide sequence of the gene.

The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10 degrees C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times of background, preferably 10 times of background hybridization. Exemplary stringent hybridization conditions include the following: 50% formamide, 5×SSC, and 1% SDS, incubating at 42 degrees C., or, 5×SSC, 1% SDS, incubating at 65 degrees C., with wash in 0.2×SSC, and 0.1% SDS at 50 degrees C.

In the context of the present invention, a condition of hybridization for isolating a DNA encoding a polypeptide functionally equivalent to the above human protein can be routinely selected by a person skilled in the art. For example, hybridization may be performed by conducting pre-hybridization at 68 degrees C. for 30 min or longer using “Rapid-hyb buffer” (Amersham LIFE SCIENCE), adding a labeled probe, and warming at 68 degrees C. for 1 hour or longer. The following washing step can be conducted, for example, in a low stringent condition. An exemplary low stringent condition may include 42 degrees C., 2×SSC, 0.1% SDS, preferably 50 degrees C., 2×SSC, 0.1% SDS. High stringency conditions are often preferably used. An exemplary high stringency condition may include washing 3 times in 2×SSC, 0.01% SDS at room temperature for 20 min, then washing 3 times in 1×SSC, 0.1% SDS at 37 degrees C. for 20 min, and washing twice in 1×SSC, 0.1% SDS at 50 degrees C. for 20 min. However, several factors, such as temperature and salt concentration, can influence the stringency of hybridization and one skilled in the art can suitably select the factors to achieve the requisite stringency.

Generally, it is known that modifications of one or more amino acid in a protein do not influence the function of the protein. In fact, mutated or modified proteins, proteins having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence which alter a single amino acid or a small percentage of amino acids or those considered to be a “conservative modifications”, wherein the alteration of a protein results in a protein with similar functions, are acceptable in the context of the instant invention.

So long as the activity the protein is maintained, the number of amino acid mutations is not particularly limited. However, it is generally preferred to alter 5% or less of the amino acid sequence. Accordingly, in a preferred embodiment, the number of amino acids to be mutated in such a mutant is generally 30 amino acids or less, preferably 20 amino acids or less, more preferably 10 amino acids or less, more preferably 6 amino acids or less, and even more preferably 3 amino acids or less.

An amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Conservative substitution tables providing functionally similar amino acids are well known in the art. For example, the following eight groups each contain amino acids that are conservative substitutions for one another:
1) Alanine (A), Glycine (G);
2) Aspartic acid (D), Glutamic acid (E);
3) Aspargine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins 1984).

Such conservatively modified polypeptides are included in the present protein. However, the present invention is not restricted thereto and the protein includes non-conservative modifications, so long as at least one biological activity of the protein is retained. Furthermore, the modified proteins do not exclude polymorphic variants, interspecies homologues, and those encoded by alleles of these proteins.

Moreover, the gene of the present invention encompasses polynucleotides that encode such functional equivalents of the protein. In addition to hybridization, a gene amplification method, for example, the polymerase chain reaction (PCR) method, can be utilized to isolate a polynucleotide encoding a polypeptide functionally equivalent to the protein, using a primer synthesized based on the sequence above information. Polynucleotides and polypeptides that are functionally equivalent to the human gene and protein, respectively, normally have a high homology to the originating nucleotide or amino acid sequence. “High homology” typically refers to a homology of 40% or higher, preferably 60% or higher, more preferably 80% or higher, even more preferably 90% to 95% or higher. The homology of a particular polynucleotide or polypeptide can be determined by following the algorithm in “Wilbur and Lipman, Proc Natl Acad Sci USA 80: 726-30 (1983)”.

A Method for Diagnosing Cancer:

The present invention provides a method for diagnosing cancer by determining the expression level of Nectin-4 in the subject. In the context of the present invention, any cancers, which is related to Nectin-4 overexpression, may be diagnosed, and cancer to be diagnosed is preferably lung cancer, bladder cancer or cervical carcinomas, more preferably lung cancer, further more preferably non-small cell lung cancer (NSCLC), including lung adenocarcinoma (ADC) and lung squamous cell carcinoma (SCC).

According to the present invention, an intermediate result for examining the condition of a subject may be provided. Such intermediate result may be combined with additional information to assist a doctor, nurse, or other practitioner to determine that a subject suffers from the disease. That is, the present invention provides a diagnostic marker Nectin-4 for examining cancer. Alternatively, the present invention provides a method for detecting or identifying cancer cells in a subject-derived lung bladder or cervical tissue sample, said method including the step of determining the expression level of the Nectin-4 gene in a subject-derived biological sample, wherein an increase in said expression level as compared to a normal control level of said gene indicates the presence or suspicion of cancer cells in the tissue.

Such result may be combined with additional information to assist a doctor, nurse, or other healthcare practitioner in diagnosing a subject as afflicted with the disease. In other words, the present invention may provide a doctor with useful information to diagnose a subject as afflicted with the disease. For example, according to the present invention, when there is doubt regarding the presence of cancer cells in the tissue obtained from a subject, clinical decisions can be reached by considering the expression level of the Nectin-4 gene, plus a different aspect of the disease including tissue pathology, levels of known tumor marker(s) in blood, and clinical course of the subject, etc. For example, some well-known diagnostic lung tumor markers in blood are TAP, ACT, BFP, CA19-9, CA50, CA72-4, CA130, CEA, KMO-1, NSE, SCC, SP1, Span-1, TPA, CSLEX, SLX, STN and CYFRA. Alternatively, diagnostic bladder tumor markers in blood such as TAP, SCC, and TPA are also well known. Alternatively, diagnostic cervical tumor markers in blood such as CA130, CEA, TAP, SCC, SLX, and TPA are also well known. Namely, in this particular embodiment of the present invention, the outcome of the gene expression analysis serves as an intermediate result for further diagnosis of a subject's disease state.

Specifically, the present invention provides the following methods [1] to [10]:

[1] A method for diagnosing lung cancer, said method including the steps of:

(a) detecting the expression level of the gene encoding the amino acid sequence of Nectin-4 in a biological sample; and

(b) correlating an increase in the expression level detected as compared to a normal control level of the gene to the presence of disease.

[2]The method of [1], wherein the expression level is at least 10% greater than the normal control level.

[3]The method of [1], wherein the expression level is detected by methods selected from among:

(a) detecting an mRNA including the sequence of Nectin-4,

(b) detecting a protein including the amino acid sequence of Nectin-4, and

(c) detecting a biological activity of a protein including the amino acid sequence of Nectin-4.

[4]The method of [1], wherein the lung cancer is NSCLC.

[5]The method of [3], wherein the expression level is determined by detecting hybridization of a probe to a gene transcript of the gene.

[6]The method of [3], wherein the expression level is determined by detecting the binding of an antibody against the protein encoded by a gene as the expression level of the gene.

[7]The method of [1], wherein the biological sample includes biopsy specimen, sputum or blood.

[8]The method of [1], wherein the subject-derived biological sample includes a lung tissue.

[9]The method of [1], wherein the subject-derived biological sample includes an epithelial cell.

[10]The method of [1], wherein the subject-derived biological sample includes a cancerous epithelial cell.

The method of diagnosing lung cancer will be described in more detail below.

As used herein, the term “biological sample” refers to a whole organism or a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). “Biological sample” further refers to a homogenate, lysate, extract, cell culture or tissue culture prepared from a whole organism or a subset of its cells, tissues or component parts, or a fraction or portion thereof. Lastly, “biological sample” refers to a medium, such as a nutrient broth or gel in which an organism has been propagated, which contains cellular components, such as proteins or polynucleotides.

A subject to be diagnosed by the present method is preferably a mammal. Exemplary mammals include, but are not limited to, e.g., human, non-human primate, mouse, rat, dog, cat, horse, and cow.

It is preferred to collect a biological sample from the subject to be diagnosed to perform the diagnosis. Any biological material can be used as the biological sample for the determination so long as it includes the objective transcription or translation product of Nectin-4. The biological samples include, but are not limited to, biopsy specimen, bodily tissues and fluids, such as blood, sputum and urine. Preferably, the biological sample contains a cell population comprising an epithelial cell, more preferably a cancerous epithelial cell or an epithelial cell derived from tissue suspected to be cancerous. Further, if necessary, the cell may be purified from the obtained bodily tissues and fluids, and then used as the biological sample.

According to the present invention, the expression level of Nectin-4 in the subject-derived biological sample is determined. The expression level can be determined at the transcription (nucleic acid) product level, using methods known in the art. For example, the mRNA of Nectin-4 may be quantified using probes by hybridization methods (e.g., Northern hybridization). The detection may be carried out on a chip or an array. The use of an array is preferable for detecting the expression level of a plurality of genes (e.g., various cancer specific genes) including Nectin-4. Those skilled in the art can prepare such probes utilizing the sequence information of the Nectin-4 (SEQ ID NO 1; GenBank accession number: NM_{—}030916). For example, the cDNA of Nectin-4 may be used as the probes. If necessary, the probe may be labeled with a suitable label, such as dyes, fluorescent and isotopes, and the expression level of the gene may be detected as the intensity of the hybridized labels.

Furthermore, the transcription product of Nectin-4 may be quantified using primers by amplification-based detection methods (e.g., RT-PCR). Such primers can also be prepared based on the available sequence information of the gene. For example, the primers (SEQ ID NO 3 and 4) used in the Example may be employed for the detection by RT-PCR or Northern blot, but the present invention is not restricted thereto.

Specifically, a probe or primer used for the present method hybridizes under stringent, moderately stringent, or low stringent conditions to the mRNA of Nectin-4. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degrees C. lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degrees C. for short probes or primers (e.g., 10 to 50 nucleotides) and at least about 60 degrees C. for longer probes or primers. Stringent conditions may also be achieved with the addition of destabilizing agents, such as formamide.

Alternatively, the translation product may be detected for the diagnosis of the present invention. For example, the quantity of Nectin-4 protein may be determined. A method for determining the quantity of the protein as the translation product includes immunoassay methods that use an antibody specifically recognizing the protein. The antibody may be monoclonal or polyclonal. Furthermore, any fragment or modification (e.g., chimeric antibody, scFv, Fab, F(ab′)2, Fv, etc.) of the antibody may be used for the detection, so long as the fragment retains the binding ability to Nectin-4 protein. Methods to prepare these kinds of antibodies for the detection of proteins are well known in the art, and any method may be employed in the present invention to prepare such antibodies and equivalents thereof.

As another method to detect the expression level of Nectin-4 gene based on its translation product, the intensity of staining may be observed via immunohistochemical analysis using an antibody against Nectin-4 protein. Namely, the observation of strong staining indicates increased presence of the protein and at the same time high expression level of Nectin-4 gene.

Moreover, in addition to the expression level of Nectin-4 gene, the expression level of other cancer-associated genes, for example, genes known to be differentially expressed in lung cancer may also be determined to improve the accuracy of the diagnosis.

The expression level of cancer marker gene including Nectin-4 gene in a biological sample can be considered to be increased if it increases from the control level of the corresponding cancer marker gene by, for example, 10%, 25%, or 50%; or increases to more than 1.1 fold, more than 1.5 fold, more than 2.0 fold, more than 5.0 fold, more than 10.0 fold, or more.

The control level may be determined at the same time with the test biological sample by using a sample(s) previously collected and stored from a subject/subjects whose disease state (cancerous or non-cancerous) is/are known. Alternatively, the control level may be determined by a statistical method based on the results obtained by analyzing previously determined expression level(s) of Nectin-4 gene in samples from subjects whose disease state are known. Furthermore, the control level can be a database of expression patterns from previously tested cells. Moreover, according to an aspect of the present invention, the expression level of Nectin-4 gene in a biological sample may be compared to multiple control levels, which control levels are determined from multiple reference samples. It is preferred to use a control level determined from a reference sample derived from a tissue type similar to that of the patient-derived biological sample. Moreover, it is preferred, to use the standard value of the expression levels of Nectin-4 gene in a population with a known disease state. The standard value may be obtained by any method known in the art. For example, a range of mean+/−2 S.D. or mean+/−3 S.D. may be used as standard value.

In the context of the present invention, a control level determined from a biological sample that is known not to be cancerous is referred to as a “normal control level”. On the other hand, if the control level is determined from a cancerous biological sample, it is referred to as a “cancerous control level”.

When the expression level of Nectin-4 gene is increased as compared to the normal control level or is similar to the cancerous control level, the subject may be diagnosed to be suffering from or at a risk of developing cancer. Furthermore, in the case where the expression levels of multiple cancer-related genes are compared, a similarity in the gene expression pattern between the sample and the reference which is cancerous indicates that the subject is suffering from or at a risk of developing cancer.

Difference between the expression levels of a test biological sample and the control level can be normalized to the expression level of control nucleic acids, e.g., housekeeping genes, whose expression levels are known not to differ depending on the cancerous or non-cancerous state of the cell. Exemplary control genes include, but are not limited to, beta-actin, glyceraldehyde 3 phosphate dehydrogenase, and ribosomal protein P1.

Serological Diagnosis of Cancer:

The present invention also provides Nectin-4 as a novel serological cancer marker. Namely, by measuring the level of Nectin-4 in subject-derived blood samples, the occurrence of or a predisposition to develop cancer expressing Nectin-4 in a subject can be determined.

In the context of the present invention, any cancers, which is related to Nectin-4 overexpression, may be diagnosed, and cancer to be diagnosed is preferably lung cancer, bladder cancer or cervical carcinomas, more preferably lung cancer, further more preferably non-small cell lung cancer (NSCLC), including lung adenocarcinoma (ADC) and lung squamous cell carcinoma (SCC).

Accordingly, the present invention involves determining (e.g., measuring) the level of Nectin-4 in blood samples. In the present invention, a method for diagnosing cancer also includes a method for testing or detecting cancer. Alternatively, in the present invention, diagnosing cancer also refers to showing a suspicion, risk, or possibility of cancer in a subject, or using Nectin-4 as a cancer marker.

Alternatively, by measuring the level of Nectin-4 in subject-derived blood samples, the occurrence of or a predisposition to develop cancer expressing Nectin-4 in a subject can be determined. Accordingly, the present invention involves determining (e.g., measuring) the level of Nectin-4 in blood samples. In the present invention, a method for diagnosing cancer also includes a method for testing or detecting cancer. Alternatively, in the present invention, diagnosing cancer also refers to showing a suspicion, risk, or possibility of cancer in a subject.

Any blood samples may be used for determining the level of Nectin-4 so long as Nectin-4 can be detected in the samples. Preferably, the blood samples include whole blood, serum, and plasma, more preferably serum.

In the present invention, the “level of Nectin-4 in blood samples” refers to the concentration of Nectin-4 present in the blood after correcting the corpuscular volume in the whole blood. One of skilled in the art will recognize that the percentage of corpuscular volume in the blood varies greatly between individuals. For example, the percentage of erythrocytes in the whole blood is very different between men and women. Furthermore, differences between individuals cannot be ignored. Therefore, the apparent concentration of a substance in the whole blood which includes corpuscular components varies greatly depending on the percentage of corpuscular volume. For example, even if the concentration in the serum is the same, the measured value for a sample with a large amount of corpuscular component will be lower than the value for a sample with a small amount of corpuscular component. Therefore, to compare the measured values of components in the blood, values for which the corpuscular volume has been corrected are usually used.

For example, by measuring components in the blood using, as samples, serum or plasma obtained by separating blood cells from the whole blood, measured values from which the effect from the corpuscular volume has been removed can be obtained. Therefore, the level of Nectin-4 in the present invention can usually be determined as a concentration in the serum or plasma. Alternatively, it may first be measured as a concentration in the whole blood, and then the effect from the corpuscular volume may be corrected. Methods for measuring a corpuscular volume in a whole blood sample are known.

Subjects diagnosed for cancer according to the present methods are preferably mammals and include humans, non-human primates, mice, rats, dogs, cats, horses and cows. A preferable subject of the present invention is a human.

In the present invention, a subject may be a patient suspected of having cancer or healthy individuals. The patient may be diagnosed by the present invention to facilitate clinical decision-making. In another embodiment, the present invention may also be applied to healthy individuals for screening of cancer.

Furthermore, an intermediate result for examining the condition of a subject may be provided. Such intermediate result may be combined with additional information to assist a doctor, nurse, or other practitioner to diagnose that a subject suffers from the disease. Alternatively, the present invention may be used to detect Nectin-4 as a cancer marker in a blood sample, and provide a doctor with useful information to diagnose that the subject, from which the blood sample is derived, suffers from the disease. In other word, the present invention may provide a serological cancer marker for determined a blood sample derived from a subject who has cancerous cells.

In one embodiment of the present invention, the level of Nectin-4 is determined by measuring the quantity or concentration of Nectin-4 protein in blood samples. Methods for determining the quantity of the Nectin-4 protein in blood samples include immunoassay methods. The immunoassay methods may be preferably ELISA, and antibodies to be used for the immunoassay methods may be preferably antibodies raised against the ectodomain of Nectin-4.

In the methods of diagnosis of the present invention, the blood concentration of CEA or CYFRA 21-1 may be determined, in addition to the blood concentration of Nectin-4, to detect cancer. Therefore, the present invention provides methods for diagnosing cancer, in which cancer is detected when either the blood concentration of Nectin-4 or the blood concentration of CEA or CYFRA 21-1, or both of them, are higher as compared with healthy individuals.

Carcinoembryonic antigen (CEA) is a frequently studied tumor marker of cancer including lung cancer.

Cytokeratin 19-fragment (CYFRA 21-1) is a useful marker in lung carcinomas especially, non-small sell lung cancer (NSCLC). In the present description CYFRA 21-1 is shown as CYFRA. As described above, CEA or CYFRA has already been used as serological marker for diagnosing or detecting lung cancer. However, the sensitivity of CEA or CYFRA as a marker for lung cancer is somewhat insufficient for detecting lung cancer, completely. Accordingly, it is required that the sensitivity of diagnosing lung cancer would be improved.

In the present invention, a novel serological marker for lung cancer, Nectin-4, is provided. As shown in Examples discussed bellow, Nectin-4 shows higher sensitivity than these conventional serological markers, CEA and CYFRA, and therefore, improvement in the sensitivity of diagnostic or detection methods for lung cancer may be achieved by the present invention. Namely, the present invention provides a method for diagnosing cancer in a subject, including the steps of:

(a) collecting a blood sample from a subject to be diagnosed;

(b) determining a level of Nectin-4 in the blood sample;

(c) comparing the Nectin-4 level determined in step (b) with that of a normal control, wherein a high Nectin-4 level in the blood sample, as compared to the normal control, indicates that the subject suffers from lung cancer

In another embodiment, the method of the present invention may further include the steps of:

(d) determining a level of CEA in the blood sample;

(e) comparing the CEA level determined in step (d) with that of a normal control; and

(f) judging that the subject suffers from cancer, when the level of Nectin-4 and/or the level of the CEA are higher than the control levels.

By the combination between Nectin-4 and CEA, the sensitivity for detection of cancer, especially lung cancer may be significantly improved. In more particularly, the combination of Nectin-4 and CEA is preferably applied to NSCLCs, more preferably, adenocarcinomas (ADCs). For example, in the group analyzed in the working example discussed below, positive rate of Nectin-4 and CEA for lung cancer is about 54.5% and 42.3%, respectively. In comparison, that of combination between CEA and Nectin-4 increases to 65.0%. In the present invention, “combination of CEA and Nectin-4” refers to either or both levels of CEA and Nectin-4 being used as marker. In the preferable embodiments, a patient with positive either of CEA or Nectin-4 may be judged to have a high risk of cancer. The use of combination of Nectin-4 and CEA as serological marker for cancer is novel.

In another embodiment, the present invention may further include the steps of:

(d) determining a level of CYFRA in the blood sample;

(e) comparing the CYFRA level determined in step (d) with that of a normal control; and

(f) judging that the subject suffers from cancer, when the level of Nectin-4 and/or the level of the CYFRA are higher than the control levels.

By combining Nectin-4 and CYFRA, the sensitivity for detection of cancer, especially lung cancer, may be significantly improved. In more particularly, the combination of Nectin-4 and CYFR is preferably applied to NSCLCs, more preferably, squamous-cell carcinomas (SCCs). For example, in the group analyzed in the working example discussed below, positive rate of Nectin-4 and CYFRA for lung cancer is about 51.2% and 53.7%, respectively. In comparison, that of combination between CYFRA and Nectin-4 increases to 68.3%. In the present invention, “combination of CYFRA and Nectin-4” refers to either or both levels of CYFRA and Nectin-4 being used as marker. In the preferable embodiments, a patient with positive either of CYFRA and Nectin-4 may be judged to have a high risk of lung cancer. The use of combination of Nectin-4 and CYFRA as serological marker for lung cancer is a novel discovery of the present invention.

Therefore, the present invention can greatly improve the sensitivity for detecting cancer patients, compared to determinations based on results of measuring CEA or CYFRA alone. Behind this improvement is the fact that the group of CEA- or CYFRA-positive patients and the group of Nectin-4-positive patients do not match completely.

For example, among patients who, as a result of CEA or CYFRA measurements, were determined to have a lower value than a standard value (i.e., not to have cancer), there is actually a certain percentage of patients that have cancer. Such patients are referred to as CEA- or CYFRA-false negative patients. By combining a determination based on CEA or CYFRA with a determination based on Nectin-4, patients whose Nectin-4 value is above the standard value can be found from among the CEA- or CYFRA-false-negative patients. That is, from among patients falsely determined to be “negative” due to a low blood concentration of CEA or CYFRA, the present invention provides a means to identify patients actually having cancer. The sensitivity for detecting cancer patients is thus improved by the present invention. Generally, simply combining the results from determinations using multiple markers may increase the detection sensitivity, but on the other hand, it often causes a decrease in specificity. However, by determining the best balance between sensitivity and specificity, the present invention has determined a characteristic combination that can increase the detection sensitivity without compromising the specificity.

In the present invention, in order to consider the results of CEA or CYFRA measurements at the same time, for example, the blood concentration of CEA or CYFRA may be measured and compared with standard values, in the same way as for the aforementioned comparison between the measured values and standard values of Nectin-4. For example, how to measure the blood concentration of CEA or CYFRA and compare it to standard values are already known. Moreover, ELISA kits for CEA or CYFRA are also commercially available. These methods described in known reports can be used in the method of the present invention for diagnosing or detecting cancer.

In the present invention, the standard value of the blood concentration of Nectin-4 can be determined statistically. For example, the blood concentration of Nectin-4 in healthy individuals can be measured to determine the standard blood concentration of Nectin-4 statistically. When a statistically sufficient population is gathered, a value in the range of twice or three times the standard deviation (S.D.) from the mean value is often used as the standard value. Therefore, values corresponding to the mean value+2×S.D. or mean value+3×S.D. may be used as standard values. The standard values set as described theoretically comprise 90% and 99.7% of healthy individuals, respectively.

Alternatively, standard values can also be set based on the actual blood concentration of Nectin-4 in cancer patients. Generally, standard values set this way minimize the percentage of false positives, and are selected from a range of values satisfying conditions that can maximize detection sensitivity. In this case, the standard values are usually referred to as “cut off value”. Herein, the percentage of false positives refers to a percentage, among healthy individuals, of patients whose blood concentration of Nectin-4 is judged to be higher than a standard value (cut off value). On the contrary, the percentage, among healthy individuals, of patients whose blood concentration of Nectin-4 is judged to be lower than a standard value (cut off value) indicates specificity. That is, the sum of the false positive percentage and the specificity is always 1. The detection sensitivity refers to the percentage of patients whose blood concentration of Nectin-4 is judged to be higher than a standard value (cut off value), among all cancer patients within a population of individuals for whom the presence of cancer has been determined.

Furthermore, in the present invention, the percentage of cancer patients among patients whose Nectin-4 concentration was judged to be higher than a standard value (cut off value) represents the positive predictive value. On the other hand, the percentage of healthy individuals among patients whose Nectin-4 concentration was judged to be lower than a standard value (cut off value) represents the negative predictive value. The relationship between these values is summarized in Table 1. As the relationship shown below indicates, each of the values for sensitivity, specificity, positive predictive value, and negative predictive value, which are indexes for evaluating the diagnostic accuracy for lung cancer, varies depending on the standard value (cut off value) for judging the level of the blood concentration of Nectin-4.

TABLE 1

| Blood concentration | Lung cancer | Healthy | |
|---|---|---|---|
| of Nectin-4 | patients | individuals | |
| High | a: True | b: False | Positive predictive |
| | positive | positive | value a/(a + b) |
| Low | c: False | d: True | Negative predictive |
| | negative | negative | value d/(c + d) |
| | Sensitivity | Specificity | |
| | a/(a + c) | d/(b + d) | |

As mentioned previously, a standard value (cut off value) is usually set such that the false positive ratio is low and the sensitivity is high. However, as also apparent from the relationship shown above, there is a trade-off between the false positive ratio and sensitivity. That is, if the standard value (cut off value) is decreased, the detection sensitivity increases. However, since the false positive ratio also increases, it is difficult to satisfy the conditions to have a “low false positive ratio”. Considering this situation, for example, values that give the following predicted results may be selected as the preferable standard values (cut off values) in the present invention.

Standard values (cut off values) for which the false positive ratio is 50% or less (that is, standard values (cut off values) for which the specificity is not less than 50%).

Standard values (cut off values) for which the sensitivity is not less than 20%.

In the present invention, the standard values (cut off values) can be set using a receiver operating characteristic (ROC) curve. An ROC curve is a graph that shows the detection sensitivity on the vertical axis and the false positive ratio (that is, “1-specificity”) on the horizontal axis. In the present invention, an ROC curve can be obtained by plotting the changes in the sensitivity and the false positive ratio, which were obtained after continuously varying the standard value (cut off value) for determining the high/low degree of the blood concentration of Nectin-4.

The “standard value (cut off value)” for obtaining the ROC curve is a value temporarily used for the statistical analyses. The “standard value (cut off value)” for obtaining the ROC curve can generally be continuously varied within a range that is allowed to cover all selectable standard values (cut off value). For example, the standard value (cut off value) can be varied between the smallest and largest measured Nectin-4 values in an analyzed population.

Based on the obtained ROC curve, a preferable standard value (cut off value) to be used in the present invention can be selected from a range that satisfies the above-mentioned conditions. Alternatively, a standard value (cut off value) can be selected based on an ROC curve produced by varying the standard values (cut off values) from a range that includes most of the measured Nectin-4 values. In the present invention, the standard value (cut off value) of the Nectin-4 blood concentration may be set at, for example, 0.6 to 2.0 ng/ml, preferably 0.7 to 1.8 ng/ml, more preferably 0.8 to 1.5 ng/ml, more preferably 0.9 to 1.2 ng/ml, more preferably 1.0 ng/ml.

Nectin-4 in the blood can be measured by any method that can quantitate proteins. For example, immunoassay, liquid chromatography, surface plasmon resonance (SPR), mass spectrometry, or the like can be used in the present invention. In mass spectrometry, proteins can be quantitated by using a suitable internal standard. For example, isotope-labeled Nectin-4 can be used as the internal standard. The concentration of Nectin-4 in the blood can be determined from the peak intensity of Nectin-4 in the blood and that of the internal standard. Generally, the matrix-assisted laser desorption/ionization (MALDI) method is used for mass spectrometry of proteins. With an analysis method that uses mass spectrometry or liquid chromatography, Nectin-4 can also be analyzed simultaneously with other tumor markers (e.g. CEA or CYFRA).

A preferable method for measuring Nectin-4 in the present invention is the immunoassay. The amino acid sequence of Nectin-4 is, for example, shown in SEQ ID NO: 2 (GenBank Accession Number NM₁₃ 030916), and the nucleotide sequence of the cDNA encoding it, for example, is shown in SEQ ID NO: 1. Therefore, those skilled in the art can prepare antibodies by synthesizing necessary immunogens based on the amino acid sequence of Nectin-4. The peptide used as immunogen can be easily synthesized using a peptide synthesizer. The synthetic peptide can be used as an immunogen by linking it to a carrier protein.

Keyhole limpet hemocyanin, myoglobin, albumin, and the like can be used as the carrier protein. Preferable carrier proteins are KLH, bovine serum albumin, and such. The maleimidobenzoyl-N-hydrosuccinimide ester method (hereinafter abbreviated as the MB S method) and the like are generally used to link synthetic peptides to carrier proteins.

Specifically, a cysteine is introduced into the synthetic peptide and the peptide is crosslinked to KLH by MBS using the cysteine's SH group. The cysteine residue may be introduced at the N-terminus or C-terminus of the synthesized peptide.

Alternatively, Nectin-4 can be prepared using the nucleotide sequence of Nectin-4 or a portion thereof. DNAs comprising the necessary nucleotide sequence can be cloned using mRNAs prepared from Nectin-4-expressing tissues. Alternatively, commercially available cDNA libraries can be used as the cloning source. The obtained genetic recombinants of Nectin-4, or fragments thereof, can also be used as the immunogen. Nectin-4 recombinants expressed in this manner are preferable as the immunogen for obtaining the antibodies used in the present invention.

In the present invention, immunologically active fragments originated from the complete Nectin-4 polypeptide, such as epitope peptides, may also be used as immunogens, as well as the complete Nectin-4 polypeptide. The immunologically active fragments to be used as immunogens are not restricted so long as the fragments retain abilities of raising antibodies, and for example, the ectodomain or fragments thereof may be preferably used.

Immunogens obtained in this manner are mixed with a suitable adjuvant and used to immunize animals. Known adjuvants include Freund's complete adjuvant (FCA) and incomplete adjuvant. The immunization procedure is repeated at appropriate intervals until an increase in the antibody titer is confirmed. There are no particular limitations on the immunized animals in the present invention. Specifically, animals commonly used for immunization such as mice, rats, or rabbits can be used.

When obtaining the antibodies as monoclonal antibodies, animals that are advantageous for their production may be used. For example in mice, many myeloma cell lines for cell fusion are known, and techniques for establishing hybridomas with a high probability are already well known. Therefore, mice are a desirable immunized animal to obtain monoclonal antibodies.

Furthermore, the immunization treatments are not limited to in vitro treatments. Methods for immunologically sensitizing cultured immunocompetent cells in vitro can also be employed. Antibody-producing cells obtained by these methods are transformed and cloned. Methods for transforming antibody-producing cells to obtain monoclonal antibodies are not limited to cell fusion. For example, methods for obtaining cloneable transformants by virus infection are known.

Hybridomas that produce the monoclonal antibodies used in the present invention can be screened based on their reactivity to Nectin-4. Specifically, antibody-producing cells are first selected by using as an index the binding activity toward Nectin-4, or a domain peptide thereof, that was used as the immunogen. Positive clones that are selected by this screening are subcloned as necessary.

The monoclonal antibodies to be used in the present invention can be obtained by culturing the established hybridomas under suitable conditions and collecting the produced antibodies. When the hybridomas are homohybridomas, they can be cultured in vivo by inoculating them intraperitoneally in syngeneic animals. In this case, monoclonal antibodies are collected as ascites fluid. When heterohybridomas are used, they can be cultured in vivo using nude mice as a host.

In addition to in vivo cultures, hybridomas are also commonly cultured ex vivo, in a suitable culture environment. For example, basal media such as RPMI 1640 and DMEM are generally used as the medium for hybridomas. Additives such as animal sera can be added to these media to maintain the antibody-producing ability to a high level. When hybridomas are cultured ex vivo, the monoclonal antibodies can be collected as a culture supernatant. Culture supernatants can be collected by separating from cells after culturing, or by continuously collecting while culturing using a culture apparatus that uses a hollow fiber.

Monoclonal antibodies used in the present invention are prepared from monoclonal antibodies collected as ascites fluid or culture supernatants, by separating immunoglobulin fractions by saturated ammonium sulfate precipitation and further purifying by gel filtration, ion exchange chromatography, or such. In addition, if the monoclonal antibodies are IgGs, purification methods based on affinity chromatography with a protein A or protein G column are effective.

On the other hand, to obtain antibodies used in the present invention as polyclonal antibodies, blood is drawn from animals whose antibody titer increased after immunization, and the serum is separated to obtain an anti-serum. Immunoglobulins are purified from anti-sera by known methods to prepare the antibodies used in the present invention. Nectin-4-specific antibodies can be prepared by combining immunoaffinity chromatography which uses Nectin-4 as a ligand with immunoglobulin purification.

When antibodies against Nectin-4 contact Nectin-4, the antibodies bind to the antigenic determinant (epitope) that the antibodies recognize through an antigen-antibody reaction. The binding of antibodies to antigens can be detected by various immunoassay principles. Immunoassays can be broadly categorized into heterogeneous analysis methods and homogeneous analysis methods. To maintain the sensitivity and specificity of immunoassays to a high level, the use of monoclonal antibodies is desirable. Methods of the present invention for measuring Nectin-4 by various immunoassay formats are explained in further detail herein.

First, methods for measuring substance (Nectin-4) using a heterogeneous immunoassay are described. In heterogeneous immunoassays, a mechanism for detecting antibodies that bind to the substance after separating them from those that do not bind to the substance is required.

To facilitate the separation, immobilized reagents are generally used. For example, a solid phase onto which antibodies recognizing the substance have been immobilized is first prepared (immobilized antibodies). The substance is made to bind to these, and secondary antibodies are further reacted thereto.

When the solid phase is separated from the liquid phase and further washed, as necessary, secondary antibodies remain on the solid phase in proportion to the concentration of the substance. By labeling the secondary antibodies, the substance can be quantitated by measuring the signal derived from the label.

Any method may be used to bind the antibodies to the solid phase. For example, antibodies can be physically adsorbed to hydrophobic materials such as polystyrene. Alternatively, antibodies can be chemically bound to a variety of materials having functional groups on their surfaces. Furthermore, antibodies labeled with a binding ligand can be bound to a solid phase by trapping them using a binding partner of the ligand. Combinations of a binding ligand and its binding partner include avidin-biotin and such. The solid phase and antibodies can be conjugated at the same time or before the reaction between the primary antibodies and the substance.

Similarly, the secondary antibodies do not need to be directly labeled. That is, they can be indirectly labeled using antibodies against antibodies or using binding reactions such as that of avidin-biotin.

The concentration of the substance in a sample is determined based on the signal intensities obtained using standard samples with known concentrations of the substance.

Any antibody can be used as the immobilized antibody and secondary antibody for the heterogeneous immunoassays mentioned above, so long as it is an antibody, or a fragment including an antigen-binding site thereof, that recognizes the substance. Therefore, it may be a monoclonal antibody, a polyclonal antibody, or a mixture or combination of both. For example, a combination of monoclonal antibodies and polyclonal antibodies is a preferable combination in the present invention. Alternatively, when both antibodies are monoclonal antibodies, combining monoclonal antibodies recognizing different epitopes is preferable.

Since the antigens to be measured are sandwiched by antibodies, such heterogeneous immunoassays are called sandwich methods. Since sandwich methods excel in the measurement sensitivity and the reproducibility, they are a preferable measurement principle in the present invention.

The principle of competitive inhibition reactions can also be applied to the heterogeneous immunoassays. Specifically, they are immunoassays based on the phenomenon where the substance in a sample competitively inhibits the binding between the substance with a known concentration and an antibody. The concentration of the substance in the sample can be determined by labeling substance with a known concentration and measuring the amount of substance that reacted (or did not react) with the antibody.

A competitive reaction system is established when antigens with a known concentration and antigens in a sample are simultaneously reacted to an antibody. Furthermore, analyses by an inhibitory reaction system are possible when antibodies are reacted with antigens in a sample, and antigens with a known concentration are reacted thereafter. In both types of reaction systems, reaction systems that excel in the operability can be constructed by setting either one of the antigens with a known concentration used as a reagent component or the antibody as the labeled component, and the other one as the immobilized reagent.

Radioisotopes, fluorescent substances, luminescent substances, substances having an enzymatic activity, macroscopically observable substances, magnetically observable substances, and such are used in these heterogeneous immunoassays. Specific examples of these labeling substances are shown below.

Substances having an enzymatic activity:
peroxidase,
alkaline phosphatase,
urease, catalase,
glucose oxidase,
lactate dehydrogenase, or
amylase, etc.
Fluorescent substances:
fluorescein isothiocyanate,
tetramethylrhodamine isothiocyanate,
substituted rhodamine isothiocyanate, or
dichlorotriazine isothiocyanate, etc.
Radioisotopes:
tritium,
¹²⁵I, or
¹³¹I, etc.

Among these, non-radioactive labels such as enzymes are an advantageous label in terms of safety, operability, sensitivity, and such. Enzymatic labels can be linked to antibodies or to Nectin-4 by known methods such as the periodic acid method or maleimide method.

As the solid phase, beads, inner walls of a container, fine particles, porous carriers, magnetic particles, or such are used. Solid phases formed using materials such as polystyrene, polycarbonate, polyvinyl toluene, polypropylene, polyethylene, polyvinyl chloride, nylon, polymethacrylate, latex, gelatin, agarose, glass, metal, ceramic, or such can be used. Solid materials in which functional groups to chemically bind antibodies and such have been introduced onto the surface of the above solid materials are also known. Known binding methods, including chemical binding such as poly-L-lysine or glutaraldehyde treatment and physical adsorption, can be applied for solid phases and antibodies (or antigens).

Although the steps of separating the solid phase from the liquid phase and the washing steps are required in all heterogeneous immunoassays exemplified herein, these steps can easily be performed using the immunochromatography method, which is a variation of the sandwich method.

Specifically, antibodies to be immobilized are immobilized onto porous carriers capable of transporting a sample solution by the capillary phenomenon, then a mixture of a sample comprising substance (Nectin-4) and labeled antibodies is deployed therein by this capillary phenomenon. During deployment, substance reacts with the labeled antibodies, and when it further contacts the immobilized antibodies, it is trapped at that location. The labeled antibodies that do not react with the substance pass through, without being trapped by the immobilized antibodies.

As a result, the presence of the substance can be detected using, as an index, the signals of the labeled antibodies that remain at the location of the immobilized antibodies. If the labeled antibodies are maintained upstream in the porous carrier in advance, all reactions can be initiated and completed by just dripping in the sample solutions, and an extremely simple reaction system can be constructed. In the immunochromatography method, labeled components that can be distinguished macroscopically, such as colored particles, can be combined to construct an analytical device that does not even require a special reader.

Furthermore, in the immunochromatography method, the detection sensitivity for the substance can be adjusted. For example, by adjusting the detection sensitivity near the cutoff value described below, the aforementioned labeled components can be detected when the cutoff value is exceeded. By using such a device, whether a subject is positive or negative can be judged very simply. By adopting a constitution that allows a macroscopic distinction of the labels, necessary examination results can be obtained by simply applying blood samples to the device for immunochromatography.

Various methods for adjusting the detection sensitivity of the immunochromatography method are known in the art. For example, a second immobilized antibody for adjusting the detection sensitivity can be placed between the position where samples are applied and the immobilized antibodies (Japanese Patent Application Kokai Publication No. (JP-A) H06-341989 (unexamined, published Japanese patent application)). The substance in the sample is trapped by the second immobilized antibody while deploying from the position where the sample was applied to the position of the first immobilized antibody for label detection. After the second immobilized antibody is saturated, the substance can reach the position of the first immobilized antibody located downstream. As a result, when the concentration of the substance comprised in the sample exceeds a predetermined concentration, the substance bound to the labeled antibody is detected at the position of the first immobilized antibody.

Next, homogeneous immunoassays are described. As opposed to heterogeneous immunological assay methods that require a separation of the reaction solutions as described above, substance (Nectin-4) can also be measured using homogeneous analysis methods. Homogeneous analysis methods allow the detection of antigen-antibody reaction products without their separation from the reaction solutions.

A representative homogeneous analysis method is the immunoprecipitation reaction, in which antigenic substances are quantitatively analyzed by examining precipitates produced following an antigen-antibody reaction. Polyclonal antibodies are generally used for the immunoprecipitation reactions. When monoclonal antibodies are applied, multiple types of monoclonal antibodies that bind to different epitopes of the substance are preferably used. The products of precipitation reactions that follow the immunological reactions can be macroscopically observed or can be optically measured for conversion into numerical data.

The immunological particle agglutination reaction, which uses as an index the agglutination by antigens of antibody-sensitized fine particles, is a common homogeneous analysis method. As in the aforementioned immunoprecipitation reaction, polyclonal antibodies or a combination of multiple types of monoclonal antibodies can be used in this method as well. Fine particles can be sensitized with antibodies through sensitization with a mixture of antibodies, or they can be prepared by mixing particles sensitized separately with each antibody. Fine particles obtained in this manner gives matrix-like reaction products upon contact with the substance. The reaction products can be detected as particle aggregation. Particle aggregation may be macroscopically observed or can be optically measured for conversion into numerical data.

Immunological analysis methods based on energy transfer and enzyme channeling are known as homogeneous immunoassays. In methods utilizing energy transfer, different optical labels having a donor/acceptor relationship are linked to multiple antibodies that recognize adjacent epitopes on an antigen. When an immunological reaction takes place, the two parts approach and an energy transfer phenomenon occurs, resulting in a signal such as quenching or a change in the fluorescence wavelength. On the other hand, enzyme channeling utilizes labels for multiple antibodies that bind to adjacent epitopes, in which the labels are a combination of enzymes having a relationship such that the reaction product of one enzyme is the substrate of another. When the two parts approach due to an immunological reaction, the enzyme reactions are promoted; therefore, their binding can be detected as a change in the enzyme reaction rate.

In the present invention, blood for measuring Nectin-4 can be prepared from blood drawn from patients. Preferable blood samples are the serum or plasma. Serum or plasma samples can be diluted before the measurements. Alternatively, the whole blood can be measured as a sample and the obtained measured value can be corrected to determine the serum concentration. For example, concentration in whole blood can be corrected to the serum concentration by determining the percentage of corpuscular volume in the same blood sample.

In a preferred embodiment, the immunoassay comprises an ELISA. The present inventors established sandwich ELISA to detect serum Nectin-4 in patients with lung cancer.

The Nectin-4 level in the blood samples is then compared with a Nectin-4 level associated with a reference sample such as a normal control sample. The phrase “normal control level” refers to the level of Nectin-4 typically found in a blood sample of a population not suffering from lung cancer, respectively. The reference sample is preferably of a similar nature to that of the test sample. For example, if the test samples include patient serum, the reference sample should also be serum. The Nectin-4 level in the blood samples from control and test subjects may be determined at the same time or, alternatively, the normal control level may be determined by a statistical method based on the results obtained by analyzing the level of Nectin-4 in samples previously collected from a control group.

The Nectin-4 level may also be used to monitor the course of treatment of lung cancer. In this method, a test blood sample is provided from a subject undergoing treatment for lung cancer. Preferably, multiple test blood samples are obtained from the subject at various time points, including before, during, and/or after the treatment. The level of Nectin-4 in the post-treatment sample may then be compared with the level of Nectin-4 in the pre-treatment sample or, alternatively, with a reference sample (e.g., a normal control level). For example, if the post-treatment Nectin-4 level is lower than the pre-treatment Nectin-4 level, one can conclude that the treatment was efficacious. Likewise, if the post-treatment Nectin-4 level is similar to the normal control Nectin-4 level, one can also conclude that the treatment was efficacious.

An “efficacious” treatment is one that leads to a reduction in the level of Nectin-4 or a decrease in size, prevalence, or metastatic potential of lung cancer in a subject. When a treatment is applied prophylactically, “efficacious” means that the treatment retards or prevents occurrence of lung cancer or alleviates a clinical symptom of lung cancer. The assessment of lung cancer can be made using standard clinical protocols. Furthermore, the efficaciousness of a treatment can be determined in association with any known method for diagnosing or treating lung cancer. For example, lung cancer is routinely diagnosed histopathologically or by identifying symptomatic anomalies.

Kit for the Serological Diagnosis of Lung Cancer:

Components used to carry out the diagnosis of cancer according to the present invention can be combined in advance and supplied as a testing kit. Accordingly, the present invention provides a kit for detecting cancer, which relates to Nectin-4 overexpression, including:

(i) an immunoassay reagent for determining a level of Nectin-4 in a blood sample.

In the preferable embodiments, the kit of the present invention may further comprise:

(ii) a positive control sample for Nectin-4.

In the preferable embodiments, the kit of the present invention may further comprise:

(iii) an immunoassay reagent for determining a level of CEA or CYFRA in a blood sample.

In the preferable embodiments, the kit of the present invention may further comprise:

(iv) a positive control sample for CEA and/or CYFRA.

The kit of the present invention may be preferably applicable to lung cancer, bladder cancer and cervical carcinoma, more preferably lung cancer, further more preferably NSCLCs.

The reagents for the immunoassays which constitute a kit of the present invention may include reagents necessary for the various immunoassays described above. Specifically, the reagents for the immunoassays include an antibody that recognizes the substance to be measured. The antibody can be modified depending on the assay format of the immunoassay. ELISA can be used as a preferable assay format of the present invention. In ELISA, for example, a first antibody immobilized onto a solid phase and a second antibody having a label are generally used.

Therefore, the immunoassay reagents for ELISA can include a first antibody immobilized onto a solid phase carrier. Fine particles or the inner walls of a reaction container can be used as the solid phase carrier. Magnetic particles can be used as the fine particles. Alternatively, multi-well plates such as 96-well microplates are often used as the reaction containers. Containers for processing a large number of samples, which are equipped with wells having a smaller volume than in 96-well microplates at a high density, are also known. In the present invention, the inner walls of these reaction containers can be used as the solid phase carriers.

The immunoassay reagents for ELISA may further include a second antibody having a label. The second antibody for ELISA may be an antibody onto which an enzyme is directly or indirectly linked. Methods for chemically linking an enzyme to an antibody are known. For example, immunoglobulins can be enzymatically cleaved to obtain fragments comprising the variable regions. By reducing the —SS— bonds comprised in these fragments to —SH groups, bifunctional linkers can be attached. By linking an enzyme to the bifunctional linkers in advance, enzymes can be linked to the antibody fragments.

Alternatively, to indirectly link an enzyme, for example, the avidin-biotin binding can be used. That is, an enzyme can be indirectly linked to an antibody by contacting a biotinylated antibody with an enzyme to which avidin has been attached. In addition, an enzyme can be indirectly linked to a second antibody using a third antibody which is an enzyme-labeled antibody recognizing the second antibody. For example, enzymes such as those exemplified above can be used as the enzymes to label the antibodies.

Kits of the present invention include a positive control for Nectin-4. A positive control for Nectin-4 includes Nectin-4 whose concentration has been determined in advance. Preferable concentrations are, for example, a concentration set as the standard value (e.g., 1.0 ng/ml as the cut off value) in a testing method of the present invention. Alternatively, a positive control having a higher concentration can also be combined. The positive control for Nectin-4 in the present invention can additionally comprise CEA and/or CYFRA whose concentration has been determined in advance. A positive control comprising Nectin-4, CEA and/or CYFRA is preferable as the positive control of the present invention.

Therefore, the present invention provides a positive control for detecting cancer, which includes Nectin-4 and CEA and/or CYFRA at concentrations above a normal value. Alternatively, the present invention relates to the use of a blood sample including Nectin-4 and CEA and/or CYFRA at concentrations above a normal value in the production of a positive control for the detection of cancer. It has been known that CEA and/or CYFRA can serve as an index for cancer; however, that Nectin-4 can serve as an index for lung cancer is a novel finding obtained by the present invention. Therefore, positive controls including Nectin-4 in addition to CEA and/or CYFRA are novel. The positive controls of the present invention can be prepared by adding CEA and/or CYFRA and Nectin-4 at concentrations above a standard value to blood samples. For example, sera comprising CEA and/or CYFRA and Nectin-4 at concentrations above a standard value are preferable as the positive controls of the present invention.

The positive controls in the present invention are preferably in a liquid form. In the present invention, blood samples are used as samples. Therefore, samples used as controls also need to be in a liquid form. Alternatively, by dissolving a dried positive control with a predefined amount of liquid at the time of use, a control that gives the tested concentration can be prepared. By packaging, together with a dried positive control, an amount of liquid necessary to dissolve it, the user can obtain the necessary positive control by just mixing them. Nectin-4 used as the positive control can be a naturally-derived protein or it may be a recombinant protein. Not only positive controls, but also negative controls can be combined in the kits of the present invention. The positive controls or negative controls are used to verify that the results indicated by the immunoassays are correct.

Method for Assessing the Prognosis of Cancer:

The present invention relates to the novel discovery that Nectin-4 expression is significantly associated with poorer prognosis of cancer patients. Thus, the present invention provides a method for determining or assessing the prognosis of a patient with cancer, in particular lung cancer, by detecting the expression level of the Nectin-4 gene in a biological sample of the patient; comparing the detected expression level to a control level; and determining a increased expression level to the control level as indicative of poor prognosis (poor survival).

Herein, the term “prognosis” refers to a forecast as to the probable outcome of the disease as well as the prospect of recovery from the disease as indicated by the nature and symptoms of the case. Accordingly, a less favorable, negative, poor prognosis is defined by a lower post-treatment survival term or survival rate. Conversely, a positive, favorable, or good prognosis is defined by an elevated post-treatment survival term or survival rate.

The terms “assessing the prognosis” refer to the ability of predicting, forecasting or correlating a given detection or measurement with a future outcome of cancer of the patient (e.g., malignancy, likelihood of curing cancer, survival, and the like). For example, a determination of the expression level of Nectin-4 over time enables a predicting of an outcome for the patient (e.g., increase or decrease in malignancy, increase or decrease in grade of a cancer, likelihood of curing cancer, survival, and the like).

In the context of the present invention, the phrase “assessing (or determining) the prognosis” is intended to encompass predictions and likelihood analysis of cancer, progression, particularly cancer recurrence, metastatic spread and disease relapse. The present method for assessing prognosis is intended to be used clinically in making decisions concerning treatment modalities, including therapeutic intervention, diagnostic criteria such as disease staging, and disease monitoring and surveillance for metastasis or recurrence of neoplastic disease.

The patient-derived biological sample used for the method may be any sample derived from the subject to be assessed so long as the Nectin-4 gene can be detected in the sample. Preferably, the biological sample is a lung cell (a cell obtained from the lung). Furthermore, the biological sample may include biopsy specimen, bodily fluids such as sputum, blood, serum, or plasma. Moreover, the sample may be cells purified from a tissue. The biological samples may be obtained from a patient at various time points, including before, during, and/or after a treatment.

According to the present invention, it was shown that the higher the expression level of the Nectin-4 gene measured in the patient-derived biological sample, the poorer the prognosis for post-treatment remission, recovery, and/or survival and the higher the likelihood of poor clinical outcome. Thus, according to the present method, the “control level” used for comparison may be, for example, the expression level of the Nectin-4 gene detected before any kind of treatment in an individual or a population of individuals who showed good or positive prognosis of cancer, after the treatment, which herein will be referred to as “good prognosis control level”. Alternatively, the “control level” may be the expression level of the Nectin-4 gene detected before any kind of treatment in an individual or a population of individuals who showed poor or negative prognosis of cancer, after the treatment, which herein will be referred to as “poor prognosis control level”. The “control level” is a single expression pattern derived from a single reference population or from a plurality of expression patterns. Thus, the control level may be determined based on the expression level of the Nectin-4 gene detected before any kind of treatment in a patient of cancer, or a population of the patients whose disease state (good or poor prognosis) is known. Preferably, cancer is lung cancer, bladder cancer and cervical carcinoma, more preferably lung cancer, further more preferably NSCLCs. It is preferred, to use the standard value of the expression levels of the Nectin-4 gene in a patient group with a known disease state. The standard value may be obtained by any method known in the art. For example, a range of mean+/−2 S.D. or mean+/−3 S.D. may be used as standard value. Alternatively, the standard values can be obtained based on ROC curves, and the standard values obtained by this manner, are usually referred to as “cut off value”. In cases where the expression level of Nectin-4 gene is detected as a level of Nectin-4 in a blood sample, the cut off value may be set at, for example, 0.6 to 2.0 ng/ml, preferably 0.7 to 1.8 ng/ml, more preferably 0.8 to 1.5 ng/ml, further more preferably 0.9 to 1.2 ng/ml, further more preferably 1.0 ng/ml.

The control level may be determined at the same time with the test biological sample by using a sample(s) previously collected and stored before any kind of treatment from cancer patient(s) (control or control group) whose disease state (good prognosis or poor prognosis) are known.

Alternatively, the control level may be determined by a statistical method based on the results obtained by analyzing the expression level of the Nectin-4 gene in samples previously collected and stored from a control group. Furthermore, the control level can be a database of expression patterns from previously tested cells.

Moreover, according to an aspect of the present invention, the expression level of the Nectin-4 gene in a biological sample may be compared to multiple control levels, which control levels are determined from multiple reference samples. It is preferred to use a control level determined from a reference sample derived from a tissue type similar to that of the patient-derived biological sample.

According to the present invention, a similarity in the expression level of the Nectin-4 gene to a good prognosis control level indicates a more favorable prognosis of the patient and an increase in the expression level to the good prognosis control level indicates less favorable, poorer prognosis for post-treatment remission, recovery, survival, and/or clinical outcome. On the other hand, a decrease in the expression level of the Nectin-4 gene to the poor prognosis control level indicates a more favorable prognosis of the patient and a similarity in the expression level to the poor prognosis control level indicates less favorable, poorer prognosis for post-treatment remission, recovery, survival, and/or clinical outcome.

The expression level of the Nectin-4 gene in a biological sample can be considered altered when the expression level differs from the control level by more than 1.0, 1.5, 2.0, 5.0, 10.0, or more fold.

The difference in the expression level between the test biological sample and the control level can be normalized to a control, e.g., housekeeping gene. For example, polynucleotides whose expression levels are known not to differ between the cancerous and non-cancerous cells, including those coding for beta-actin, glycer-aldehyde 3-phosphate dehydrogenase, and ribosomal protein P1, may be used to normalize the expression levels of the Nectin-4 genes.

The expression level may be determined by detecting the gene transcript in the patient-derived biological sample using techniques well known in the art. The gene transcripts detected by the present method include both the transcription and translation products, such as mRNA and protein.

For instance, the transcription product of the Nectin-4 gene can be detected by hybridization, e.g., Northern blot hybridization analyses, that use a Nectin-4 gene probe to the gene transcript. The detection may be carried out on a chip or an array. The use of an array is preferable for detecting the expression level of a plurality of genes including the Nectin-4 gene. As another example, amplification-based detection methods, such as reverse-transcription based polymerase chain reaction (RT-PCR) which use primers specific to the Nectin-4 gene may be employed for the detection (see Example). The Nectin-4 gene-specific probe or primers may be designed and prepared using conventional techniques by referring to the whole sequence of the Nectin-4 gene (SEQ ID NO: 1). For example, the primers (SEQ ID NOs: 3 and 4) used in the Example may be employed for the detection by RT-PCR, but the present invention is not restricted thereto.

Specifically, a probe or primer used for the present method hybridizes under stringent, moderately stringent, or low stringent conditions to the mRNA of the Nectin-4 gene. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degree Centigrade lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degrees C. for short probes or primers (e.g., 10 to 50 nucleotides) and at least about 60 degrees C. for longer probes or primers. Stringent conditions may also be achieved with the addition of destabilizing agents, such as formamide.

Alternatively, the translation product may be detected for the assessment of the present invention. For example, the quantity of the Nectin-4 protein may be determined. A method for determining the quantity of the protein as the translation product includes immunoassay methods that use an antibody specifically recognizing the Nectin-4 protein. The antibody may be monoclonal or polyclonal. Furthermore, any fragment or modification (e.g., chimeric antibody, scFv, Fab, F(ab′)2, Fv, etc.) of the antibody may be used for the detection, so long as the fragment retains the binding ability to the Nectin-4 protein. Methods to prepare these kinds of antibodies for the detection of proteins are well known in the art, and any method may be employed in the present invention to prepare such antibodies and equivalents thereof.

As another method to detect the expression level of the Nectin-4 gene based on its translation product, the intensity of staining may be observed via immunohistochemical analysis using an antibody against Nectin-4 protein. Namely, the observation of strong staining indicates increased presence of the Nectin-4 protein and at the same time high expression level of the Nectin-4 gene.

Alternatively, the expression level of the Nectin-4 gene may be detected as a level of Nectin-4 in a blood sample derived from a patient, as the level of Nectin-4 in a blood sample (e.g., serum) significantly correlates with prognosis. The level of Nectin-4 in a blood sample may be measured by the methods described above in the item of Serological diagnosis of cancer.

Furthermore, the Nectin-4 protein is known to have a cell proliferating activity. Therefore, the expression level of the Nectin-4 gene can be determined using such cell proliferating activity as an index. For example, cells which express Nectin-4 are prepared and cultured in the presence of a biological sample, and then by detecting the speed of proliferation, or by measuring the cell cycle or the colony forming ability the cell proliferating activity of the biological sample can be determined.

Alternatively, according to the present invention, an intermediate result may also be provided in addition to other test results for assessing the prognosis of a subject. Such intermediate result may assist a doctor, nurse, or other practitioner to assess, determine, or estimate the prognosis of a subject. Additional information that may be considered, in combination with the intermediate result obtained by the present invention, to assess prognosis includes clinical symptoms and physical conditions of a subject.

The patient to be assessed for the prognosis of cancer according to the method is preferably a mammal and includes human, non-human primate, mouse, rat, dog, cat, horse, and cow.

A Kit for Diagnosing Cancer or Assessing the Prognosis of Cancer:

The present invention provides a kit for diagnosing cancer or assessing the prognosis of cancer. Preferably, the cancer is lung cancer, bladder cancer and cervical carcinoma, more preferably lung cancer, further more preferably NSCLCs. Specifically, the kit includes at least one reagent for detecting the expression of the Nectin-4 gene in a patient-derived biological sample, which reagent may be selected from the group of:

(a) a reagent for detecting mRNA of the Nectin-4 gene;

(b) a reagent for detecting the Nectin-4 protein; and

(c) a reagent for detecting the biological activity of the Nectin-4 protein.

Suitable reagents for detecting mRNA of the Nectin-4 gene include nucleic acids that specifically bind to or identify the Nectin-4 mRNA, such as oligonucleotides which have a complementary sequence to a part of the Nectin-4 mRNA. These kinds of oligonucleotides are exemplified by primers and probes that are specific to the Nectin-4 mRNA. These kinds of oligonucleotides may be prepared based on methods well known in the art. If needed, the reagent for detecting the Nectin-4 mRNA may be immobilized on a solid matrix. Moreover, more than one reagent for detecting the Nectin-4 mRNA may be included in the kit.

On the other hand, suitable reagents for detecting the Nectin-4 protein include antibodies to the Nectin-4 protein. The antibody may be monoclonal or polyclonal. Furthermore, any fragment or modification (e.g., chimeric antibody, scFv, Fab, F(ab′)2, Fv, etc.) of the antibody may be used as the reagent, so long as the fragment retains the binding ability to the Nectin-4 protein. Methods to prepare these kinds of antibodies for the detection of proteins are well known in the art, and any method may be employed in the present invention to prepare such antibodies and equivalents thereof. Furthermore, the antibody may be labeled with signal generating molecules via direct linkage or an indirect labeling technique. Labels and methods for labeling antibodies and detecting the binding of antibodies to their targets are well known in the art and any labels and methods may be employed for the present invention. Moreover, more than one reagent for detecting the Nectin-4 protein may be included in the kit.

Furthermore, the biological activity can be determined by, for example, measuring the cell proliferating activity due to the expressed Nectin-4 protein in the biological sample. For example, the cell is cultured in the presence of a patient-derived biological sample, and then by detecting the speed of proliferation, or by measuring the cell cycle or the colony forming ability the cell proliferating activity of the biological sample can be determined. If needed, the reagent for detecting the Nectin-4 mRNA may be immobilized on a solid matrix. Moreover, more than one reagent for detecting the biological activity of the Nectin-4 protein may be included in the kit.

The kit may contain more than one of the aforementioned reagents. Furthermore, the kit may include a solid matrix and reagent for binding a probe against the Nectin-4 gene or antibody against the Nectin-4 protein, a medium and container for culturing cells, positive and negative control reagents, and a secondary antibody for detecting an antibody against the Nectin-4 protein. For example, tissue samples obtained from patient with good prognosis or poor prognosis may serve as useful control reagents. A kit of the present invention may further include other materials desirable from a commercial and user standpoint, including buffers, diluents, filters, needles, syringes, and package inserts (e.g., written, tape, CD-ROM, etc.) with instructions for use. These reagents and such may be comprised in a container with a label. Suitable containers include bottles, vials, and test tubes. The containers may be formed from a variety of materials, such as glass or plastic.

As an embodiment of the present invention, when the reagent is a probe against the Nectin-4 mRNA, the reagent may be immobilized on a solid matrix, such as a porous strip, to form at least one detection site. The measurement or detection region of the porous strip may include a plurality of sites, each containing a nucleic acid (probe). A test strip may also contain sites for negative and/or positive controls. Alternatively, control sites may be located on a strip separated from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of Nectin-4 mRNA present in the sample. The detection sites may be configured in any suitably detectable shape and are typically in the shape of a bar or dot spanning the width of a test strip.

The kit of the present invention may further include a positive control sample or Nectin-4 standard sample. The positive control sample of the present invention may be prepared by collecting Nectin-4 positive samples and then those Nectin-4 level are assayed. Alternatively, purified Nectin-4 protein or polynucleotide may be transfected to cell to form the positive sample or the Nectin-4 standard.

In another embodiment, the kit of the present invention may further include a negative control sample. The negative control sample of the present invention is non-Nectin-4 expressing cells or tissue.

In another embodiment, the kit, in the case for assessing or determining the prognosis, may further include a good prognosis control sample and/or a poor prognosis control sample. The good prognosis control sample may be prepared from biological samples derived from patients before any kind of treatment, wherein the patients is known to have showed good or positive prognosis after the treatment. On the other hand, the poor prognosis control sample may be prepared from biological samples derived from patients before any kind of treatment wherein the patients is known to have showed poor or negative prognosis after treatment. The biological samples to be prepared control samples are not limited to, and preferably lung tissue samples or blood samples such as serum. Alternatively, samples which contain the standard value of the transcription or translation product of the Nectin-4 gene may preferably be used as control samples. The standard value may be obtained by any method known in the art. For example, a range of mean+/−2 S.D. or mean+/−3 S.D. may be used as standard value. Alternatively, the standard values can be obtained based on ROC curves, and the standard values obtained by this manner, are usually referred to as “cut off value”. In cases where the expression level of Nectin-4 gene is detected as a level of Nectin-4 in a blood sample, the cut off value may be set at, for example, 0.6 to 2.0 ng/ml, preferably 0.7 to 1.8 ng/ml, more preferably 0.8 to 1.5 ng/ml, further more preferably 0.9 to 1.2 ng/ml, further more preferably 1.0 ng/ml.

In preferred embodiment, a level of Nectin-4 in a blood sample may be detected as the index of the expression level of the Nectin-4 gene. Therefore, the kit of the present invention preferably includes immunoassay regents for detecting a level of Nectin-4 in a blood sample. The preferable reagents for the immunoassays are described above in the item “Kit for the serological diagnosis of lung cancer”

Double-Stranded Molecules:

As used herein, the term “isolated double-stranded molecule” refers to a nucleic acid molecule that inhibits expression of a target gene and includes, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)).

As used herein, the term “siRNA” refers to a double-stranded RNA molecule which prevents translation of a target mRNA. Standard techniques of introducing siRNA into the cell are used, including those in which DNA is a template from which RNA is transcribed. The siRNA includes a Nectin-4 sense nucleic acid sequence (also referred to as “sense strand”), a Nectin-4 antisense nucleic acid sequence (also referred to as “antisense strand”) or both. The siRNA may be constructed such that a single transcript has both the sense and complementary antisense nucleic acid sequences of the target gene, e.g., a hairpin. The siRNA may either be a dsRNA or shRNA.

As used herein, the term “dsRNA” refers to a construct of two RNA molecules composed of complementary sequences to one another and that have annealed together via the complementary sequences to form a double-stranded RNA molecule. The nucleotide sequence of two strands may include not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, composed of first and second regions complementary to one another, i.e., sense and antisense strands. The degree of complementarity and orientation of the regions are sufficient such that base pairing occurs between the regions, the first and second regions are joined by a loop region, the loop results from a lack of base pairing between nucleotides (or nucleotide analogs) within the loop region. The loop region of an shRNA is a single-stranded region intervening between the sense and antisense strands and may also be referred to as “intervening single-strand”.

As use herein, the term “siD/R-NA” refers to a double-stranded polynucleotide molecule which is composed of both RNA and DNA, and includes hybrids and chimeras of RNA and DNA and prevents translation of a target mRNA. Herein, a hybrid indicates a molecule wherein a polynucleotide composed of DNA and a polynucleotide composed of RNA hybridize to each other to form the double-stranded molecule; whereas a chimera indicates that one or both of the strands composing the double stranded molecule may contain RNA and DNA. Standard techniques of introducing siD/R-NA into the cell are used. The siD/R-NA includes a Nectin-4 sense nucleic acid sequence (also referred to as “sense strand”), a Nectin-4 antisense nucleic acid sequence (also referred to as “antisense strand”) or both. The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules composed of complementary sequences to one another and that have annealed together via the complementary sequences to form a double-stranded polynucleotide molecule. The nucleotide sequence of two strands may comprise not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, composed of the first and second regions complementary to one another, i.e., sense and antisense strands. The degree of complementarity and orientation of the regions are sufficient such that base pairing occurs between the regions, the first and second regions are joined by a loop region, and the loop results from a lack of base pairing between nucleotides (or nucleotide analogs) within the loop region. The loop region of an shD/R-NA is a single-stranded region intervening between the sense and antisense strands and may also be referred to as “intervening single-strand”.

As used herein, an “isolated nucleic acid” is a nucleic acid removed from its original environment (e.g., the natural environment if naturally occurring) and thus, synthetically altered from its natural state. In the present invention, examples of isolated nucleic acid include DNA, RNA, and derivatives thereof.

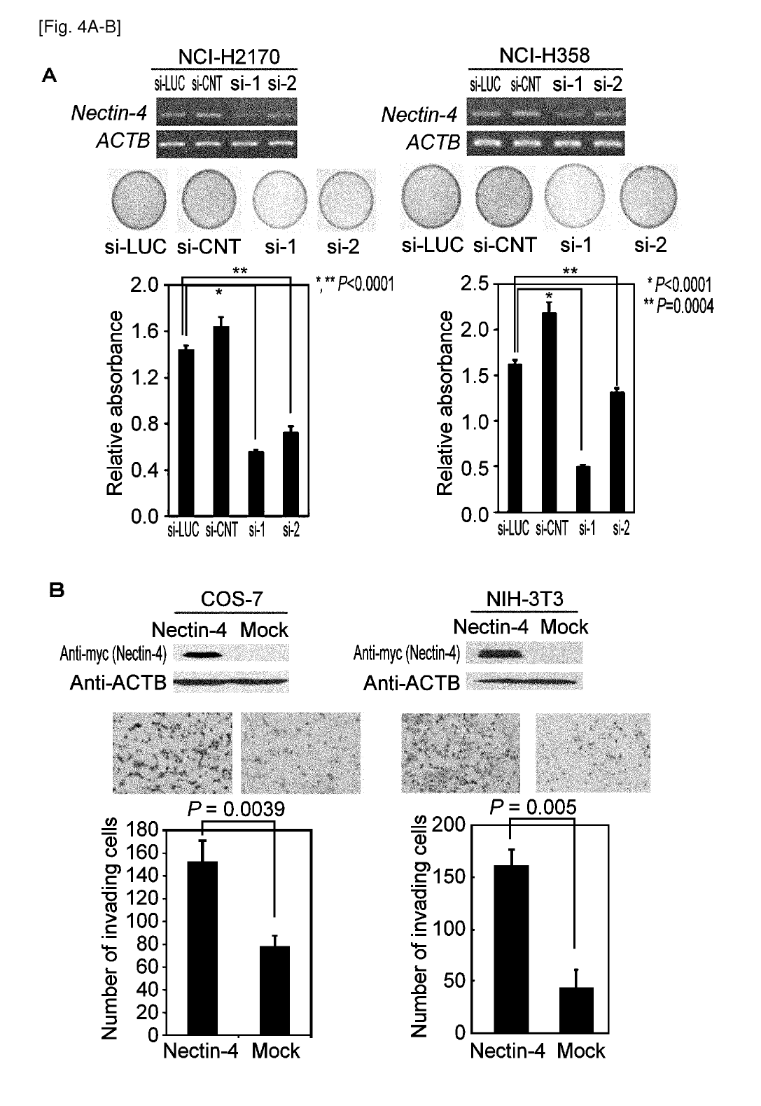

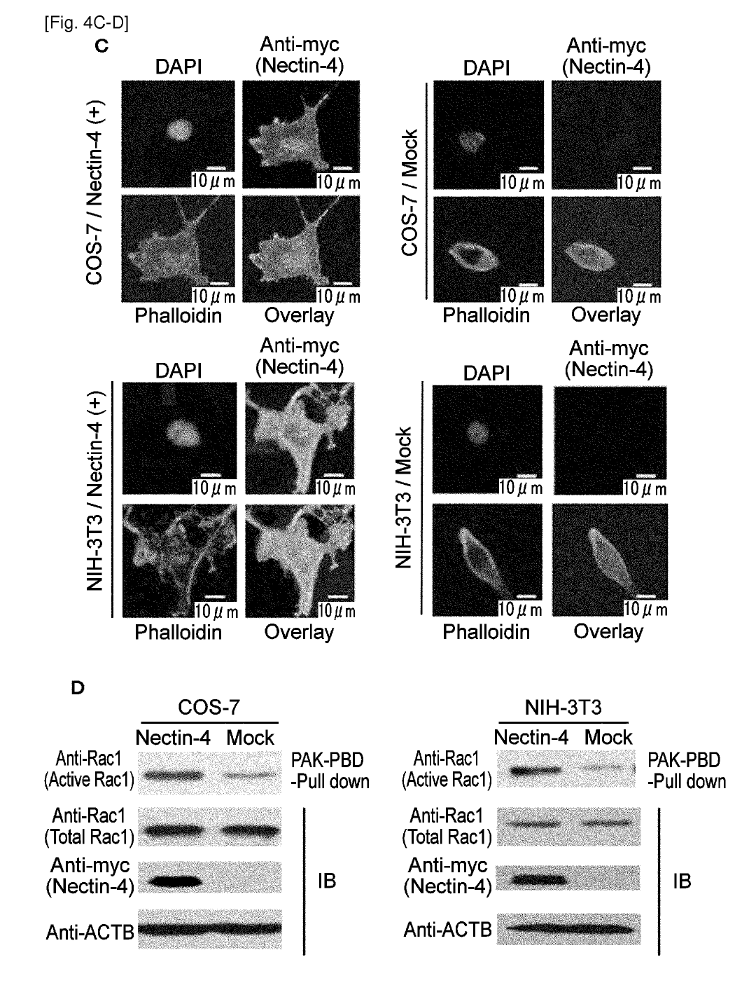

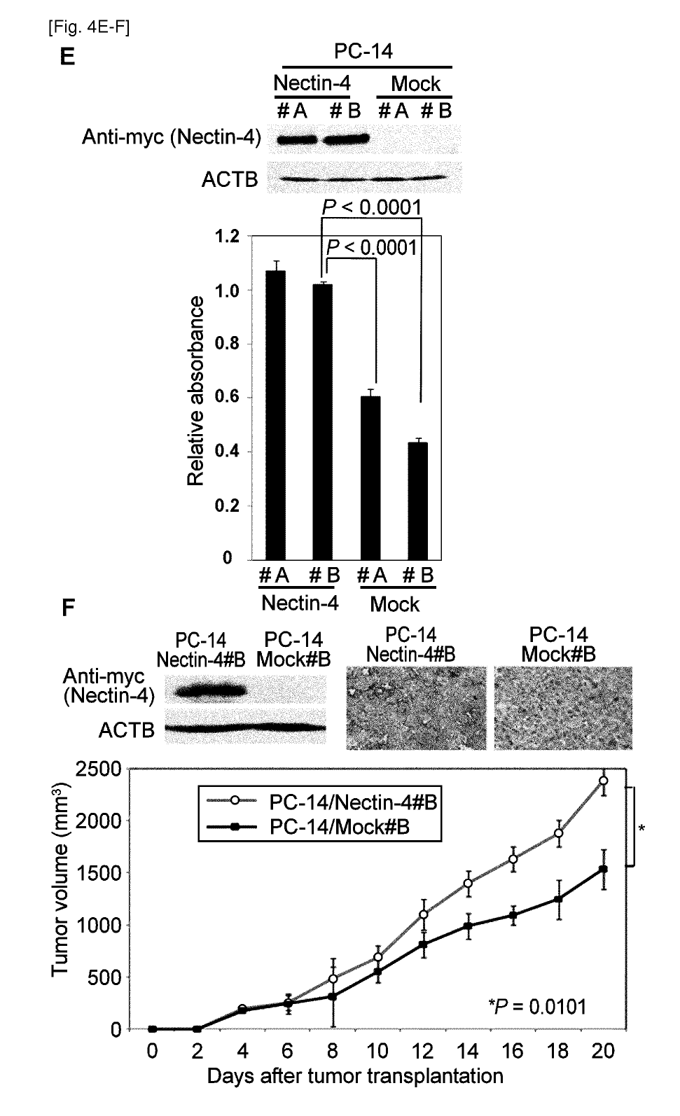

A double-stranded molecule against Nectin-4, which molecule hybridizes to target mRNA, decreases or inhibits production of Nectin-4 protein encoded by Nectin-4 gene by associating with the normally single-stranded mRNA transcript of the gene, thereby interfering with translation and thus, inhibiting expression of the protein. As demonstrated herein, the expression of Nectin-4 in lung cancer cell lines was inhibited by dsRNA (FIG. 4A).

Therefore the present invention provides isolated double-stranded molecules that are capable of inhibiting the inhibit expression of Nectin-4 gene when introduced into a cell expressing the gene. The target sequence of double-stranded molecule may be designed by an siRNA design algorithm such as that mentioned below.

Nectin-4 target sequence includes, for example, nucleotide sequence of SEQ ID NO: 10, or SEQ ID NO: 11.

Specifically, the present invention includes the following double-stranded molecules [1] to [19]:

[1] An isolated double-stranded molecule that, when introduced into a cell, inhibits expression of Nectin-4 and cell proliferation, such molecules composed of a sense strand and an antisense strand complementary thereto, hybridized to each other to form the double-stranded molecule, wherein the sense strand comprises the oligonucleotide corresponding to SEQ ID NO: 1 or fragment thereof;

[2] The double-stranded molecule of [1], wherein the sense strand comprises a sequence corresponding to a target sequence selected from among SEQ ID NOs: 10 and 11;

[3] The double-stranded molecule of [2], having a length of less than about 100 nucleotides;

[4] The double-stranded molecule of [3], having a length of less than about 75 nucleotides;

[5] The double-stranded molecule of [4], having a length of less than about 50 nucleotides;

[6] The double-stranded molecule of [5] having a length of less than about 25 nucleotides;

[7] The double-stranded molecule of [6], having a length of between about 19 and about 25 nucleotides;

[8] The double-stranded molecule of [1], composed of a single polynucleotide having both the sense and antisense strands linked by an intervening single-strand;

[9] The double-stranded molecule of [8], having the general formula 5′-[A]-[B]-[A′]-3′, wherein [A] is the sense strand containing a sequence corresponding to a target sequence selected from among SEQ ID NOs: 10 and 11, [B] is the intervening single-strand composed of 3 to 23 nucleotides, and [A′] is the antisense strand containing a sequence complementary to [A];

[10] The double-stranded molecule of [1], composed of RNA;

[11] The double-stranded molecule of [1], composed of both DNA and RNA;

[12] The double-stranded molecule of [11], wherein the molecule is a hybrid of a DNA polynucleotide and an RNA polynucleotide;

[13] The double-stranded molecule of [12] wherein the sense and the antisense strands are composed of DNA and RNA, respectively;

[14] The double-stranded molecule of [11], wherein the molecule is a chimera of DNA and RNA;

[15] The double-stranded molecule of [14], wherein a region flanking to the 3′-end of the antisense strand, or both of a region flanking to the 5′-end of sense strand and a region flanking to the 3′-end of antisense strand are RNA;

[16] The double-stranded molecule of [15], wherein the flanking region is composed of 9 to 13 nucleotides; and

[17] The double-stranded molecule of [1], wherein the molecule contains 3′ overhang;

[18] A vector expressing the double-stranded molecule of [1];

[19] The vector of [18], wherein the double-stranded molecule has the general formula 5′-[A]-[B]-[A′]-3′, wherein [A] is the sense strand contains a sequence corresponding to a target sequence selected from among SEQ ID NOs: 10 and 11, [B] is an intervening single-strand is composed of 3 to 23 nucleotides, and [A′] is the antisense strand contains a sequence complementary to [A].

The double-stranded molecule of the present invention will be described in more detail below.

Methods for designing double-stranded molecules having the ability to inhibit target gene expression in cells are known. (See, for example, U.S. Pat. No. 6,506,559, herein incorporated by reference in its entirety). For example, a computer program for designing siRNAs is available from the Ambion website (http://www.ambion.com/techlib/misc/siRNA_finder.html).

The computer program selects target nucleotide sequences for double-stranded molecules based on the following protocol.

Selection of Target Sites:

1. Beginning with the AUG start codon of the transcript, scan downstream for AA dinucleotide sequences. Record the occurrence of each AA and the 3′ adjacent 19 nucleotides as potential siRNA target sites. Tuschl et al. recommend to avoid designing siRNA to the 5′ and 3′ untranslated regions (UTRs) and regions near the start codon (within 75 bases) as these may be richer in regulatory protein binding sites, and UTR-binding proteins and/or translation initiation complexes may interfere with binding of the siRNA endonuclease complex.

2. Compare the potential target sites to the appropriate genome database (human, mouse, rat, etc.) and eliminate from consideration any target sequences with significant homology to other coding sequences. Basically, BLAST, which can be found on the NCBI server at: www.ncbi.nlm.nih.gov/BLAST/, is used (Altschul S F et al., Nucleic Acids Res 1997 Sep. 1, 25(17): 3389-402).

3. Select qualifying target sequences for synthesis. Selecting several target sequences along the length of the gene to evaluate is typical.

Using the above protocol, the target sequence of the isolated double-stranded molecules of the present invention were designed as SEQ ID NO: 10 or 11.

Double-stranded molecules targeting the above-mentioned target sequences were respectively examined for their ability to suppress the growth of cells expressing the target genes. Therefore, the present invention provides double-stranded molecules targeting any one of the sequences selected from the group consisting of SEQ ID NO: 10 and 11.

The double-stranded molecule of the present invention may be directed to a single target Nectin-4 gene sequence or may be directed to a plurality of target Nectin-4 gene sequences.

A double-stranded molecule of the present invention targeting the above-mentioned targeting sequence of Nectin-4 gene include isolated polynucleotides that contain any of the nucleic acid sequences of target sequences and/or complementary sequences to the target sequences. Examples of polynucleotides targeting Nectin-4 gene include those containing the sequence of SEQ ID NO: 10 or 11 and/or complementary sequences to these nucleotides. However, the present invention is not limited to these examples, and minor modifications in the aforementioned nucleic acid sequences are acceptable so long as the modified molecule retains the ability to suppress the expression of Nectin-4 gene. Herein, the phrase “minor modification” as used in connection with a nucleic acid sequence indicates one, two or several substitution, deletion, addition or insertion of nucleic acids to the sequence.

In the context of the present invention, the term “several” as applies to nucleic acid substitutions, deletions, additions and/or insertions may mean 3 to 7, preferably 3 to 5, more preferably 3 to 4, even more preferably 3 nucleic acid residues.

According to the present invention, a double-stranded molecule of the present invention can be tested for its ability using the methods utilized in the Examples. In the Examples herein below, double-stranded molecules composed of sense strands of various portions of mRNA of Nectin-4 genes or antisense strands complementary thereto were tested in vitro for their ability to decrease production of Nectin-4 gene product in lung cancer cell lines (e.g., using NCI-H2170 or NCI-H358) according to standard methods. Furthermore, for example, reduction in Nectin-4 gene product in cells contacted with the candidate double-stranded molecule compared to cells cultured in the absence of the candidate molecule can be detected by, e.g. RT-PCR using primers for Nectin-4 mRNA mentioned under Example 1 item “Semi-quantitative RT-PCR”. Sequences which decrease the production of Nectin-4 gene product in vitro cell-based assays can then be tested for there inhibitory effects on cell growth. Sequences which inhibit cell growth in vitro cell-based assay can then be tested for their in vivo ability using animals with cancer, e.g. nude mouse xenograft models, to confirm decreased production of Nectin-4 product and decreased cancer cell growth.

When the isolated polynucleotide is RNA or derivatives thereof, base “t” should be replaced with “u” in the nucleotide sequences. As used herein, the term “complementary” refers to Watson-Crick or Hoogsteen base pairing between nucleotides units of a polynucleotide, and the term “binding” means the physical or chemical interaction between two polynucleotides. When the polynucleotide includes modified nucleotides and/or non-phosphodiester linkages, these polynucleotides may also bind each other as same manner. Generally, complementary polynucleotide sequences hybridize under appropriate conditions to form stable duplexes containing few or no mismatches. Furthermore, the sense strand and antisense strand of the isolated polynucleotide of the present invention can form double-stranded molecule or hairpin loop structure by the hybridization. In a preferred embodiment, such duplexes contain no more than 1 mismatch for every 10 matches. In an especially preferred embodiment, where the strands of the duplex are fully complementary, such duplexes contain no mismatches.

The polynucleotide is preferably less than 2744 nucleotides in length for Nectin-4. For example, the polynucleotide is less than 500, 200, 100, 75, 50, or 25 nucleotides in length for all of the genes. The isolated polynucleotides of the present invention are useful for forming double-stranded molecules against Nectin-4 gene or preparing template DNAs encoding the double-stranded molecules. When the polynucleotides are used for forming double-stranded molecules, the polynucleotide may be longer than 19 nucleotides, preferably longer than 21 nucleotides, and more preferably has a length of between about 19 and 25 nucleotides. Accordingly, the present invention provides the double-stranded molecules comprising a sense strand and an antisense strand, wherein the sense strand comprises a nucleotide sequence corresponding to a target sequence. In preferable embodiments, the sense strand hybridizes with antisense strand at the target sequence to form the double-stranded molecule having between 19 and 25 nucleotide pair in length.

The double-stranded molecules of the invention may contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which may be incorporated into the present molecules (WO03/070744; WO2005/045037). In one embodiment, modifications can be used to provide improved resistance to degradation or improved uptake. Examples of such modifications include, but are not limited to, phosphorothioate linkages, 2′-O-methyl ribonucleotides (especially on the sense strand of a double-stranded molecule), 2′-deoxy-fluoro ribonucleotides, 2′-deoxy ribonucleotides, “universal base” nucleotides, 5′-C— methyl nucleotides, and inverted deoxybasic residue incorporation (US20060122137).

In another embodiment, modifications can be used to enhance the stability or to increase targeting efficiency of the double-stranded molecule. Examples of such modifications include, but are not limited to, chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded molecule strand (WO2005/044976). For example, an unmodified pyrimidine nucleotide can be for a 2-thio, 5-alkynyl, 5-methyl, or 5-propynyl pyrimidine. Additionally, an unmodified purine can be substituted with a 7-diaza, 7-alkyl, or 7-alkenyl purine. In another embodiment, when the double-stranded molecule is a double-stranded molecule with a 3′ overhang, the 3′-terminal nucleotide overhanging nucleotides may be replaced by deoxyribonucleotides (Elbashir S M et al., Genes Dev 2001 Jan. 15, 15(2): 188-200). For further details, published documents such as US20060234970 are available. The present invention is not limited to these examples and any known chemical modifications may be employed for the double-stranded molecules of the present invention so long as the resulting molecule retains the ability to inhibit the expression of the target gene.

Furthermore, the double-stranded molecules of the invention may comprise both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule composed of a DNA strand (polynucleotide) and an RNA strand (polynucleotide), a chimera type double-stranded molecule containing both DNA and RNA on any or both of the single strands (polynucleotides), or the like may be formed for enhancing stability of the double-stranded molecule.

The hybrid of a DNA strand and an RNA strand may be either where the sense strand is DNA and the antisense strand is RNA, or the opposite so long as it can inhibit expression of the target gene when introduced into a cell expressing the gene. Preferably, the sense strand polynucleotide is DNA and the antisense strand polynucleotide is RNA. Also, the chimera type double-stranded molecule may be either where both of the sense and antisense strands are composed of DNA and RNA, or where any one of the sense and antisense strands is composed of DNA and RNA so long as it has an activity to inhibit expression of the target gene when introduced into a cell expressing the gene. In order to enhance stability of the double-stranded molecule, the molecule preferably contains as much DNA as possible, whereas to induce inhibition of the target gene expression, the molecule is required to be RNA within a range to induce sufficient inhibition of the expression.

As a preferred example of the chimera type double-stranded molecule, an upstream partial region (i.e., a region flanking to the target sequence or complementary sequence thereof within the sense or antisense strands) of the double-stranded molecule is RNA. Preferably, the upstream partial region indicates the 5′ side (5′-end) of the sense strand and the 3′ side (3′-end) of the antisense strand. Alternatively, regions flanking to 5′-end of sense strand and/or 3′-end of antisense strand are referred to upstream partial region. That is, in preferable embodiments, a region flanking to the 3′-end of the antisense strand, or both of a region flanking to the 5′-end of sense strand and a region flanking to the 3′-end of antisense strand are composed of RNA. For instance, the chimera or hybrid type double-stranded molecule of the present invention include following combinations.

sense strand:
5′-[---DNA---]-3′
3′-(RNA)-[DNA]-5′
:antisense strand,
sense strand:
5′-(RNA)-[DNA]-3′
3′-(RNA)-[DNA]-5′
:antisense strand,
and
sense strand:
5′-(RNA)-[DNA]-3′
3′-(---RNA---)-5′
:antisense strand.

The upstream partial region preferably is a domain composed of 9 to 13 nucleotides counted from the terminus of the target sequence or complementary sequence thereto within the sense or antisense strands of the double-stranded molecules. Moreover, preferred examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US20050004064).

In the present invention, the double-stranded molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA comprises the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

A loop sequence composed of an arbitrary nucleotide sequence can be located between the sense and antisense sequence in order to form the hairpin loop structure. Thus, the present invention also provides a double-stranded molecule having the general formula 5′-[A]-[B]-[A]-3′, wherein [A] is the sense strand containing a sequence corresponding to a target sequence, [B] is an intervening single-strand and [A] is the antisense strand containing a complementary sequence to [A]. The target sequence may be selected from among, for example, nucleotides of SEQ ID NO: 10 and 11.

The present invention is not limited to these examples, and the target sequence in [A] may be modified sequences from these examples so long as the double-stranded molecule retains the ability to suppress the expression of the targeted Nectin-4 gene. The region [A] hybridizes to [A′] to form a loop composed of the region [B]. The intervening single-stranded portion [B], i.e., loop sequence may be preferably 3 to 23 nucleotides in length. The loop sequence, for example, can be selected from among the following sequences (http://www.ambion.com/techlib/tb/tb_{—}506.html). Furthermore, loop sequence consisting of 23 nucleotides also provides active siRNA (Jacque J M et al., Nature 2002 Jul. 25, 418(6896): 435-8, Epub 2002 Jun. 26):

### CCC, CCACC, or CCACACC: Jacque J M et al., Nature 2002 Jul. 25, 418(6896): 435-8, Epub 2002 Jun. 26;

### UUCG: Lee N S et al., Nat Biotechnol 2002 May, 20(5): 500-5; Fruscoloni P et al., Proc Natl Acad Sci USA 2003 Feb. 18, 100(4): 1639-44, Epub 2003 Feb. 10; and UUCAAGAGA: Dykxhoom D M et al., Nat Rev Mol Cell Biol 2003 Jun., 4(6): 457-67.

Examples of preferred double-stranded molecules of the present invention having hairpin loop structure are shown below. In the following structure, the loop sequence can be selected from among AUG, CCC, UUCG, CCACC, CTCGAG, AAGCUU, CCACACC, and UUCAAGAGA; however, the present invention is not limited thereto:

ACAGUUACCACGUCUGAGG-[B]-CCUCAGACGUGGUAACUGU (for target sequence SEQ ID NO: 10); and

AAUGGUUCAUGGCCUGUUU-[B]-AAACAGGCCAUGAACCAUU (for target sequence SEQ ID NO: 11).

Furthermore, in order to enhance the inhibition activity of the double-stranded molecules, nucleotide “u” can be added to 3′ end of the sense strand and/or the antisense strand of the target sequence, as 3′ overhangs. The number of “u”s to be added is at least 2, generally 2 to 10, preferably 2 to 3. In cases where double-stranded molecules consists of a single polynucleotide to a hairpin loop structure, a 3′ overhang sequence may be added to the 3′ end of the single polynucleotide.

The method for preparing the double-stranded molecule is not particularly limited though it is preferable to use a chemical synthetic method known in the art. According to the chemical synthesis method, sense and antisense single-stranded polynucleotides are separately synthesized and then annealed together via an appropriate method to obtain a double-stranded molecule. Specific example for the annealing includes wherein the synthesized single-stranded polynucleotides are mixed in a molar ratio of preferably at least about 3:7, more preferably about 4:6, and most preferably substantially equimolar amount (i.e., a molar ratio of about 5:5). Next, the mixture is heated to a temperature at which double-stranded molecules dissociate and then is gradually cooled down. The annealed double-stranded polynucleotide can be purified by usually employed methods known in the art. Example of purification methods include methods utilizing agarose gel electrophoresis or wherein remaining single-stranded polynucleotides are optionally removed by, e.g., degradation with appropriate enzyme.

The regulatory sequences flanking Nectin-4 sequences may be identical or different, such that their expression can be modulated independently, or in a temporal or spatial manner. The double-stranded molecules can be transcribed intracellularly by cloning Nectin-4 gene templates into a vector containing, e.g., a RNA pol III transcription unit from the small nuclear RNA (snRNA) U6 or the human H1 RNA promoter.

Vectors Containing a Double-Stranded Molecule of the Present Invention:

Also included in the present invention are vectors containing one or more of the double-stranded molecules described herein, and a cell containing such a vector.

Of particular interest to the present invention are the following vectors of [1] to [10]:

[1] A vector, encoding a double-stranded molecule that, when introduced into a cell, inhibits in vivo expression of Nectin-4 and cell proliferation, such molecules composed of a sense strand and an antisense strand complementary thereto, hybridized to each other to form the double-stranded molecule.

[2]The vector of [1], wherein the sense strand contains a sequence corresponding to a target sequence of SEQ ID NO: 10 or 11;

[3]The vector of [2], encoding the double-stranded molecule, wherein the sense strand hybridizes with antisense strand at the target sequence to form the double-stranded molecule having less than about 100 nucleotide pair in length;

[4]The vector of [3], encoding the double-stranded molecule, wherein the sense strand hybridizes with antisense strand at the target sequence to form the double-stranded molecule having less than about 75 nucleotide pair in length;

[5]The vector of [4], encoding the double-stranded molecule, wherein the sense strand hybridizes with antisense strand at the target sequence to form the double-stranded molecule having less than about 50 nucleotide pair in length;

[6]The vector of [5] encoding the double-stranded molecule, wherein the sense strand hybridizes with antisense strand at the target sequence to form the double-stranded molecule having less than about 25 nucleotide pair in length;

[7]The vector of [6], encoding the double-stranded molecule, wherein the sense strand hybridizes with antisense strand at the target sequence to form the double-stranded molecule having between about 19 and about 25 nucleotide pair in length;

[8] The vector of [1], encoding the double-stranded molecule of claim 30, which has at least one 3′ overhang consisting of 2 or 3 nucleotides.

[9] The vector of [1], wherein the double-stranded molecule is composed of a single polynucleotide having both the sense and antisense strands linked by an intervening single-strand; and

[10] The vector of [9], encoding the double-stranded molecule having the general formula 5′4-[A]-[B]-[A′]-3′, wherein [A] is the sense strand, [B] is the intervening single-strand consisting of 3 to 23 nucleotides, and [A′] is the antisense strand comprising a complementary sequence to [A].

A vector of the present invention preferably encodes a double-stranded molecule of the present invention in an expressible form. Herein, the phrase “in an expressible form” indicates that the vector, when introduced into a cell, will express the molecule. In a preferred embodiment, the vector includes regulatory elements necessary for expression of the double-stranded molecule. Such vectors of the present invention may be used for producing the present double-stranded molecules, or directly as an active ingredient for treating cancer.

Vectors of the present invention can be produced, for example, by cloning Nectin-4 sequence into an expression vector so that regulatory sequences are operatively-linked to Nectin-4-sequence in a manner to allow expression (by transcription of the DNA molecule) of both strands (Lee N S et al., Nat Biotechnol 2002 May, 20(5): 500-5). For example, RNA molecule that is the antisense to mRNA is transcribed by a first promoter (e.g., a promoter sequence flanking to the 3′ end of the cloned DNA) and RNA molecule that is the sense strand to the mRNA is transcribed by a second promoter (e.g., a promoter sequence flanking to the 5′ end of the cloned DNA). The sense and antisense strands hybridize in vivo to generate a double-stranded molecule constructs for silencing of the gene. Alternatively, two vectors constructs respectively encoding the sense and antisense strands of the double-stranded molecule are utilized to respectively express the sense and anti sense strands and then forming a double-stranded molecule construct. Furthermore, the cloned sequence may encode a construct having a secondary structure (e.g., hairpin); namely, a single transcript of a vector contains both the sense and complementary antisense sequences of the target gene.

The vectors of the present invention may also be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivacaine, polymers, peptide-mediated) delivery, cationic lipid complexes, and particle-mediated (“gene gun”) or pressure-mediated delivery (see, e.g., U.S. Pat. No. 5,922,687).

The vectors of the present invention include, for example, viral or bacterial vectors. Examples of expression vectors include attenuated viral hosts, such as vaccinia or fowlpox (see, e.g., U.S. Pat. No. 4,722,848). This approach involves the use of vaccinia virus, e.g., as a vector to express nucleotide sequences that encode the double-stranded molecule. Upon introduction into a cell expressing the target gene, the recombinant vaccinia virus expresses the molecule and thereby suppresses the proliferation of the cell. Another example of useable vector includes Bacille Calmette Guerin (BCG). BCG vectors are described in Stover et al., Nature 1991, 351: 456-60. A wide variety of other vectors are useful for therapeutic administration and production of the double-stranded molecules; examples include adeno and adeno-associated virus vectors, retroviral vectors, Salmonella typhi vectors, detoxified anthrax toxin vectors, and the like. See, e.g., Shata et al., Mol Med Today 2000, 6: 66-71; Shedlock et al., J Leukoc Biol 2000, 68: 793-806; and Hipp et al., In Vivo 2000, 14: 571-85.

Methods of Inhibiting or Reducing Growth of a Cancer Cell and Treating Cancer Using a Double-Stranded Molecule of the Present Invention:

In the present invention, two different dsRNA were tested for their ability to inhibit cell growth. The two dsRNA effectively knocked down the expression of the gene in lung cancer cell lines coincided with suppression of cell proliferation (FIG. 4A).

Therefore, the present invention provides methods for inhibiting cancer cell growth, by inducing dysfunction of Nectin-4 gene via inhibiting the expression of Nectin-4. Nectin-4 gene expression can be inhibited by any of the aforementioned double-stranded molecules of the present invention which specifically target of Nectin-4 gene or the vectors of the present invention that can express any of the double-stranded molecules.

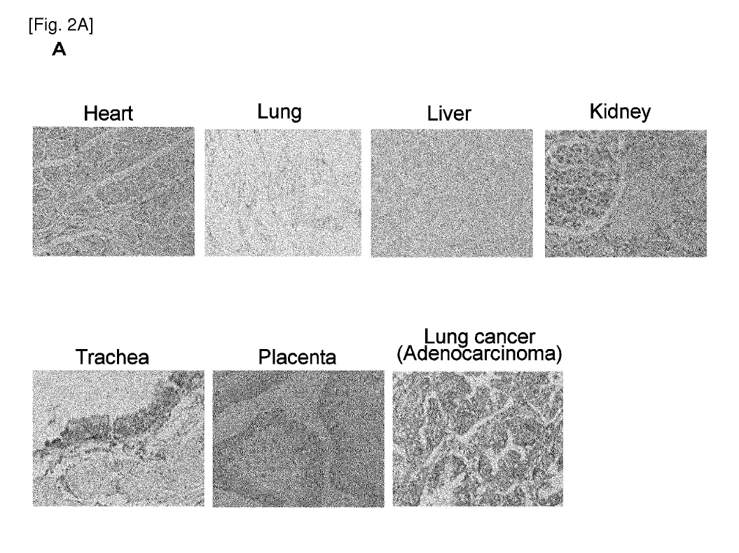

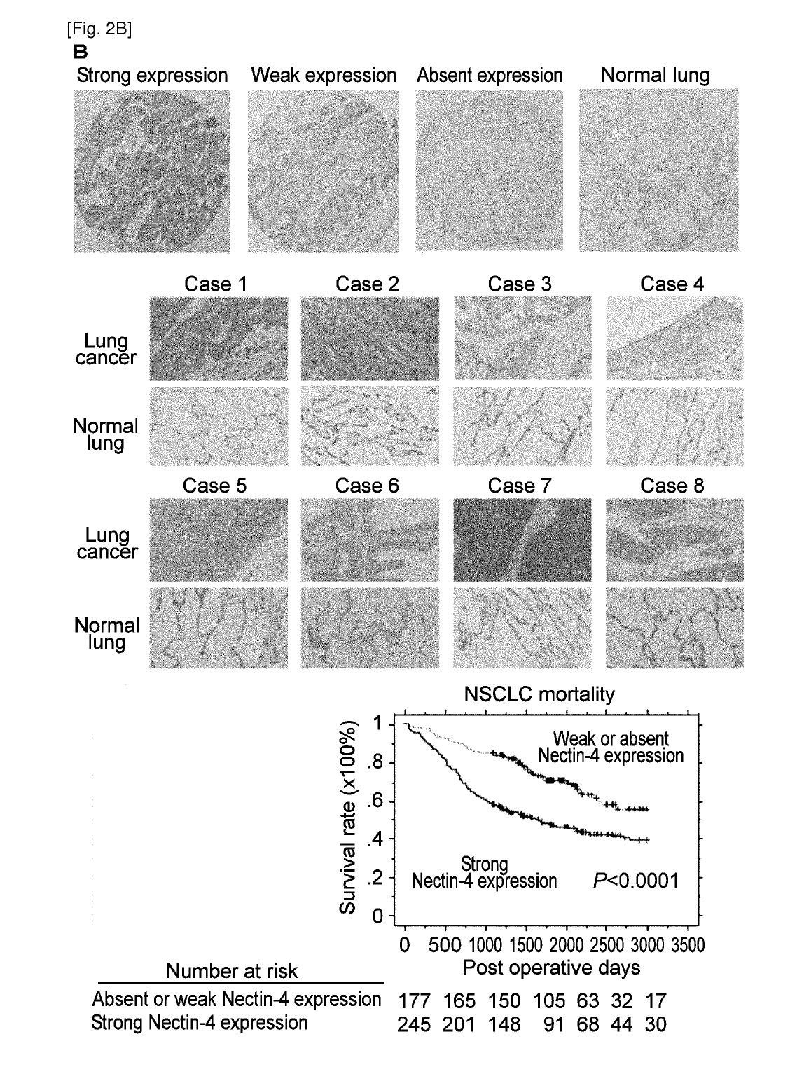

Such ability of the present double-stranded molecules and vectors to inhibit cell growth of cancerous cell indicates that they can be used for methods for treating cancer. Thus, the present invention provides methods to treat patients with lung cancer by administering a double-stranded molecule against Nectin-4 gene or a vector expressing the molecule without adverse effect because that gene were hardly detected in normal organs (FIG. 2A). In the present invention, the lung cancer is preferably non-small cell lung cancer (NSCLC).

Specifically, the present invention provides the following methods [1] to [22]:

[1] A method for inhibiting a growth of cancer cell or treating a cancer, wherein the cancer cell or the cancer expresses Nectin-4 gene, which method includes the step of administering at least one isolated double-stranded molecule which is composed of a sense strand and an antisense strand complementary thereto, hybridized to each other to form the double-stranded molecule, wherein the sense strand comprises the oligonucleotide corresponding to SEQ ID NO: 1 or fragment thereof;

[2] The double-stranded molecule of [1], wherein the sense strand contains the sequence corresponding to a target sequence selected from among SEQ ID NOs: 10 and 11;

[3] The method of [1], wherein the cancer to be treated is lung cancer;

[4] The method of [2], wherein the lung cancer is NSCLC;

[5] The method of [1], wherein plural kinds of the double-stranded molecules are administered at the same time;

[6] The method of [2], wherein the double-stranded molecule has a length of less than about 100 nucleotides;

[7] The method of [6], wherein the double-stranded molecule has a length of less than about 75 nucleotides;

[8] The method of [7], wherein the double-stranded molecule has a length of less than about 50 nucleotides;

[9] The method of [8], wherein the double-stranded molecule has a length of less than about 25 nucleotides;

[10] The method of [10], wherein the double-stranded molecule has a length of between about 19 and about 25 nucleotides in length;

[11] The method of [1], wherein the double-stranded molecule is composed of a single polynucleotide containing both the sense strand and the antisense strand linked by an intervening single-strand;

[12] The method of [11], wherein the double-stranded molecule has the general formula 5′-[A]-[B]-[A′]-3′, wherein [A] is the sense strand containing a sequence corresponding to a target sequence selected from among SEQ ID NOs: 10 and 11, [B] is the intervening single strand composed of 3 to 23 nucleotides, and [A′] is the antisense strand containing a sequence complementary to [A];

[13] The method of [1], wherein the double-stranded molecule is an RNA;

[14] The method of [1], wherein the double-stranded molecule contains both DNA and RNA;

[15] The method of [14], wherein the double-stranded molecule is a hybrid of a DNA polynucleotide and an RNA polynucleotide;

[16] The method of [15] wherein the sense and antisense strand polynucleotides are composed of DNA and RNA, respectively;

[17] The method of [15], wherein the double-stranded molecule is a chimera of DNA and RNA;

[18] The method of [17], wherein a region flanking to the 3′-end of the antisense strand, or both of a region flanking to the 5′-end of sense strand and a region flanking to the 3′-end of antisense strand are composed of RNA;

[19] The method of [18], wherein the flanking region is composed of 9 to 13 nucleotides;

[20] The method of [1], wherein the double-stranded molecule contains 3′ overhangs;

[21] A method for inhibiting a growth of cancer cell or treating a cancer, wherein the cancer cell or the cancer expresses Nectin-4 gene, which method includes the step of administering at least one isolated vector which encode the double-stranded molecule described in [1]; and

[22] The method of [1] and [21], wherein the double-stranded molecule or vector is contained in a composition which includes, in addition to the molecule, a transfection-enhancing agent and pharmaceutically acceptable carrier.

Further, exogenous overexpression of Nectin-4 led to enhancement of tumor growth and cell invasion (FIGS. 4B-F), and it is demonstrated that Nectin-4 involved in cell invasion. Therefore, it is expected to inhibit cancer cell invasion by inducing dysfunction of Nectin-4 gene. Thus, the present invention also provide methods for inhibiting cancer cell invasion, comprising the step of administering at least one isolated double-stranded molecule of the present invention or vector encoding thereof to a subject.

The methods of the present invention may be preferably applicable to lung cancer, bladder cancer and cervical carcinoma, more preferably lung cancer, further more preferably NSCLCs.

The method of the present invention will be described in more detail below.

The growth of cells expressing Nectin-4 gene may be inhibited by contacting the cells with a double-stranded molecule against Nectin-4 gene, a vector expressing the molecule or a composition containing the same. The cell may be further contacted with a transfection agent. Suitable transfection agents are known in the art. The phrase “inhibition of cell growth” indicates that the cell proliferates at a lower rate or has decreased viability as compared to a cell not exposed to the molecule. Cell growth may be measured by methods known in the art, e.g., using the MTT cell proliferation assay.

The growth of any kind of cell may be suppressed according to the present method so long as the cell expresses or over-expresses Nectin-4 gene. Exemplary cells include lung cancer cells, especially, NSCLC.

Thus, patients suffering from or at risk of developing disease related to Nectin-4 may be treated by administering at least one of the present double-stranded molecules, at least one vector expressing at least one of the molecules or at least one composition containing at least one of the molecules. For example, patients of lung cancer may be treated according to the present methods. The type of cancer may be identified by standard methods according to the particular type of tumor to be diagnosed. Lung cancer may be diagnosed, for example, with CEA, CYFRA and so on, as lung cancer marker, or with Chest X-Ray and/or Sputum Cytology. More preferably, patients treated by the methods of the present invention are selected by detecting the expression of Nectin-4 in a biopsy from the patient by RT-PCR or immunoassay. Preferably, before the treatment of the present invention, the biopsy specimen from the subject is confirmed for Nectin-4 gene over-expression by methods known in the art, for example, immunohistochemical analysis or RT-PCR.

According to the present method to inhibit cell growth and thereby treating a cancer, when administering plural kinds of the double-stranded molecules (or vectors expressing or compositions containing the same).

For inhibiting cell growth, a double-stranded molecule of present invention may be directly introduced into the cells in a form to achieve binding of the molecule with corresponding mRNA transcripts. Alternatively, as described above, a DNA encoding the double-stranded molecule may be introduced into cells as a vector. For introducing the double-stranded molecules and vectors into the cells, transfection-enhancing agent, such as FuGENE (Roche diagnostics), Lipofectamine 2000 (Invitrogen), Oligo-fectamine (Invitrogen), and Nucleofector (Wako pure Chemical), may be employed.

A treatment is deemed “efficacious” if it leads to clinical benefit such as, reduction in expression of Nectin-4-gene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

It is understood that the double-stranded molecule of the invention degrades the Nectin-4 mRNA in substoichiometric amounts. Without wishing to be bound by any theory, it is believed that the double-stranded molecule of the invention causes degradation of the target mRNA in a catalytic manner. Thus, compared to standard cancer therapies, significantly less a double-stranded molecule needs to be delivered at or near the site of cancer to exert therapeutic effect.

One skilled in the art can readily determine an effective amount of the double-stranded molecule of the invention to be administered to a given subject, by taking into account factors such as body weight, age, sex, type of disease, symptoms and other conditions of the subject; the route of administration; and whether the administration is regional or systemic. Generally, an effective amount of the double-stranded molecule of the invention is an intercellular concentration at or near the cancer site of from about 1 nanomolar (nM) to about 100 nM, preferably from about 2 nM to about 50 nM, more preferably from about 2.5 nM to about 10 nM. It is contemplated that greater or smaller amounts of the double-stranded molecule can be administered. The precise dosage required for a particular circumstance may be readily and routinely determined by one of skill in the art.

The present methods can be used to inhibit the growth or metastasis of cancer expressing Nectin-4; for example lung cancer, especially NSCLC. In particular, a double-stranded molecule containing a target sequence of Nectin-4 (i.e., SEQ ID NOs: 10 or 11) is particularly preferred for the treatment of lung cancer.

For treating cancer, the double-stranded molecule of the invention can also be administered to a subject in combination with a pharmaceutical agent different from the double-stranded molecule. Alternatively, the double-stranded molecule of the invention can be administered to a subject in combination with another therapeutic method designed to treat cancer. For example, the double-stranded molecule of the invention can be administered in combination with therapeutic methods currently employed for treating cancer or preventing cancer metastasis (e.g., radiation therapy, surgery and treatment using chemotherapeutic agents).

In the present methods, the double-stranded molecule can be administered to the subject either as a naked double-stranded molecule, in conjunction with a delivery reagent, or as a recombinant plasmid or viral vector which expresses the double-stranded molecule.

Suitable delivery reagents for administration in conjunction with the present a double-stranded molecule include the Mirus Transit TKO lipophilic reagent; lipofectin; lipofectamine; cellfectin; or polycations (e.g., polylysine), or liposomes. A preferred delivery reagent is a liposome.

Liposomes can aid in the delivery of the double-stranded molecule to a particular tissue, such as retinal or tumor tissue, and can also increase the blood half-life of the double-stranded molecule. Liposomes suitable for use in the invention are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al., Ann Rev Biophys Bioeng 1980, 9: 467; and U.S. Pat. Nos. 4,235,871; 4,501,728; 4,837,028; and 5,019,369, the entire disclosures of which are herein incorporated by reference.

Preferably, the liposomes encapsulating the present double-stranded molecule comprises a ligand molecule that can deliver the liposome to the cancer site. Ligands which bind to receptors prevalent in tumor or vascular endothelial cells, such as monoclonal antibodies that bind to tumor antigens or endothelial cell surface antigens, are preferred.

Particularly preferably, the liposomes encapsulating the present double-stranded molecule are modified so as to avoid clearance by the mononuclear macrophage and reticuloendothelial systems, for example, by having opsonization-inhibition moieties bound to the surface of the structure. In one embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example, solid tumors, will efficiently accumulate these liposomes; see Gabizon et al., Proc Natl Acad Sci USA 1988, 18: 6949-53. In addition, the reduced uptake by the RES lowers the toxicity of stealth liposomes by preventing significant accumulation in liver and spleen. Thus, liposomes of the invention that are modified with opsonization-inhibition moieties can deliver the present double-stranded molecule to tumor cells.

Opsonization inhibiting moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as poly-acrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM₁. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

Preferably, the opsonization-inhibiting moiety is a PEG, PPG, or derivatives thereof. Liposomes modified with PEG or PEG-derivatives are sometimes called “PEGylated liposomes”.

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture such as tetrahydrofuran and water in a 30:12 ratio at 60 degrees C.

Vectors expressing a double-stranded molecule of the invention are discussed above. Such vectors expressing at least one double-stranded molecule of the invention can also be administered directly or in conjunction with a suitable delivery reagent, including the Mirus Transit LT1 lipophilic reagent; lipofectin; lipofectamine; cellfectin; polycations (e.g., polylysine) or liposomes. Methods for delivering recombinant viral vectors, which express a double-stranded molecule of the invention, to an area of cancer in a patient are within the skill of the art.

The double-stranded molecule of the invention can be administered to the subject by any means suitable for delivering the double-stranded molecule into cancer sites. For example, the double-stranded molecule can be administered by gene gun, electroporation, or by other suitable parenteral or enteral administration routes.

Suitable enteral administration routes include oral, rectal, or intranasal delivery.

Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection); subcutaneous injection or deposition including subcutaneous infusion (such as by osmotic pumps); direct application to the area at or near the site of cancer, for example by a catheter or other placement device (e.g., a suppository or an implant comprising a porous, non-porous, or gelatinous material); and inhalation. It is preferred that injections or infusions of the double-stranded molecule or vector be given at or near the site of cancer.

The double-stranded molecule of the invention can be administered in a single dose or in multiple doses. Where the administration of the double-stranded molecule of the invention is by infusion, the infusion can be a single sustained dose or can be delivered by multiple infusions. Injection of the agent directly into the tissue is at or near the site of cancer preferred. Multiple injections of the agent into the tissue at or near the site of cancer are particularly preferred.

One skilled in the art can also readily determine an appropriate dosage regimen for administering the double-stranded molecule of the invention to a given subject. For example, the double-stranded molecule can be administered to the subject once, for example, as a single injection or deposition at or near the cancer site. Alternatively, the double-stranded molecule can be administered once or twice daily to a subject for a period of from about three to about twenty-eight days, more preferably from about seven to about ten days. In a preferred dosage regimen, the double-stranded molecule is injected at or near the site of cancer once a day for seven days. Where a dosage regimen comprises multiple administrations, it is understood that the effective amount of a double-stranded molecule administered to the subject can comprise the total amount of a double-stranded molecule administered over the entire dosage regimen.

Compositions Containing a Double-Stranded Molecule of the Present Invention:

In addition to the above, the present invention also provides pharmaceutical compositions that include at least one of the present double-stranded molecules or the vectors coding for the molecules. Specifically, the present invention provides the following compositions [1] to [22]:

[1] A composition for inhibiting a growth of cancer cell or treating a cancer, wherein the cancer cell and the cancer expresses Nectin-4 gene, including at least one isolated double-stranded molecule inhibiting the expression of Nectin-4 and the cell proliferation, which molecule is composed of a sense strand and an antisense strand complementary thereto, hybridized to each other to form the double-stranded molecule, wherein the sense strand comprises the oligonucleotide corresponding to SEQ ID NO: 1 or fragment thereof;

[2] The composition of [1], wherein the double-stranded molecule, wherein the sense strand contains a sequence corresponding to a target sequence selected from among SEQ ID NOs: 10 and 11;

[3] The method of [1], wherein the cancer to be treated is lung cancer;

[4] The method of [3], wherein the lung cancer is NSCLC;

[5] The composition of [1], wherein the composition contains plural kinds of the double-stranded molecules;

[6] The composition of [2], wherein the double-stranded molecule has a length of less than about 100 nucleotides;

[7] The composition of [6], wherein the double-stranded molecule has a length of less than about 75 nucleotides;

[8] The composition of [7], wherein the double-stranded molecule has a length of less than about 50 nucleotides;

[9] The composition of [8], wherein the double-stranded molecule has a length of less than about 25 nucleotides;

[10] The composition of [9], wherein the double-stranded molecule has a length of between about 19 and about 25 nucleotides;

[11] The composition of [1], wherein the double-stranded molecule is composed of a single polynucleotide containing the sense strand and the antisense strand linked by an intervening single-strand;

[12] The composition of [11], wherein the double-stranded molecule has the general formula 5′-[A]-[B]-[A′]-3′, wherein [A] is the sense strand sequence contains a sequence corresponding to a target sequence selected from among SEQ ID NOs: 10 and 11, [B] is the intervening single-strand consisting of 3 to 23 nucleotides, and [A′] is the antisense strand contains a sequence complementary to [A];

[13] The composition of [1], wherein the double-stranded molecule is an RNA;

[14] The composition of [1], wherein the double-stranded molecule is DNA and/or RNA;

[15] The composition of [14], wherein the double-stranded molecule is a hybrid of a DNA polynucleotide and an RNA polynucleotide;

[16] The composition of [15], wherein the sense and antisense strand polynucleotides are composed of DNA and RNA, respectively;

[17] The composition of [14], wherein the double-stranded molecule is a chimera of DNA and RNA;

[18] The composition of [17], wherein a region flanking to the 3′-end of the antisense strand, or both of a region flanking to the 5′-end of sense strand and a region flanking to the 3′-end of antisense strand are composed of RNA;

[19] The composition of [18], wherein the flanking region is composed of 9 to 13 nucleotides;

[20] The composition of [1], wherein the double-stranded molecule contains 3′ overhangs;

[21] A composition for inhibiting a growth of cancer cell or treating a cancer, wherein the cancer cell and the cancer expresses Nectin-4 gene, wherein the composition includes at least one isolated vector which encodes the double-stranded molecule described in [1] and contained in the composition; and

[22] The composition of [1] an [21], wherein the composition includes a transfection-enhancing agent and pharmaceutically acceptable carrier.

The composition of the present invention may be also applied to inhibiting cancer cell invasion. Preferably, the composition of the present invention may be applied to lung cancer, bladder cancer and cervical carcinoma, more preferably lung cancer, further more preferably NSCLCs.

Suitable compositions of the present invention are described in additional detail below.

The double-stranded molecules of the invention are preferably formulated as pharmaceutical compositions prior to administering to a subject, according to techniques known in the art. Pharmaceutical compositions of the present invention are characterized as being at least sterile and pyrogen-free. As used herein, “pharmaceutical compositions” include compositions for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical compositions contain at least one of the double-stranded molecules or vectors encoding them of the present invention (e.g., 0.1 to 90% by weight), or a physiologically acceptable salt of the molecule, mixed with a physiologically acceptable carrier medium. Preferred physiologically acceptable carrier media are water, buffered water, normal saline, 0.4% saline, 0.3% glycine, hyaluronic acid and the like.

According to the present invention, the composition may contain plural kinds of the double-stranded molecules, each of the molecules may be directed to Nectin-4.

Furthermore, the present composition may contain a vector coding for one or plural double-stranded molecules. For example, the vector may encode one or two kinds of the present double-stranded molecules. Alternatively, the present composition may contain plural kinds of vectors, each of the vectors coding for a different double-stranded molecule.

Moreover, the present double-stranded molecules may be contained as liposomes in the present composition. The details of liposomes are described above.

Pharmaceutical compositions of the invention can also include conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can include any of the carriers and excipients listed above and 10-95%, preferably 25-75%, of one or more double-stranded molecule of the invention. A pharmaceutical composition for aerosol (inhalational) administration can comprise 0.01-20% by weight, preferably 1-10% by weight, of one or more double-stranded molecule of the invention encapsulated in a liposome as described above, and propellant. A carrier can also be included as desired; e.g., lecithin for intranasal delivery.

In addition to the above, the present composition may contain other pharmaceutical active ingredients so long as they do not inhibit the in vivo function of the present double-stranded molecules. For example, the composition may contain chemotherapeutic agents conventionally used for treating cancers.

In another embodiment, the present invention also provides the use of the double-stranded nucleic acid molecules of the present invention in manufacturing a pharmaceutical composition for treating a lung cancer characterized by the expression of Nectin-4. For example, the present invention relates to a use of double-stranded nucleic acid molecule inhibiting the expression of gene selected from among Nectin-4 in a cell, which molecule includes a sense strand and an antisense strand complementary thereto, hybridized to each other to form the double-stranded nucleic acid molecule and targets to a sequence selected from among SEQ ID NOs: 10 and 11, for manufacturing a pharmaceutical composition for treating lung cancer expressing Nectin-4.

Alternatively, the present invention further provides a method or process for a pharmaceutical composition for treating a lung cancer characterized by the expression of Nectin-4, wherein the method or process includes a step for formulating a pharmaceutically or physiologically acceptable carrier with a double-stranded nucleic acid molecule inhibiting the expression of Nectin-4 in a cell, which over-expresses the gene, which molecule includes a sense strand and an antisense strand complementary thereto, hybridized to each other to form the double-stranded nucleic acid molecule and targets to a sequence selected from among SEQ ID NOs: 10 and 11 as active ingredients.

In another embodiment, the present invention also provides a method or process for manufacturing a pharmaceutical composition for treating a lung cancer characterized by the expression of Nectin-4, wherein the method or process includes a step for admixing an active ingredient with a pharmaceutically or physiologically acceptable carrier, wherein the active ingredient is a double-stranded nucleic acid molecule inhibiting the expression of Nectin-4 in a cell, which over-expresses the gene, which molecule includes a sense strand and an antisense strand complementary thereto, hybridized to each other to form the double-stranded nucleic acid molecule and targets to a sequence selected from among SEQ ID NOs: 10 and 11.

Screening for an Anti-Lung Cancer Compound:

In the context of the present invention, agents to be identified through the present screening methods may be any compound or composition including several compounds. Furthermore, the test agent exposed to a cell or protein according to the screening methods of the present invention may be a single compound or a combination of compounds. When a combination of compounds is used in the methods, the compounds may be contacted sequentially or simultaneously.

The compound screened by the present screening method may be suitable candidate compound for treating and/or preventing cancer, inhibiting cancer cell growth and/or cancer cell invasion. In the present invention, cancer is associated with Nectin-4 over-expression. Accordingly, the screened compounds may be preferably applied to the cancers correlated or associated with Nectin-4 overexpression. In the preferable embodiments, the cancers correlated or associated with Nectin-4 overexpression are lung cancer, bladder cancer and cervical carcinoma, more preferably lung cancer, further more preferably NSCLCs.

Any test agent, for example, cell extracts, cell culture supernatant, products of fermenting microorganism, extracts from marine organism, plant extracts, purified or crude proteins, peptides, non-peptide compounds, synthetic micromolecular compounds (including nucleic acid constructs, such as antisense RNA, siRNA, Ribozymes, and aptamer etc or antibody.) and natural compounds can be used in the screening methods of the present invention. The test agent of the present invention can be also obtained using any of the numerous approaches in combinatorial library methods known in the art, including (1) biological libraries, (2) spatially addressable parallel solid phase or solution phase libraries, (3) synthetic library methods requiring deconvolution, (4) the “one-bead one-compound” library method and (5) synthetic library methods using affinity chromatography selection. The biological library methods using affinity chromatography selection is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of compounds may be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. Nos. 5,571,698; 5,403,484, and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002103360).

A compound in which a part of the structure of the compound screened by any of the present screening methods is converted by addition, deletion and/or replacement, is included in the agents obtained by the screening methods of the present invention.

Furthermore, when the screened test agent is a protein, for obtaining a DNA encoding the protein, either the whole amino acid sequence of the protein may be determined to deduce the nucleic acid sequence coding for the protein, or partial amino acid sequence of the obtained protein may be analyzed to prepare an oligo DNA as a probe based on the sequence, and screen cDNA libraries with the probe to obtain a DNA encoding the protein. The obtained DNA is confirmed it's usefulness in preparing the test agent which is a candidate for treating or preventing cancer.

Test agents useful in the screenings described herein can also be antibodies that specifically bind to Nectin-4 protein or partial peptides thereof that lack the biological activity of the original proteins in vivo.

Although the construction of test agent libraries is well known in the art, herein below, additional guidance in identifying test agents and construction libraries of such agents for the present screening methods are provided.

(i) Molecular Modeling:

Construction of test agent libraries is facilitated by knowledge of the molecular structure of compounds known to have the properties sought, and/or the molecular structure of the target molecules to be inhibited, i.e., Nectin-4 protein. One approach to preliminary screening of test agents suitable for further evaluation is computer modeling of the interaction between the test agent and Nectin-4 protein.

Computer modeling technology allows the visualization of the three-dimensional atomic structure of a selected molecule and the rational design of new compounds that will interact with the molecule. The three-dimensional construct typically depends on data from x-ray crystallographic analysis or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modeling system described generally above includes the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen et al. Acta Pharmaceutica Fennica 1988, 97: 159-66; Ripka, New Scientist 1988, 54-8; McKinlay & Rossmann, Annu Rev Pharmacol Toxiciol 1989, 29: 111-22; Perry & Davies, Prog Clin Biol Res 1989, 291: 189-93; Lewis & Dean, Proc R Soc Lond 1989, 236: 125-40, 141-62; and, with respect to a model receptor for nucleic acid components, Askew et al., J Am Chem Soc 1989, 111: 1082-90.

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. See, e.g., DesJarlais et al., Med Chem 1988, 31: 722-9; Meng et al., J Computer Chem 1992, 13: 505-24; Meng et al., Proteins 1993, 17: 266-78; Shoichet et al., Science 1993, 259: 1445-50.

Once a putative inhibitor has been identified, combinatorial chemistry techniques can be employed to construct any number of variants based on the chemical structure of the identified putative inhibitor, as detailed below. The resulting library of putative inhibitors, or “test agents” may be screened using the methods of the present invention to identify test agents treating or preventing the lung cancer.

(ii) Combinatorial Chemical Synthesis:

Combinatorial libraries of test agents may be produced as part of a rational drug design program involving knowledge of core structures existing in known inhibitors. This approach allows the library to be maintained at a reasonable size, facilitating high throughput screening. Alternatively, simple, particularly short, polymeric molecular libraries may be constructed by simply synthesizing all permutations of the molecular family making up the library. An example of this latter approach would be a library of all peptides six amino acids in length. Such a peptide library could include every 6 amino acid sequence permutation. This type of library is termed a linear combinatorial chemical library.

Preparation of Combinatorial Chemical Libraries is Well Known to Those of Skill in the art, and may be generated by either chemical or biological synthesis. Combinatorial chemical libraries include, but are not limited to, peptide libraries (see, e.g., U.S. Pat. No. 5,010,175; Furka, Int J Pept Prot Res 1991, 37: 487-93; Houghten et al., Nature 1991, 354: 84-6). Other chemistries for generating chemical diversity libraries can also be used. Such chemistries include, but are not limited to: peptides (e.g., PCT Publication No. WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (DeWitt et al., Proc Natl Acad Sci USA 1993, 90:6909-13), vinylogous polypeptides (Hagihara et al., J Amer Chem Soc 1992, 114: 6568), nonpeptidal peptidomimetics with glucose scaffolding (Hirschmann et al., J Amer Chem Soc 1992, 114: 9217-8), analogous organic syntheses of small compound libraries (Chen et al., J. Amer Chem Soc 1994, 116: 2661), oligocarbamates (Cho et al., Science 1993, 261: 1303), and/or peptidylphosphonates (Campbell et al., J Org Chem 1994, 59: 658), nucleic acid libraries (see Ausubel, Current Protocols in Molecular Biology 1995 supplement; Sambrook et al., Molecular Cloning: A Laboratory Manual, 1989, Cold Spring Harbor Laboratory, New York, USA), peptide nucleic acid libraries (see, e.g., U.S. Pat. No. 5,539,083), antibody libraries (see, e.g., Vaughan et al., Nature Biotechnology 1996, 14(3):309-14 and PCT/US96/10287), carbohydrate libraries (see, e.g., Liang et al., Science 1996, 274: 1520-22; U.S. Pat. No. 5,593,853), and small organic molecule libraries (see, e.g., benzodiazepines, Gordon E M. Curr Opin Biotechnol. 1995 Dec. 1; 6(6):624-31; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

(iii) Phage Display:

Another approach uses recombinant bacteriophage to produce libraries. Using the “phage method” (Scott & Smith, Science 1990, 249: 386-90; Cwirla et al., Proc Natl Acad Sci USA 1990, 87: 6378-82; Devlin et al., Science 1990, 249: 404-6), very large libraries can be constructed (e.g., 106-108 chemical entities). A second approach uses primarily chemical methods, of which the Geysen method (Geysen et al., Molecular Immunology 1986, 23: 709-15; Geysen et al., J Immunologic Method 1987, 102: 259-74); and the method of Fodor et al. (Science 1991, 251: 767-73) are examples. Furka et al. (14th International Congress of Biochemistry 1988, Volume #5, Abstract FR:013; Furka, Int J Peptide Protein Res 1991, 37: 487-93), Houghten (U.S. Pat. No. 4,631,211) and Rutter et al. (U.S. Pat. No. 5,010,175) describe methods to produce a mixture of peptides that can be tested as agonists or antagonists.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Screening for a Nectin-4 Binding Compound:

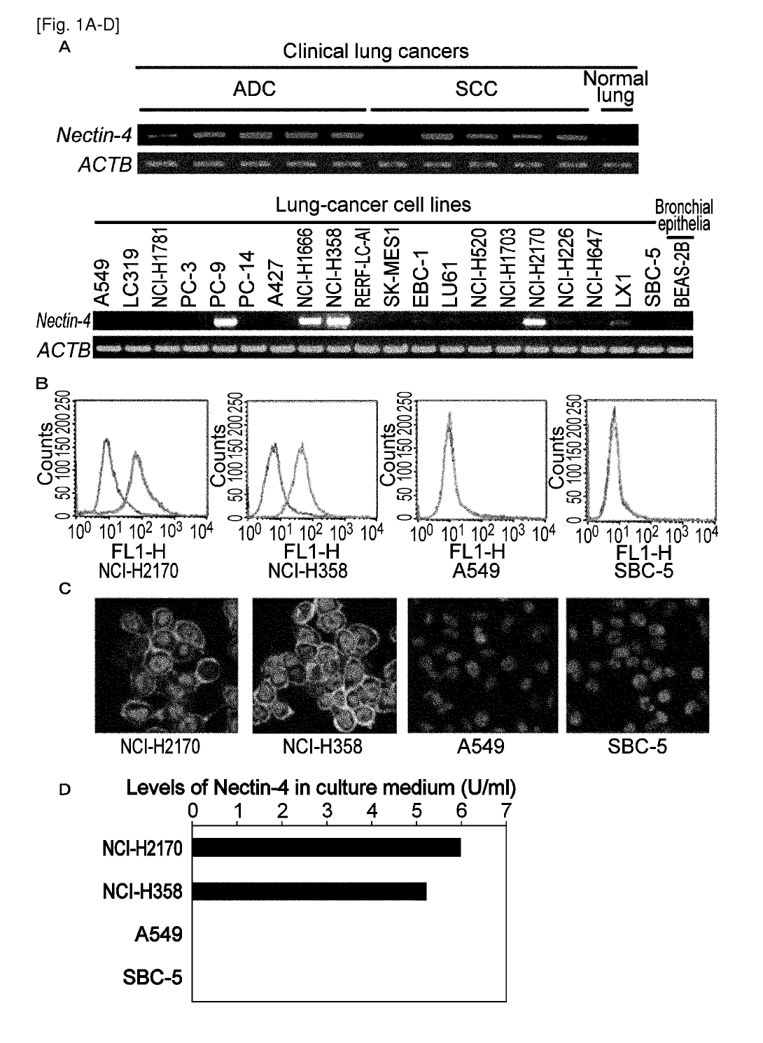

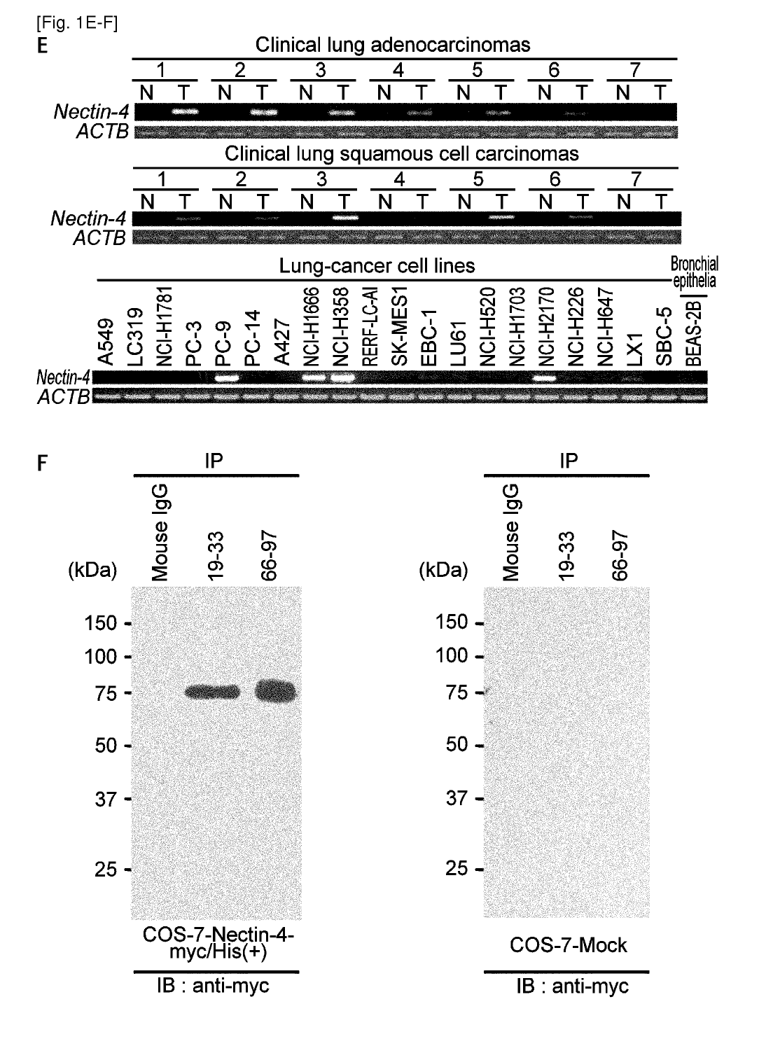

In present invention, over-expression of Nectin-4 was detected in lung cancer, in spite of no expression in normal organs (FIGS. 1, and 2A). Therefore, using the Nectin-4 genes, proteins encoded by the genes, the present invention provides a method of screening for a compound that binds to Nectin-4. Due to the expression of Nectin-4 in cancer, a compound binds to Nectin-4 is expected to suppress the proliferation of cancer cells, and thus be useful for treating or preventing cancer. Therefore, the present invention also provides a method for screening a candidate compound that suppresses the proliferation of cancer cells, and a method for screening a compound for treating or preventing cancer using the Nectin-4 polypeptide. Specially, an embodiment of this screening method includes the steps of:

(a) contacting a test compound with a polypeptide encoded by a polynucleotide of Nectin-4;

(b) detecting the binding activity between the polypeptide and the test compound; and

(c) selecting the test compound that binds to the polypeptide.

In the context of the present invention, the therapeutic effect may be correlated with the binding level of the test agent or compound and Nectin-4 protein(s). For example, when the test agent or compound binds to a Nectin-4 protein, the test agent or compound may identified or selected as a candidate agent or compound having the requisite therapeutic effect. Alternatively, when the test agent or compound does not binds to Nectin-4 proteins, the test agent or compound may identified as the agent or compound having no significant therapeutic effect.

The method of the present invention will be described in more detail below.

The Nectin-4 polypeptide to be used for screening may be a recombinant polypeptide or a protein derived from the nature or a partial peptide thereof. The polypeptide to be contacted with a test compound can be, for example, a purified polypeptide, a soluble protein, a form bound to a carrier or a fusion protein fused with other polypeptides.

As a method of screening for proteins, for example, that bind to the Nectin-4 polypeptide using the Nectin-4 polypeptide, many methods well known by a person skilled in the art can be used. Such a screening can be conducted by, for example, immunoprecipitation method, specifically, in the following manner. The gene encoding the Nectin-4 polypeptide is expressed in host (e.g., animal) cells and so on by inserting the gene to an expression vector for foreign genes, such as pSV2neo, pcDNA I, pcDNA3.1, pCAGGS and pCD8.

The promoter to be used for the expression may be any promoter that can be used commonly and include, for example, the SV40 early promoter (Rigby in Williamson (ed.), Genetic Engineering, vol. 3. Academic Press, London, 83-141 (1982)), the EF-alpha promoter (Kim et al., Gene 91: 217-23 (1990)), the CAG promoter (Niwa et al., Gene 108: 193 (1991)), the RSV LTR promoter (Cullen, Methods in Enzymology 152: 684-704 (1987)) the SR alpha promoter (Takebe et al., Mol Cell Biol 8: 466 (1988)), the CMV immediate early promoter (Seed and Aruffo, Proc Natl Acad Sci USA 84: 3365-9 (1987)), the SV40 late promoter (Gheysen and Fiers, J Mol Appl Genet. 1: 385-94 (1982)), the Adenovirus late promoter (Kaufman et al., Mol Cell Biol 9: 946 (1989)), the HSV TK promoter and so on.

The introduction of the gene into host cells to express a foreign gene can be performed according to any methods, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard B., Cell 76: 1025-37 (1994); Lamb et al., Nature Genetics 5: 22-30 (1993): Rabindran et al., Science 259: 230-4 (1993)) and so on.

The polypeptide encoded by Nectin-4 gene can be expressed as a fusion protein including a recognition site (epitope) of a monoclonal antibody by introducing the epitope of the monoclonal antibody, whose specificity has been revealed, to the N- or C-terminus of the polypeptide. A commercially available epitope-antibody system can be used (Experimental Medicine 13: 85-90 (1995)). Vectors which can express a fusion protein with, for example, beta-galactosidase, maltose binding protein, glutathione S-transferase, green florescence protein (GFP) and so on by the use of its multiple cloning sites are commercially available. Also, a fusion protein prepared by introducing only small epitopes consisting of several to a dozen amino acids so as not to change the property of the Nectin-4 polypeptide by the fusion is also reported. Epitopes, such as polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and monoclonal antibodies recognizing them can be used as the epitope-antibody system for screening proteins binding to the Nectin-4 polypeptide (Experimental Medicine 13: 85-90 (1995)).

In immunoprecipitation, an immune complex is formed by adding these antibodies to cell lysate prepared using an appropriate detergent. The immune complex consists of the Nectin-4 polypeptide, a polypeptide including the binding ability with the polypeptide, and an antibody. Immunoprecipitation can be also conducted using antibodies against the Nectin-4 polypeptide, besides using antibodies against the above epitopes. An immune complex can be precipitated, for example by Protein A sepharose or Protein G sepharose when the antibody is a mouse IgG antibody. If the polypeptide encoded by Nectin-4 gene is prepared as a fusion protein with an epitope, such as GST, an immune complex can be formed in the same manner as in the use of the antibody against the Nectin-4 polypeptide, using a substance specifically binding to these epitopes, such as glutathione-Sepharose 4B.

Immunoprecipitation can be performed by following or according to, for example, the methods in the literature (Harlow and Lane, Antibodies, 511-52, Cold Spring Harbor Laboratory publications, New York (1988)).

SDS-PAGE is commonly used for analysis of immunoprecipitated proteins and the bound protein can be analyzed by the molecular weight of the protein using gels with an appropriate concentration. Since the protein bound to the Nectin-4 polypeptide is difficult to detect by a common staining method, such as Coomassie staining or silver staining, the detection sensitivity for the protein can be improved by culturing cells in culture medium containing radioactive isotope, ³⁵S-methionine or ³⁵S-cystein, labeling proteins in the cells, and detecting the proteins. The target protein can be purified directly from the SDS-polyacrylamide gel and its sequence can be determined, when the molecular weight of a protein has been revealed.

As a method of screening for proteins binding to the Nectin-4 polypeptide using the polypeptide, for example, West-Western blotting analysis (Skolnik et al., Cell 65: 83-90 (1991)) can be used. Specifically, a protein binding to the Nectin-4 polypeptide can be obtained by preparing a cDNA library from cultured cells expected to express a protein binding to the Nectin-4 polypeptide using a phage vector (e.g., ZAP), expressing the protein on LB-agarose, fixing the protein expressed on a filter, reacting the purified and labeled Nectin-4 polypeptide with the above filter, and detecting the plaques expressing proteins bound to the Nectin-4 polypeptide according to the label. The polypeptide of the invention may be labeled by utilizing the binding between biotin and avidin, or by utilizing an antibody that specifically binds to the Nectin-4 polypeptide, or a peptide or polypeptide (for example, GST) that is fused to the Nectin-4 polypeptide. Methods using radioisotope or fluorescence and such may be also used.

Alternatively, in another embodiment of the screening method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet. 10: 286-92 (1994)”).

In the two-hybrid system, the polypeptide of the invention is fused to the SRF-binding region or GAL4-binding region and expressed in yeast cells. A cDNA library is prepared from cells expected to express a protein binding to the polypeptide of the invention, such that the library, when expressed, is fused to the VP16 or GAL4 transcriptional activation region. The cDNA library is then introduced into the above yeast cells and the cDNA derived from the library is isolated from the positive clones detected (when a protein binding to the polypeptide of the invention is expressed in yeast cells, the binding of the two activates a reporter gene, making positive clones detectable). A protein encoded by the cDNA can be prepared by introducing the cDNA isolated above to E. coli and expressing the protein. As a reporter gene, for example, Ade2 gene, lacZ gene, CAT gene, luciferase gene and such can be used in addition to the HIS3 gene.

A compound binding to the polypeptide encoded by Nectin-4 gene can also be screened using affinity chromatography. For example, the polypeptide of the invention may be immobilized on a carrier of an affinity column, and a test compound, containing a protein capable of binding to the polypeptide of the invention, is applied to the column. A test compound herein may be, for example, cell extracts, cell lysates, etc. After loading the test compound, the column is washed, and compounds bound to the polypeptide of the invention can be prepared. When the test compound is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon may be used as a mean for detecting or quantifying the bound compound in the present invention. When such a biosensor is used, the interaction between the polypeptide of the invention and a test compound can be observed real-time as a surface plasmon resonance signal, using only a minute amount of polypeptide and without labeling (for example, BIAcore, Pharmacia). Therefore, it is possible to evaluate the binding between the polypeptide of the invention and a test compound using a biosensor such as BIAcore.

The methods of screening for molecules that bind when the immobilized Nectin-4 polypeptide is exposed to synthetic chemical compounds, or natural substance banks or a random phage peptide display library, and the methods of screening using high-throughput based on combinatorial chemistry techniques (Wrighton et al., Science 273: 458-64 (1996); Verdine, Nature 384: 11-13 (1996); Hogan, Nature 384: 17-9 (1996)) to isolate not only proteins but chemical compounds that bind to the Nectin-4 protein (including agonist and antagonist) are well known to one skilled in the art.

Screening for a Compound Suppressing the Biological Activity of Nectin-4:

In the present invention the Nectin-4 protein has the activity of promoting cell proliferation of lung cancer cells (FIG. 4A), cell invasion activity (FIG. 4B), extracellular secretion (FIG. 1D), and Rac1 activation (FIG. 4D). Using these biological activities, the present invention provides a method for screening a compound that suppresses the proliferation of lung cancer cells, and a method for screening a compound for treating or preventing lung cancer. Thus, the present invention provides a method including the steps as follows:

(a) contacting a test compound with a polypeptide encoded by a polynucleotide of Nectin-4;

(b) detecting the biological activity of the polypeptide of step (a); and

(c) selecting the test compound that suppresses the biological activity as compared to the biological activity in the absence of the test compound.

According to the present invention, the therapeutic effect of the test compound on suppressing the activity to promote cell proliferation, or a candidate compound for treating or preventing cancer relating to Nectin-4 (e.g., lung bladder or cervical cancers) may be evaluated. Therefore, the present invention also provides a method of screening for a candidate compound for suppressing the cell proliferation, or a candidate compound for treating or preventing cancer relating to Nectin-4, using the Nectin-4 polypeptide or fragments thereof including the steps as follows:

(a) contacting a test compound with the Nectin-4 polypeptide or a functional fragment thereof;

(b) detecting the biological activity of the polypeptide or fragment of step (a), and

(c) correlating the biological activity of b) with the therapeutic effect of the test agent or compound.

In the context of present invention, the therapeutic effect may be correlated with the biological activity of a Nectin-4 polypeptide or a functional fragment thereof. For example, when the test agent or compound suppresses or inhibits the biological activity of a Nectin-4 polypeptide or a functional fragment thereof as compared to a level detected in the absence of the test agent or compound, the test agent or compound may identified or selected as the candidate agent or compound having the therapeutic effect. Alternatively, when the test agent or compound does not suppress or inhibit the biological activity of a Nectin-4 polypeptide or a functional fragment thereof as compared to a level detected in the absence of the test agent or compound, the test agent or compound may identified as the agent or compound having no significant therapeutic effect.

The method of the present invention will be described in more detail below.

Any polypeptides can be used for screening so long as they include the biological activity of the Nectin-4 protein. Such biological activity includes cell-proliferating activity, cell invasion activity, extracellular secretion, and Rac1 activation. For example, Nectin-4 protein can be used and polypeptides functionally equivalent to these proteins can also be used. Such polypeptides may be expressed endogenously or exogenously by cells.

In another aspect, the present invention also provides a screening method following the method described in Screening for a Nectin-4 binding compound, comprising the steps of:

a) contacting a test agent with the Nectin-4 polypeptide or a fragment thereof;

b) detecting the binding between the polypeptide or fragment and the test agent;

c) selecting the test agent that binds to the polypeptide;

d) contacting the test agent selected in step c) with the Nectin-4 polypeptide or a fragment thereof;

e) comparing the biological activity of the polypeptide or fragment with the biological activity detected in the absence of the agent; and

f) selecting the agent that suppresses the biological activity of the polypeptide as a candidate agent for treating or preventing lung cancer.

The compound isolated by this screening is a candidate for antagonists of the polypeptide encoded by Nectin-4 gene. The term “antagonist” refers to molecules that inhibit the function of the polypeptide by binding thereto. Said term also refers to molecules that reduce or inhibit expression of the gene encoding Nectin-4. Moreover, a compound isolated by this screening is a candidate for compounds which inhibit the in vivo interaction of the Nectin-4 polypeptide with molecules (including DNAs and proteins).

When the biological activity to be detected in the present method is cell proliferation, it can be detected, for example, by preparing cells which express the Nectin-4 polypeptide, culturing the cells in the presence of a test compound, and determining the speed of cell proliferation, measuring the cell cycle and such, as well as by measuring the colony forming activity, for example, shown in FIG. 4A. The compounds that reduce the speed of proliferation of cells expressed the Nectin-4 polypeptide compared with that of no compound treated cells and keep the speed of that compared with no or little those polypeptides expressed cells are selected as candidate compound for treating or preventing lung cancer.

When the biological activity to be detected in the present method is cell invasion activity, it can be detected, for example, by preparing cells which express Nectin-4 polypeptide and determining the amount of invention cells, measuring with matrigel invasion assay, for example, shown in FIG. 4B. The compounds that reduce the amount of invasion cells expressed Nectin-4 polypeptide compared with that of no compound treated cells and keep the amount of that compared with no or little Nectin-4 polypeptides expressed cells are selected as candidate compound for treating or preventing lung cancer.

When the biological activity to be detected in the present method is extracellular secretion, it can be detected, for example, by preparing cells which express Nectin-4 polypeptide, culturing the cells in the presence of a test compound, and determining the amount of secreted protein of those polypeptides in culture medium, measuring with ELISA, for example, shown in FIG. 1D. The compounds that reduce the amount of secreted protein from the cells expressed Nectin-4 polypeptide compared with that of no compound treated cells or Nectin-4 and keep the amount of that compared with no or little Nectin-4 polypeptides expressed cells are selected as candidate compound for treating or preventing lung cancer.

When the biological activity to be detected in the present method is Rac1 activation, it can be detected, for example, by preparing cells which express Nectin-4 polypeptide or cell lysate and determining the level of Rac1 activation, measuring with western blot, for example, shown in FIG. 4D. The term of “Rac1 activation” means increase of GTP-bound form of Rac1. The compounds that reduce the level of Rac1 activation in cells expressed Nectin-4 polypeptide compared with that of no compound treated cells and keep the amount of that compared with no or little Nectin-4 polypeptides expressed cells are selected as candidate compound for treating or preventing lung cancer.

For example, it was confirmed that Rac1 was co-expressed with Nectin-4 in lung cancer cells, and is likely to be a physiological substrate of guanylate cyclase suggesting that Nectin-4 could have a cell migration function in lung cancer cells through activation of Rac1 (FIG. 4D). Accordingly, compounds that inhibit the activation of Rac1 through the inhibition of Nectin-4 function is expected to suppress the proliferation of lung cancer cells, and thus is useful for treating or preventing lung cancer, especially NSCLC. Therefore, the present invention also provides a method for screening a compound that suppresses the proliferation of lung cancer cells, and a method for screening a compound for treating or preventing lung cancer, especially NSCLC.

More specifically, the method includes the steps of:

(a) contacting a candidate compound with cell lysate or cells which overexpress Nectin-4;

(b) measuring an activation form of Rac1; and

(c) selecting a candidate compound that reduces the activation form of Rac1 as compared to a control.

Preferably, the activation of Rac1 can also be evaluated by immunological technique using antibody recognizing activation of Rac1. For example, antibody recognizing GTP-bound form of Rac1 can be used for such purpose. In preferred embodiments, control level to be compared may be activation level of Rac1 detected in absence of the candidate compound under the condition same as test condition (in presence of the candidate compound).

In the present invention, methods for preparing polypeptides functionally equivalent to a given protein are well known by a person skilled in the art and include known methods of introducing mutations into the protein. Generally, it is known that modifications of one or more amino acid in a protein do not influence the function of the protein (Mark D F et al., Proc Natl Acad Sci USA 1984, 81: 5662-6; Zoller M J & Smith M, Nucleic Acids Res 1982, 10: 6487-500; Wang A et al., Science 1984, 224:1431-3; Dalbadie-McFarland G et al., Proc Natl Acad Sci USA 1982, 79: 6409-13). In fact, mutated or modified proteins, proteins having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence which alter a single amino acid or a small percentage of amino acids, or those considered to be “conservative modifications”, wherein the alteration of a protein results in a protein with similar functions, are contemplated in the context of the instant invention.

“Suppress the biological activity” as defined herein are preferably at least 10% suppression of the biological activity of Nectin-4 in comparison with in absence of the compound, more preferably at least 25%, 50% or 75% suppression and most preferably at 90% suppression.

Screening for a Compound Altering the Expression of Nectin-4:

In the present invention, the decrease of the expression of Nectin-4 by siRNA causes inhibiting cancer cell proliferation (FIG. 4A). Therefore, the present invention provides a method of screening for a compound that inhibits the expression of Nectin-4. A compound that inhibits the expression of Nectin-4 is expected to suppress the proliferation of lung cancer cells, and thus is useful for treating or preventing lung cancer. Therefore, the present invention also provides a method for screening a compound that suppresses the proliferation of lung cancer cells, and a method for screening a compound for treating or preventing lung cancer. In the context of the present invention, such screening may include, for example, the following steps:

(a) contacting a candidate compound with a cell expressing Nectin-4; and

(b) selecting the candidate compound that reduces the expression level of Nectin-4 as compared to a control.

According to the present invention, the therapeutic effect of the test agent or compound on inhibiting the cell growth or a candidate agent or compound for treating or preventing Nectin-4 associating disease may be evaluated. Therefore, the present invention also provides a method for screening a candidate agent or compound that suppresses the proliferation of cancer cells, and a method for screening a candidate agent or compound for treating or preventing Nectin-4 associating disease.

In the context of the present invention, such screening may include, for example, the following steps:

a) contacting a test agent or compound with a cell expressing the Nectin-4 gene;

b) detecting the expression level of the Nectin-4 gene; and

c) correlating the expression level of b) with the therapeutic effect of the test agent or compound.

In the present invention, the therapeutic effect may be correlated with the expression level of the Nectin-4 gene. For example, when the test agent or compound reduces the expression level of the Nectin-4 gene as compared to a level detected in the absence of the test agent or compound, the test agent or compound may identified or selected as the candidate agent or compound having the therapeutic effect. Alternatively, when the test agent or compound does not reduce the expression level of the Nectin-4 gene as compared to a level detected in the absence of the test agent or compound, the test agent or compound may identified as the agent or compound having no significant therapeutic effect.

The method of the present invention will be described in more detail below.

Cells expressing the Nectin-4 include, for example, cell lines established from lung cancer; such cells can be used for the above screening of the present invention (e.g., PC9, NCI-H1666, NCI-H358, and NCI-H2170). The expression level can be estimated by methods well known to one skilled in the art, for example, RT-PCR, Northern bolt assay, Western bolt assay, immunostaining and flow cytometry analysis. “Reduce the expression level” as defined herein are preferably at least 10% reduction of expression level of Nectin-4 in comparison to the expression level in absence of the compound, more preferably at least 25%, 50% or 75% reduced level and most preferably at 95% reduced level. The compound herein includes chemical compound, double-strand nucleotide, and so on. The preparation of the double-strand nucleotide is in aforementioned description. In the method of screening, a compound that reduces the expression level of Nectin-4 can be selected as candidate compounds to be used for the treatment or prevention of lung cancer.

Alternatively, the screening method of the present invention may include the following steps:

(a) contacting a candidate compound with a cell into which a vector, including the transcriptional regulatory region of Nectin-4 and a reporter gene that is expressed under the control of the transcriptional regulatory region, has been introduced;

(b) measuring the expression or activity of said reporter gene; and

(c) selecting the candidate compound that reduces the expression or activity of said reporter gene.

According to the present invention, the therapeutic effect of the test agent or compound on inhibiting the cell growth or a candidate agent or compound for treating or preventing Nectin-4 associating disease may be evaluated. Therefore, the present invention also provides a method for screening a candidate agent or compound that suppresses the proliferation of cancer cells, and a method for screening a candidate agent or compound for treating or preventing a Nectin-4 associated disease.

In the context of the present invention, such screening may include, for example, the following steps:

a) contacting a test agent or compound with a cell into which a vector, composed of the transcriptional regulatory region of the Nectin-4 gene and a reporter gene that is expressed under the control of the transcriptional regulatory region, has been introduced;

b) detecting the expression or activity of said reporter gene; and

c) correlating the expression level of b) with the therapeutic effect of the test agent or compound.

In the context of the present invention, the therapeutic effect may be correlated with the expression or activity of said reporter gene. For example, when the test agent or compound reduces the expression or activity of said reporter gene as compared to a level detected in the absence of the test agent or compound, the test agent or compound may identified or selected as the candidate agent or compound having the therapeutic effect. Alternatively, when the test agent or compound does not reduce the expression or activity of said reporter gene as compared to a level detected in the absence of the test agent or compound, the test agent or compound may identified as the agent or compound having no significant therapeutic effect.

Suitable reporter genes and host cells are well known in the art. For example, reporter genes are luciferase, green florescence protein (GFP), Discosoma sp. Red Fluorescent Protein (DsRed), Chrolamphenicol Acetyltransferase (CAT), lacZ and beta-glucuronidase (GUS), and host cell is COS7, HEK293, HeLa and so on. The reporter construct required for the screening can be prepared by connecting reporter gene sequence to the transcriptional regulatory region of Nectin-4. The transcriptional regulatory region of Nectin-4 herein is the region from start codon to at least 500 bp upstream, preferably 1000 bp, more preferably 5000 or 10000 bp upstream. A nucleotide segment containing the transcriptional regulatory region can be isolated from a genome library or can be propagated by PCR. The reporter construct required for the screening can be prepared by connecting reporter gene sequence to the transcriptional regulatory region of any one of these genes. Methods for identifying a transcriptional regulatory region, and also assay protocol are well known (Molecular Cloning third edition chapter 17, 2001, Cold Springs Harbor Laboratory Press).

The vector containing the said reporter construct is infected to host cells and the expression or activity of the reporter gene is detected by method well known in the art (e.g., using luminometer, absorption spectrometer, flow cytometer and so on). “reduces the expression or activity” as defined herein are preferably at least 10% reduction of the expression or activity of the reporter gene in comparison with in absence of the compound, more preferably at least 25%, 50% or 75% reduction and most preferably at 95% reduction.

In the context of the present invention, candidate compounds that have the potential to treat or prevent cancers can be identified. The therapeutic potential of these candidate compounds may be evaluated by second and/or further screening to identify therapeutic agent for cancers. For example, when a compound binding to Nectin-4 protein inhibits described above activities of the cancer, it may be concluded that such compound has the Nectin-4 specific therapeutic effect.

Aspects of the present invention are described in the following examples, which are not intended to limit the scope of the invention described in the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

The invention will be further described in the following examples, which do not limit the scope of the invention described in the claims.

### EXAMPLES

The invention will be further described in the following examples, which do not limit the scope of the invention described in the claims.

### Example 1

### General Methods

Cell lines and clinical tissue samples.

Twenty human lung-cancer cell lines used in this study are as follows: A549, LC319, H1781, PC-3, PC-9, PC14, A427, NCI-H1666, NCI-H358, RERF-LC-AI, SK-MES-1, EBC-1, LU61, NCI-H520, NCI-H1703, NCI-H2170, NCI-H226, NCI-H647, LX1, and SBC-5. Human bronchial epithelial cells, BEAS-2B was included in the panel of the cells used in this study. All cells were grown in monolayers in appropriate media supplemented with 10% fetal calf serum (FCS) and were maintained at 37 degrees C. in an atmosphere of humidified air with 5% CO₂. Primary lung cancer samples as well as their corresponding normal tissues adjacent to resection margins from patients, who had no anticancer treatment before tumor resection, had been obtained earlier with informed consent (Kikuchi T, et al. Oncogene 2003; 22:2192-205). The histological classification of the tumor specimens was based on WHO criteria (Travis W D, et al. World Health Organization International Histological Classification of Tumors, 3rd edn. Berlin: Springer, 1999). All tumors were staged on the basis of the pTNM pathological classification of the UICC (International Union Against Cancer; Table 2 (Sobin L, Wittekind Ch. TNM Classification of Malignant Tumours, 6th edition. New York: Wiley-Liss; 2002). A total of 422 formalin-fixed samples of primary NSCLCs including 265 adenocarcinomas (ADCs), 116 squamous-cell carcinomas (SCCs), 28 large cell carcinomas (LCCs), 13 adenosquamous-cell carcinomas (ASCs) and adjacent normal lung tissue, had been obtained earlier along with clinicopathological data from patients who had undergone surgery at Saitama Cancer Center (Saitama, Japan). This study and the use of all clinical materials were approved by the individual Institutional Research Ethics Committees.

Serum Samples.

Serum samples were obtained with informed consent from 131 healthy volunteers as controls (88 males and 43 females; median age (+/−1SD) 55.5+/−9.6, range 31-83; Table 6) and from 86 non-neoplastic lung disease patients with chronic obstructive pulmonary disease (COPD) enrolled as a part of the Japanese Project for Personalized Medicine (BioBank Japan) or admitted to Hiroshima University and its affiliated Hospitals (76 males and 10 females; median age (+/−1SD) 66.4+/−5.6, range 54-73; Table 7). All of these COPD patients were current and/or former smokers (The mean [+/−1SD] of pack-year index (PYI) was 66.7+/−44.4; PYI was defined as the number of cigarette packs [20 cigarette per pack] consumed a day multiplied by years). Serum samples were obtained with informed consent from 164 NSCLC patients (123 ADCs and 41 SCCs) admitted to Hiroshima University Hospital, as well as Kanagawa Cancer Center Hospital (122 males and 42 females; median age (+/−1SD) 64.5+/−10.4, range 30-85; Table 4). To investigate the prognostic value of serum Nectin-4, two additional sets of serum samples with precise follow-up record after treatments were also obtained; Group-1: serum samples obtained with informed consent before curative surgical resection at Kanagawa Cancer Center Hospital from 95 early stage NSCLC patients (stage I) (48 males and 47 females; median age (+/−1SD) 66.8+/−9.6, range 38-84; Group-2: serum samples obtained at diagnosis with informed consent from 62 advanced stage NSCLC patients (stage IIIB-IV) treated later with an identical protocol of the 1st-line chemotherapy using both carboplatin and paclitaxel admitted to Hiroshima University Hospital (46 males and 16 females; median age (+/−1SD) 61.7+/−10.2, range 35-79). Patient samples were selected for the study on the basis of the following criteria: (1) patients were newly diagnosed and previously untreated and (2) their tumors were pathologically diagnosed as NSCLCs (stages I-IV). Serum was obtained at the time of diagnosis and stored at −150 degrees C.

Semi-Quantitative RT-PCR Analysis.

Total RNA was extracted from cultured cells and clinical tissues using Trizol reagent (Life Technologies) according to the manufacturer's protocol. Extracted RNAs and normal human-tissue polyA RNAs were treated with DNase I (Roche Diagnostics) and then reverse-transcribed using oligo (dT)₁₂₋₁₈ primer and SuperScript II reverse transcriptase (Life Technologies, Inc.). Semi-quantitative RT-PCR experiments were carried out with synthesized Nectin-4 gene-specific primers (5′-GTCAGCCAGAGGCTTGAACT-3′ (SEQ ID NO: 3) and 5′-GGATTCAAAGCAGGCACAGT-3′ (SEQ ID NO: 4)), or with beta-actin (ACTB)-specific primers (5′-ATCAAGATCATTGCTCCTCCT-3′ (SEQ ID NO: 5) and 5′-CTGCGCAAGTTAGGTTTTGT-3′ (SEQ ID NO: 6)) as an internal control. All PCR reactions involved initial denaturation at 94 degrees C. for 2 min followed by 22 (for ACTB) or 35 cycles (for Nectin-4) of 94 degrees C. for 30 s, 54 or 60 degrees C. for 30 s, and 72 degrees C. for 60 s on a GeneAmp PCR system 9700 (Applied Biosystems). Appropriate dilutions of each single-stranded cDNA prepared from mRNAs of clinical lung cancer samples were prepared by taking the level of beta-actin (ACTB) expression as a quantitative control.

Northern-Blot Analysis.

Human multiple-tissue blots (BD Biosciences Clontech) were hybridized with a ³²P-labeled PCR product of Nectin-4. PCR product of Nectin-4 that was prepared as a probe by reverse transcription-PCR (RT-PCR) using primers 5′-CAGGGGGTTAATTCCTGTGA-3′ (SEQ ID NO: 7) and 5′-CAACTAATGGAAAGGGCAAGA-3′ (SEQ ID NO: 8). Prehybridization, hybridization, and washing were performed according to the supplier's recommendations. The blots were autoradiographed with intensifying screens at −80 degrees C. for 7 days.

Generation of Murine Anti-Nectin-4 Monoclonal Antibody.

The anti-Nectin-4 monoclonal antibodies (mAbs) were produced using standard method after successive intraperitoneal injections of 20 microgram soluble recombinant protein (Nectin-4 ectodomain) to mice. Two monoclonal antibodies (clones 19-33 and 66-97) to Nectin-4 were proven to be specific for endogenous human Nectin-4, by immunocytochemistry, flow cytometry, and ELISA for the culture media using lung-cancer cell lines with high levels of endogenous Nectin-4 expression or those without Nectin-4 expression as well as by immunoprecipitation using COS-7 cells transfected with Nectin-4 expression vector. Exogenously expressed myc-tagged Nectin-4 protein in COS-7 cells was immunoprecipitated by either of two monoclonal antibodies to Nectin-4 (clones 19-33 and 66-97) and immunoblotted with anti-myc antibodies. Theses monoclonal antibodies (clones 19-33 and 66-97) could specifically immunoprecipitate Nectin-4 protein in myc-tagged Nectin-4 overexpressing cells, but not in mock-transformant cells, suggesting that these antibodies have an ability to specifically recognize the native Nectin-4.

Flow Cytometry.

2×10⁵ cells were incubated for 30 min at 4 degrees C. with 2.5 microgram/ml of mouse anti-Nectin-4 mAb (generated to recombinant Nectin-4; please see above) or control mouse IgG (Beckman Coulter), washed, and then revealed by incubation for 30 min at 4 degrees C. with AlexaFluor 488-conjugated anti-mouse IgG (Molecular Probes). The cells were washed in PBS and analyzed on a FACScan flow cytometer (Becton Dickinson Labware) and analyzed by ModFit software (Verity Software House, Inc.).

Immunocytochemical Analysis.

Cultured cells were washed twice with PBS(−), fixed in 4% paraformaldehyde solution for 10 min at room temperature, and then rendered permeable with PBS(−) containing 0.1% Triton X-100 for two min. Prior to the primary antibody reaction, cells were covered with CAS Block for seven min to block nonspecific antibody binding. After the cells were incubated with 10 microgram/ml of mouse anti-Nectin-4 mAb (generated to recombinant Nectin-4) for detecting endogenous Nectin-4 or 2 microgram/ml of mouse anti-myc mAb (9E10; Santa Cruz) for myc-tagged Nectin-4, Alexa 488-conjugated goat anti-mouse secondary antibody (Molecular Probe) was added to reveal Nectin-4. Nuclei were stained with 4′,6-diamidino-2-phenylindole (DAPI). To visualize F-actin filaments, Alexa594-conjugated phalloidin (Molecular Probes) was added after the incubation with secondary antibodies. The stained cells were viewed with a laser-confocal microscope (TSC SP2 AOBS: Leica Microsystems).

Immunohistochemistry and Tissue Microarray.

Tumor-tissue microarrays were constructed using 422 formalin-fixed primary NSCLCs as described elsewhere (Chin S F, et al. Mol Pathol 2003; 56: 275-9, Callagy G, et al. Diagn Mol Pathol 2003; 12: 27-34, Callagy G, et al. J Pathol 2005; 205:388-96). Briefly, the tissue area for sampling was selected based on visual alignment with the corresponding HE-stained section on a slide. Three, four, or five tissue cores (diameter 0.6 mm; height 3-4 mm) taken from a donor tumor block were placed into a recipient paraffin block using a tissue microarrayer (Beecher Instruments). A core of normal tissue was punched from each case, and 5-micrometer sections of the resulting microarray block were used for immunohistochemical analysis.

To investigate the status of the Nectin-4 protein in clinical lung-cancer samples that had been embedded in paraffin blocks, the sections were stained in the following manner. Briefly, 16.25 microgram/ml of mouse anti-human Nectin-4 antibody (generated to recombinant Nectin-4) was added after blocking of endogenous peroxidase and proteins. The sections were incubated with HRP-labeled anti-mouse IgG as the secondary antibody. Substrate-chromogen was added and the specimens were counterstained with hematoxylin. Since the intensity of staining within each tumor tissue core was mostly homogenous, the intensity of Nectin-4 staining was semi-quantitatively evaluated by three independent investigators without prior knowledge of clinicopathological data using following criteria: strong positive (scored as 2+), dark brown staining in more than 50% of tumor cells completely obscuring plasma membrane and cytoplasm; weak positive (1+), any lesser degree of brown staining appreciable in tumor cell plasma membrane and cytoplasm; absent (scored as 0), no appreciable staining in tumor cells. Cases were defined as strongly positive if all of the three reviewers independently classified them as such.

Statistical Analysis.

Contingency tables were used to analyze the relationship of Nectin-4 expression levels in tissues or serum and clinicopathological variables of NSCLC patients. Survival curves were calculated from the date of surgery or diagnosis to the time of death, or to the last follow-up observation. Kaplan-Meier curves were calculated for each relevant variable, and for Nectin-4 expression in lung tumors or for serum Nectin-4 levels; differences in survival times among patient subgroups were analyzed using the log-rank test. Univariate and multivariate analyses were performed with the Cox proportional-hazard regression model to determine associations between clinicopathological variables and cancer-related mortality. First, associations between death and possible prognostic factors including age, gender, histological type, pT-classification, and pN-classification, taking into consideration one factor at a time, were analyzed. Second, multivariate Cox analysis was applied on backward (stepwise) procedures that always forced Nectin-4 expression into the model, along with any and all variables that satisfied an entry level of a P value smaller than 0.05. As the model continued to add factors, independent factors did not exceed an exit level of P<0.05.

In ELISA, differences in the serum levels of Nectin-4, CEA, and CYFRA21-1 between tumor groups and a healthy control were analyzed by Mann-Whitney U tests.

ELISA.

Serum levels of Nectin-4 were measured by sandwich-type ELISA that was originally developed using mouse anti-human Nectin-4 antibodies (generated to recombinant Nectin-4; please see above). In brief, for detection of Nectin-4 in serum, 96-well flexible microtiter plates (439454; NALGE NUNC International) were coated with 5 microgram/ml of capturing monoclonal antibody to Nectin-4 (clone 19-33) overnight. Wells were blocked with 300 microliter PBS (pH 7.4) containing 1% BSA for two hours and then incubated for 2 hours with 3-fold diluted serum samples in PBS (pH 7.4) containing 1% BSA. After washing with PBS (pH 7.4) containing 0.05% Tween 20, the wells were incubated for two hours with 10 ng/ml of biotin-conjugated monoclonal anti-Nectin-4 antibody (clone 66-97), followed by reaction with avidin-conjugated peroxidase (P347; Dako Cytomation) using a Substrate Reagent (R&D Systems). The color reaction was stopped by addition of 50 microliter 2N sulfuric acid. Color intensity was determined by a photometer at a wavelength of 450 nm, with a reference wave-length of 570 nm. Standard curve was drawn for each plate using recombinant soluble Nectin-4 proteins (Nectin-4 ectodomain). Serum levels of Nectin-4 were calculated using dilutions of the recombinant Nectin-4 protein (ranging from 0.1 ng/ml to 300 ng/ml) as a reference. Levels of carcinoembryonic antigen (CEA) in serum were measured by ELISA with a commercially available enzyme test kit (Hope Laboratories) according to the supplier's recommendations. Levels of cytokeratin 19-fragment (CYFRA 21-1) in serum were measured by ELISA with a commercially available kit (DRG). Differences in the levels of Nectin-4, CEA, and CYFRA 21-1 between tumor groups and a healthy control group were analyzed by Mann-Whitney U tests. The levels of Nectin-4, CEA, and CYFRA 21-1 were further evaluated by receiver-operating characteristic (ROC) curve analysis to determine cut-off levels with optimal diagnostic accuracy and likelihood ratios. The correlation coefficients between Nectin-4 and CEA/CYFRA 21-1 as well as between CEA and CYFRA 21-1 were calculated with Spearman rank correlation. Significance was defined as P<0.05.

RNA Interference Assay.

To evaluate the biological functions of Nectin-4 in lung cancer cells, small interfering RNA (siRNA) duplexes against the target genes (Dharmacon) were used. The target sequences of the synthetic oligonucleotides for RNAi were as follows: control 1 (a nonspecific control oligo [CNT]; Dharmacon catalog no. D-001810-01-05); control 2 (Luciferase [LUC]: Photinus pyralis luciferase gene), 5′-CGUACGCGGAAUACUUCGA-3′ (SEQ ID NO: 9); siRNA-Nectin-4-#1, 5′-ACAGUUACCACGUCUGAGGUU-3′ (SEQ ID NO: 10 as target sequence), siRNA-Nectin-4-#2, 5′-AAUGGUUCAUGGCCUGUUUUU-3′ (SEQ ID NO: 11 as target sequence).

Lung cancer cell lines NCI-H2170 and NCI-H358 were plated onto 10-cm dishes (1×10⁶ cells per dish), and transfected with either of the siRNA oligonucleotides (100 nM), using 30 microliter of Lipofectamine 2000 (Invitrogen), according to the manufacturers' instructions. After 5 days of incubation, these cells were stained by Giemsa solution to assess colony formation, and cell numbers were assessed by 3-(4,5-dimethylthiazol-2-yl)-2,5-diphenyltetrazolium bromide (MTT) assay using cell-counting kit-8 solution (DOJINDO LABORATORIES).

Establishment of PC-14 Cells Stably Expressing Nectin-4

To establish PC-14 cells stably expressing Nectin-4, Myc/His-tagged Nectin-4 expression vector (pcDNA3.1 myc/His-Nectin-4) or mock vector (pcDNA3.1-myc/His) was transfected into PC-14 cells that did not express endogenous Nectin-4 using FuGENE6 transfection reagent (Roche). Transfected cells were incubated in the culture medium containing 0.6 mg/mL neomycin (Geneticin, Invitrogen) for 14 days. Then, 50 colonies were trypsinized and screened for stable transfectants by a limiting-dilution assay. Expression of Nectin-4 was determined in each clone by RT-PCR, Western blotting and immunocytochemical staining. Cell viability of two stable clones (PC-14-Nectin-4-#A and -#B) and two control clones (PC14-Mock-#A and -#B) was quantified with MTT assay in 7 days. All assays were performed in triplicate wells three independent times.

### Tumor Cell Grafting

To examine in vivo tumor growth promotion by Nectin-4 overexpression, above established PC-14 cells stably expressing Nectin-4 or those transfected with mock plasmids (3×10⁵ cells) were injected subcutaneously into the posterior dorsum of six BALB/cAJcl-nu/nu mice (female, 6 weeks old). Tumor volumes were measured and estimated for 20 days, by the following formula: V=0.5×(longer diameter)×(shorter diameter)₂. The animal experiments were conducted according to the institutional and national guidelines for the care and use of laboratory animals, and approved by the institutional animal use committee.

Matrigel Invasion Assay.

COS-7 and NIH-3T3 cells transfected either with pcDNA3.1-myc/His plasmids expressing human Nectin-4 or with mock plasmids were grown to near confluence in DMEM containing 10% FCS. The cells were harvested by trypsinization, washed in DMEM without addition of serum or proteinase inhibitor, and suspended in DMEM at concentration of 1×10⁵ cells/ml. Before preparing the cell suspension, the dried layer of Matrigel matrix (Becton Dickinson Labware) was rehydrated with DMEM for 2 hours at room temperature. DMEM (0.75 ml) containing 10% FCS was added to each lower chamber in 24-well Matrigel invasion chambers, and 0.5 ml (5×10⁴ cells) of cell suspension was added to each insert of the upper chamber. The plates of inserts were incubated for 22 hours at 37 degrees C. After incubation the chambers were processed; cells invading through the Matrigel were fixed and stained by Giemsa as directed by the supplier (Becton Dickinson Labware).

Rac1 Activation Assay.

Activation of Rac1 by Nectin-4 overexpression was detected according to the supplier's recommendations using Rac1 activation assay kit (Cell Biolabs). Firstly, COS-7 and NIH-3T3 cells transfected with Nectin-4-expressing pcDNA3.1-myc/His plasmids or mock plasmids were cultured for 48 hours, washed, and lysed with ice-cold 1× Assay Buffer. After centrifugation at 14,000×g for 10 min, each lysate was mixed with the p21-binding domain (PBD) of p21-activated protein kinase (PAK)-agarose beads at 4 degrees C. for 1 hour. The beads were washed three times with 1× Assay buffer. The pulled-down precipitant that contained PAK-PBD/GTP-Rac1 (activated Rac1) complex was washed and boiled with sample buffer to serve for western blot analysis using anti-Rac1 antibody.

### Example 2

### Nectin-4 Expression in Lung Tumors and Normal Tissues

To search for novel target molecules for the development of therapeutic agents and/or diagnostic biomarkers for NSCLC, genes were first screened, which showed more than a 5-fold higher level of expression in cancer cells than in normal cells, in the majority of NSCLCs analyzed by cDNA microarray. Among 27,648 genes and ESTs screened, Nectin-4 transcript was identified as a good candidate that showed more than a 5-fold higher level of expression in 87.5% of NSCLCs. Its transactivation was confirmed by semi-quantitative RT-PCR experiments in 9 of 10 additional NSCLC tissues and in 7 of 20 lung-cancer cell lines (FIG. 1A). Further, in another semi-quantitative RT-PCR experiments, its transactivation was confirmed in 11 of 14 additional NSCLC cases (6 of 7 adenocarcinomas (ADCs); 5 of 7 squamous cell carcinomas (SCCs)), and in 7 of 20 lung-cancer cell lines (FIG. 1E). Original mouse monoclonal antibody specific to human Nectin-4 was subsequently generated, to thereby obtain two independent clones, 19-33 and 66-97. As Nectin-4 was indicated to be a type I membrane protein, Nectin-4 expression on the surface of lung-cancer cells was attempted to validate using flow-cytometry with anti-Nectin-4 monoclonal antibody (clone 19-33). This analysis indicated that Nectin-4 protein was strongly expressed and localized at the plasma membranes of NCI-H2170 and NCI-H358 cells, in which Nectin-4 protein had been detected at a high level by semi-quantitative RT-PCR, but not in those of A549 and SBC-5 cells, which had not expressed endogenous Nectin-4 (FIG. 1B). Immunocytochemical staining of the same set of lung cancer cells with anti-Nectin-4 monoclonal antibody (clone 19-33) indicated that Nectin-4 was detected at plasma membrane and cytoplasm in NCI-H2170 and NCI-H358, but not in A549 and SBC-5 cells (FIG. 1C). Since the ectodomain of Nectin-4 (43.5 kDa) was suggested to be secreted by cleavage of its extracellular portion (Fabre-Lafay S, et al. J Biol Chem 2005; 280:19543-50), ELISA was applied to examine its presence in the culture media of the lung-cancer cell lines. The amounts of detectable Nectin-4 in the culture media was concordant to the expression levels of Nectin-4 detected with semi-quantitative RT-PCR, flow-cytometry and immunofluorescence analyses (FIG. 1D), further supporting the specific binding affinity of the monoclonal antibodies to Nectin-4. Attempts to measure endogenous Nectin-4 protein levels in the NCI-H2170 and NCI-H358 cell lines by western blotting using the two monoclonal antibodies (clones 19-33 and 66-97) failed to detect any bands on western-blots, indicating that these antibodies can be used for recognition of a native form of Nectin-4. It was further confirmed that the two monoclonal antibodies (19-33 and 66-97) could specifically immunoprecipitate Nectin-4 protein in myc-tagged Nectin-4-overexpressing COS-7 cells (FIG. 1F, left panel), but not in mock-transformant COS-7 cells (FIG. 1F, right panel), suggesting that these antibodies have an ability to specifically recognize the native Nectin-4. Northern blotting using Nectin-4 cDNA as a probe identified the 3.7-kb transcript as a very faint signal in placenta and to a lesser degree in trachea among the 23 normal human tissues examined (data not shown), which was concordant with previous report (Reymond N, et al. J Biol Chem 2001; 276:43205-15). Expression of Nectin-4 protein was subsequently examined in six normal tissues (heart, lung, liver, kidney, trachea and placenta), as well as lung cancers using anti-Nectin-4 monoclonal antibody, and it was found that the expression of Nectin-4 protein in the former five tissues was hardly detectable while positive Nectin-4 staining appeared in placenta and lung tumor tissues. The expression levels of Nectin-4 protein in lung cancer were significantly higher than those in placenta (FIG. 2A).

### Example 3

### Association of Nectin-4 Expression with Poor Clinical Outcomes for NSCLC Patients

To further verify the biological and clinicopathological significance of Nectin-4, the expression of Nectin-4 protein was examined by means of tissue microarrays consisting of NSCLC tissues from 422 patients who underwent surgical resection. A pattern of Nectin-4 expression was classified on the tissue array ranging from absent/weak (scored as 0˜1+) to strong (2+) (FIG. 2B, top panels). Of the 422 NSCLC cases examined, Nectin-4 was strongly stained in 245 (58.1%; score 2+), weakly stained in 119 (28.2%; score 1+), and not stained in 58 cases (13.7%; score 0), whereas their adjacent normal lung tissues were not stained (FIG. 2B, middle panels). Then, the association between Nectin-4 status and clinicopathological parameters was evaluated. As shown in Table 2, histological type (higher in ADC; P=0.0059 by Fisher's exact test) and pT factor (higher in T2-T4; P=0.0048 by Fisher's exact test) were significantly associated with the strong Nectin-4 positivity (score 2+).

TABLE 2
Association between Nectin4-positivity in NSCLC tissues
and patients' characteristics (n = 422)

| | | Nectin4 | Nectin4 | | |
|---|---|---|---|---|---|
| | | strong | weak | Nectin4 | P-value |
| | Total | positive | positive | absent | strong vs |
| | n = 422 | n = 245 | n = 119 | n = 58 | weak/absent |
| Gender | | | | | |
| Male | 290 | 172 | 76 | 42 | 0.4577 |
| Female | 132 | 73 | 43 | 16 | |
| Age (years) | | | | | |
| <65 | 218 | 126 | 63 | 29 | 0.9217 |
| >=65 | 204 | 119 | 56 | 29 | |
| Histological type | | | | | |
| ADC | 265 | 167 | 68 | 30 | 0.0059^{+,}** |
| SCC | 116 | 54 | 41 | 21 | |
| Others | 41 | 24 | 10 | 7 | |
| pT factor | | | | | |
| T1 | 143 | 69 | 51 | 23 | 0.0048** |
| T2 − T4 | 279 | 176 | 68 | 35 | |
| pN factor | | | | | |
| N0 | 259 | 142 | 81 | 36 | 0.105 |
| N1 + N2 | 163 | 103 | 38 | 22 | |
| Smoking | | | | | |
| non-smoker | 127 | 70 | 40 | 17 | 0.4523 |
| smoker | 295 | 175 | 79 | 41 | |

ADC, adenocarcinoma; SCC, squamous-cell carcinoma
Others, large-cell carcinoma (LCC) plus adenosquamous-cell carcinoma (ASC)
⁺ADC versus non-ADC
**P < 0.05 (Fisher's exact test)

NSCLC patients whose tumors showed strong Nectin-4 expression revealed shorter survival periods compared to those with absent/weak Nectin-4 expression (P<0.0001 by log-rank test; FIG. 2B, bottom panels). Univariate analysis was also applied to evaluate associations between patient prognosis and other factors including age (<65 versus 65≧), gender (female versus male), histological type (ADC versus non-ADC), pT classification (T1 versus T2-4), pN classification (N0 versus N1+N2), smoking history (non-smoker versus smoker) and Nectin-4 expression status (score 0+, 1+ versus 2+). Among those parameters, strong Nectin-4 positivity (P<0.0001), elderly (P=0.0079), male (P=0.0011), non-ADC(P=0.0208), advanced pT stage (P<0.0001), and advanced pN stage (P<0.0001) were significantly associated with poor prognosis. Multivariate analysis of these prognostic factors revealed that strong Nectin-4 expression, elderly, larger tumor size, and lymph node metastasis were independent prognostic factors for NSCLC patients (P<0.0001, 0.0006, 0.0006, <0.0001, respectively; Table 3).

TABLE 3
Cox's proportional hazards model analysis of prognostic factors in patients with NSCLCs

| | Hazards | | Unfavorable/ | |
|---|---|---|---|---|
| Variables | ratio | 95% Cl | Favorable | P-value |
| Univariate analysis | | | | |
| Nectin4 | 2.116 | 1.551-2.887 | Strong(2+)/ | <0.0001* |
| | | | Weak(1+) or (−) | |
| Age (years) | 1.473 | 1.107-1.962 | >=65/<65 | 0.0079* |
| Gender | 1.738 | 1.247-2.422 | Male/Female | 0.0011* |
| Histological | 1.402 | 1.053-1.867 | non-ADC/ADC | 0.0208* |
| type | | | | |
| pT factor | 2.66 | 1.857-3.810 | T2-T4/T1 | <0.0001* |
| pN factor | 2.393 | 1.799-3.181 | N1 + N2/N0 | <0.0001* |
| smoking | 1.248 | 0.907-1.716 | smoker/non-smoker | 0.1734 |
| Multivariate analysis | | | | |
| Nectin4 | 2.145 | 1.558-2.954 | Strong(2+)/ | <0.0001* |
| | | | Weak(1+) or (−) | |
| Age (years) | 1.679 | 1.250-2.254 | >=65/<65 | 0.0006* |
| Gender | 1.437 | 0.996-2.072 | Male/Female | 0.0522 |
| Histological | 1.144 | 0.828-1.580 | non-ADC/ADC | 0.4158 |
| type | | | | |
| pT factor | 1.916 | 1.319-2.785 | T2-T4/T1 | 0.0006* |
| pN factor | 2.324 | 1.728-3.124 | N1 + N2/N0 | <0.0001* |

ADC, adenocarcinoma,
*P < 0.05

### Example 4

### Serum Levels of Nectin-4 in NSCLC Patients

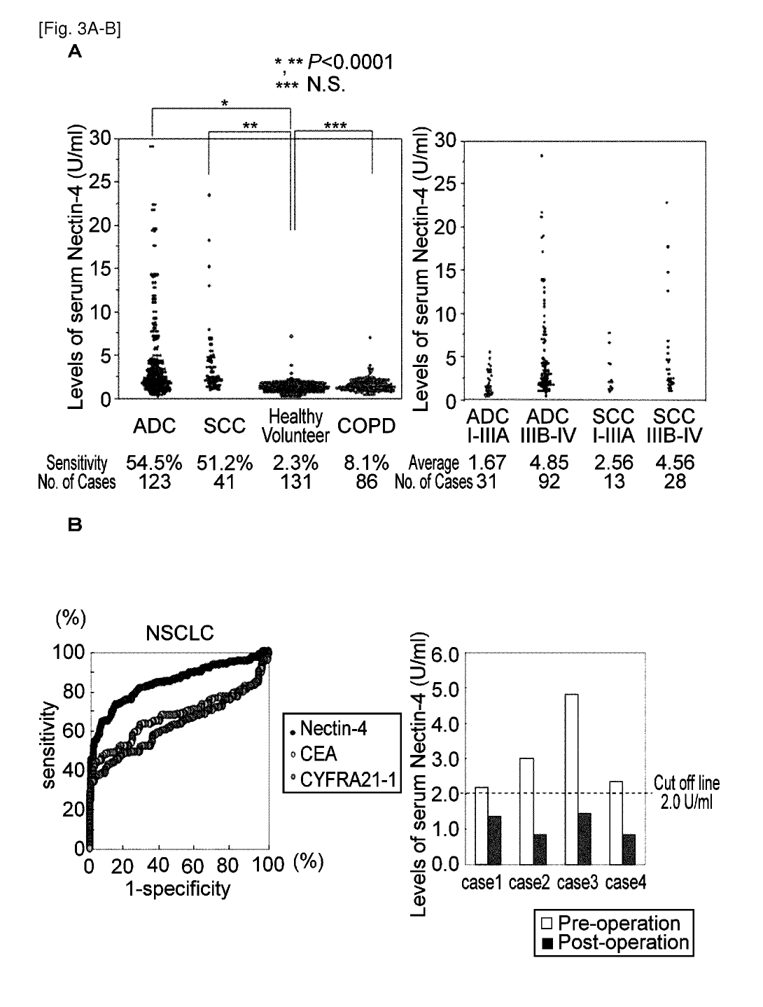

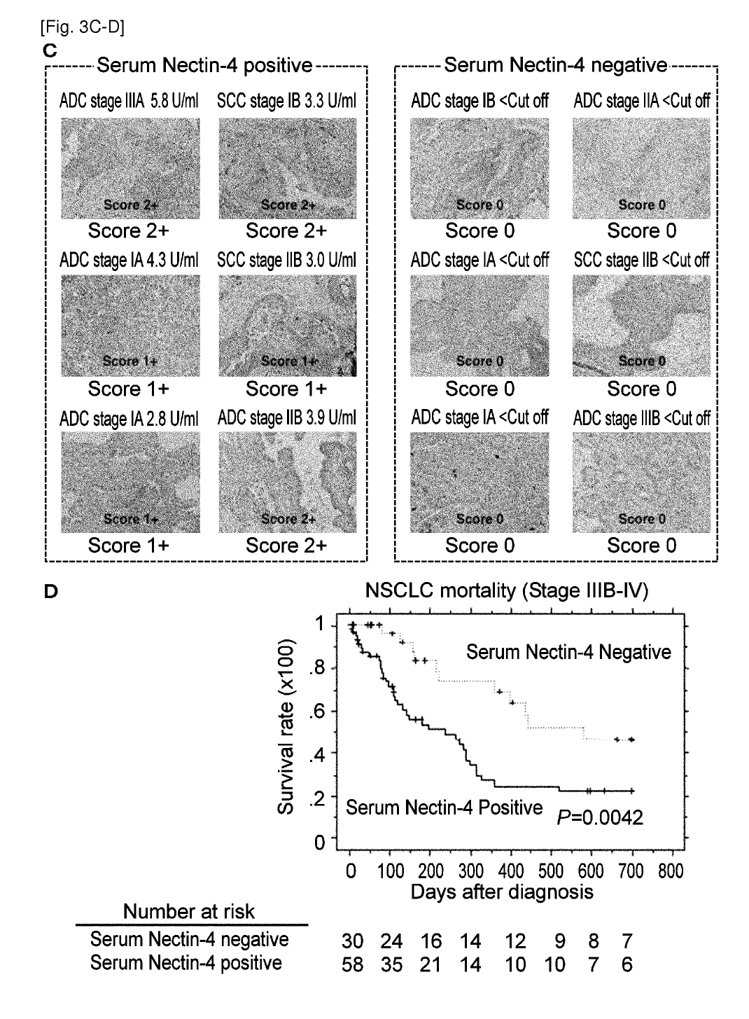

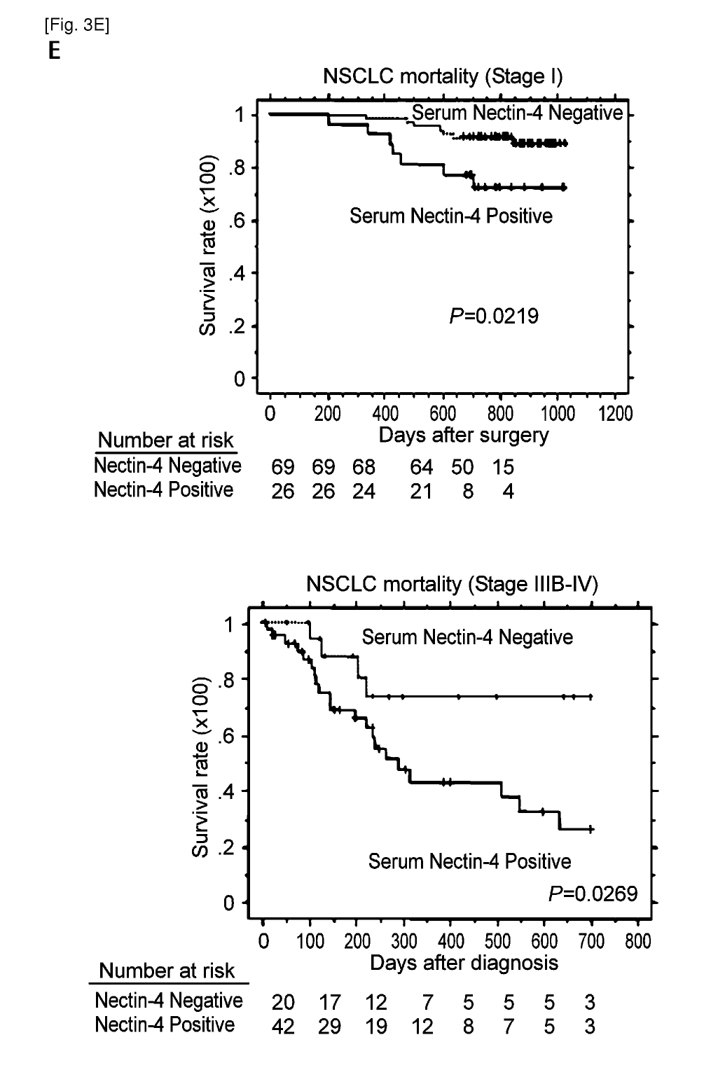

As the in vitro findings had suggested a possibility for the development of a serum lung cancer biomarker using Nectin-4 (FIG. 1D), it was investigated whether the Nectin-4 is secreted into sera of patients with NSCLC. ELISA experiments detected Nectin-4 in serological samples from the majority of the 164 patients with NSCLC (FIG. 3A); serum levels of Nectin-4 in NSCLC patients were 4.0+/−4.9 units/ml (2.0+/−2.5 ng/ml) (mean+/−1SD). In contrast, the mean (+/−1SD) serum levels of Nectin-4 in 131 healthy volunteers were 1.1+/−0.7 units/ml (0.6+/−0.4 ng/ml), and those in 86 patients with COPD, who were current and/or former smokers were 0.6+/−0.9 ng/ml. The difference in the level of serum Nectin-4 protein between NSCLC patients and healthy volunteers was significant with P-value of <0.0001 (Mann-Whitney U test). The difference between healthy volunteers and COPD patients was not significant (P=0.103 by Mann-Whitney U test). When classified according to histological type, the serum levels of Nectin-4 were 4.0+/−5.0 units/ml (2.0+/−2.5 ng/ml) in ADC patients and 3.9+/−4.6 units/ml (2.0+/−2.3 ng/ml) in SCC patients (FIG. 3A, left panel). High levels of serum Nectin-4 were detected even in patients with earlier-stage tumors (stages I-IIIA) (FIG. 3A, right panel). Using receiver-operating characteristic (ROC) curves drawn with the data of these 164 NSCLC patients and 131 healthy donors (FIG. 3B, left panel), the cut-off level in this assay was set to provide optimal diagnostic accuracy and likelihood ratios (minimal false negative and false positive results) for Nectin-4, i.e., 2.0 units/ml (1.0 ng/ml) with a sensitivity of 53.7% ( 88/164 NSCLC; 6 of 24 (25%) stage I, 10 of 20 (50%) stage II-IIIA, and 72 of 120 (60%) stage IIIB-IV tumors) and a specificity of 97.7% ( 128/131) (2.3% ( 3/131)) (Table 4).

TABLE 4
Sensitivity and specificity of serum Nectin-4,
CEA, and CYFR21-1 in detecting NSCLCs

| | | | lung | | normal |
|---|---|---|---|---|---|
| | | | cancer | | volun- |
| | | | patients | | teers |
| | total | | n-164 | | n = 131 |
| Nectin-4 | | | | | |
| surum Nectin-4 | 91 | | 88 | | 3 |
| positive | | | | | |
| surum Nectin-4 | 204 | | 76 | | 128 |
| negative | | | | | |
| | | sensitivity | 53.70% | specificity | 97.7% |
| CEA | | | | | |
| serum CEA | 73 | | 70 | | 3 |
| positive | | | | | |
| serum CEA | 222 | | 94 | | 128 |
| negative | | | | | |
| | | sensitivity | | specificity | |
| CYFRA21-1 | | | | | |
| serum CYFRA21-1 | 69 | | 64 | | 5 |
| positi | | | | | |
| serum CYFRA21-1 | 226 | | 100 | | 126 |
| negat | | | | | |
| | | sensitivity | 39.0% | specificity | 96.2% |

To evaluate the potential of serum Nectin-4 level as a cancer-specific biomarker, the relationship between serum Nectin-4 positivity and patients' or healthy individuals' characteristics was examined (Tables 5-7). Serum Nectin-4 positivity was significantly associated with pT factor (higher in T2-T4; P=0.0039 by Fisher's exact test) in NSCLC patients (Table 5). Importantly, serum Nectin-4 positivity was not associated with smoking history, gender, and age in individual groups of lung cancer, COPD, and healthy volunteer (Tables 5-7). In addition, there was no significant association between its positivity and respiratory function in COPD patients (Table 7). To evaluate the feasibility of using serum Nectin-4 level as a tumor-detection biomarker, serum levels of two conventional tumor markers (CEA and CYFRA 21-1) were also measured for NSCLC patients in the same set of serum samples from cancer patients and control individuals by ELISA. ROC analyses (FIG. 3B, left panel) determined the cut off value of CEA for NSCLC detection to be 2.5 ng/ml (with a sensitivity of 42.7% ( 70/164 NSCLC; 4 of 24 (16.7%) stage I, 3 of 20 (15%) stage II-IIIA, and 63 of 120 (52.5%) stage IIIB-IV tumors) and a specificity of 97.7%), and also determined the cut-off value of CYFRA 21-1 to be 2.0 pg/ml, with a sensitivity of 39.0% ( 64/164 NSCLC; 1 of 24 (4.2%) stage I, 3 of 20 (15%) stage II-IIIA, and 60 of 120 (50%) stage IIIB-IV tumors) and a specificity of 96.2% (Table 4). The sum of the area under the ROC curve for serum Nectin-4 value was larger than that for serum CEA or CYFRA21-1, suggesting better specificity and likelihood for Nectin-4 as diagnostic biomarker for NSCLC.

TABLE 5
Assosciation between serum Nectin-positivity and NSCLC patient'
characteristics (n = 164)

| | | Serum | Serum | P-value |
|---|---|---|---|---|
| | | Nectin-4 | Nectin-4 | positive |
| | Total | positive | absent | vs |
| | n = 164 | n = 88 | n = 76 | absent |
| Gender | | | | |
| Male | 122 | 69 | 53 | 0.215 |
| Female | 42 | 19 | 23 | |
| Age (years) | | | | |
| <65 | 74 | 34 | 40 | 0.0845 |
| >=65 | 90 | 54 | 36 | |
| Histological type | | | | |
| ADC | 123 | 66 | 57 | >0.9999 |
| SCC | 41 | 22 | 19 | |
| pT factor | | | | |
| T1 | 35 | 11 | 24 | 0.0039* |
| T2-T4 | 129 | 77 | 52 | |
| pN factor | | | | |
| N0 | 50 | 21 | 29 | 0.0612 |
| N1-N3 | 114 | 67 | 47 | |
| Smoking | | | | |
| non-smoker | 50 | 24 | 26 | 0.3959 |
| smoker | 114 | 64 | 50 | |

ADC, adenocarcinoma:
SCC, squamous carcinoma
*P < 0.05 (Fisher's exact test)

TABLE 6
Association between serum Nectin-4-positivity and normal
volunteer' characteristics (n = 131)

| | | Serum | Serum | |
|---|---|---|---|---|
| | | Nectin-4 | Nectin-4 | P-value |
| | Total | positive | absent | positive |
| | n = 131 | n = 3 | n = 128 | vs absent |
| Gender | | | | |
| Male | 88 | 2 | 86 | >0.9999** |
| Female | 43 | 1 | 42 | |
| Age (years) | | | | |
| <65 | 116 | 2 | 114 | 0.3078** |
| >=65 | 15 | 1 | 14 | |
| Smoking | | | | |
| pack-years | 16.4 ± 20.8 | 21.7 ± 33.3 | 15.3 ± 20.6 | 0.6589⁺ |
| (±1SD) | | | | |
| current smoker | 36 | 1 | 35 | 0.6004**^{, ++} |
| former smoker | 27 | 1 | 26 | |
| never smoker | 68 | 1 | 67 | |

**Fisher's exact test
⁺Mann-Whitney U test
⁺⁺current & former vs never

TABLE 7
Association between serum Nectin-4-positivity and COPD patients' characteristics (n = 86)

| | | Serum Nectin-4 | Serum Nectin-4 | P-value |
|---|---|---|---|---|
| | Total | positive | absent | positive |
| | n = 86 | n = 7 | n = 79 | vs absent |
| Gender | | | | |
| Male | 76 | 7 | 69 | >0.9999** |
| Female | 10 | 0 | 10 | |
| Age (years) | | | | |
| <65 | 27 | 2 | 25 | >0.9999** |
| >=65 | 59 | 5 | 54 | |
| Smoking | | | | |
| pack-years(±1SD) | 66.7 ± 44.4 | 69.3 ± 60.0 | 66.5 ± 43.2 | 0.8572⁺ |
| current smoker | 16 | 1 | 15 | >0.9999** |
| former smoker | 70 | 6 | 64 | |
| Respiratory function(±1SD) | | | | |
| FEV 1.0(I) | 1409.4 ± 576.7 | 1486.7 ± 597.2 | 1433.9 ± 589.3 | 0.8341⁺ |
| FEV 1/FVC % | 52.7 ± 15.2 | 56.6 ± 12.1 | 51.4 ± 13.8 | 0.383⁺ |
| FEV1(% pred) | 46.9 ± 19.2 | 47.6 ± 19.4 | 46.4 ± 18.3 | 0.8879⁺ |

**Fisher's exact test
⁺Mann-Whitney U test

Next, the feasibility of using serum Nectin-4 level as a tumor detection biomarker in combination with CEA and CYFRA 21-1 was evaluated. Measuring both Nectin-4 and CEA in serum can improve overall sensitivity for detection of lung ADC to 65.0% (for diagnosing ADC, the sensitivity of CEA alone is 42.3% and that of Nectin-4 is 54.5%. False-positive results for either of the two tumor-markers among 131 healthy volunteers (control group) amounted to 4.6% ( 6/131), while the false-positive rates for CEA and Nectin-4 in the same control group were 2.3% ( 3/131) each. On the other hand, Nectin-4 and CYFRA21-1 in serum can improve overall sensitivity for detection of lung SCC to 68.3% (for diagnosing SCC, the sensitivity of CYFRA21-1 alone is 53.7% and that of Nectin-4 is 51.2%). False-positive results for either of the two tumor-markers among 131 healthy volunteers (control group) amounted to 6.1% ( 8/131), while the false-positive rates for CYFRA21-1 and Nectin-4 in the same control group were 3.8% ( 5/131) and 2.3% ( 3/131) each.

ELISA experiments were then performed using paired preoperative and postoperative (two months after the surgery) serum samples from NSCLC patients to monitor the levels of serum Nectin-4 in the same patients. The concentration of serum Nectin-4 was reduced after surgical resection of primary tumors (FIG. 3B, right panel). The serum levels of Nectin-4 were further compared with the expression levels of Nectin-4 in primary tumors of the same set of 12 NSCLC cases whose serum had been collected before surgery (six patients with Nectin-4-positive tumors and six with Nectin-4-negative tumors). The serum levels of Nectin-4 showed good correlation with the expression levels of Nectin-4 in primary tumors (FIG. 3C). The results establish that serum Nectin-4 was secreted from lung tumor.

### Example 5

Association of serum Nectin-4 positivity with poor clinical outcomes for NSCLC patients.

Since strong Nectin-4 protein expression in primary tumor was associated with poor prognosis for NSCLC patients, the biological importance of serum Nectin-4 levels for lung cancer patients was next determined using serum samples from NSCLC patients with precise follow-up record, whose clinicopathological background affecting prognosis was mostly identical. The serum Nectin-4 levels were examined by ELISA for diagnosis in 88 patients with advanced NSCLC (stage IIIB-IV) who were newly diagnosed and previously untreated, and Nectin-4 positivity was correlated to clinical outcomes after the 1st-line standard chemotherapy with two drugs combination (Schiller J H, et al. N Engl J Med 2002; 346:92-8). The median survival time of serum Nectin-4 positive patients with advanced NSCLC was shorter than that of patients with serum Nectin-4 negative (Median Survival Times 242 versus 584 days, P=0.0042; FIG. 3D). Univariate analysis indicated that serum Nectin-4 positivity (positive versus negative; P=0.0056), poor performance status (PS 2-4 versus 0-1; P<0.0001), and advanced clinical stage (stage IV versus IIIB; P=0.0183) were significantly associated with poor prognosis for newly diagnosed advanced NSCLC patients (Table 8). Multivariate analysis confirmed that serum Nectin-4 positivity (P=0.0159) and poor performance status (P=0.0006) were independent prognostic factors for advanced NSCLC patients. The results independently support the high specificity and the utility of serum Nectin-4 as a biomarker for detection of cancer at an early stage and for predicting the early progression of the disease.

TABLE 8
Cox's proportional hazards model analysis of prognostic factors in patients with NSCLCs

| Variables | Hazards ratio | 95% Cl | Unfavorable/Favorable | P-value |
|---|---|---|---|---|
| Univariate analysis | | | | |
| Serum Nectin-4 | 2.606 | 1.318-5.127 | positive/negative | 0.0056* |
| Age (years) | 1.255 | 0.707-2.227 | >=65/<65 | 0.4385 |
| Gender | 1.153 | 0.573-2.320 | Male/Female | 0.6908 |
| Histological type | 1.044 | 0.542-2.014 | non-ADC/ADC | 0.8969 |
| Performance status | 3.668 | 1.955-6.883 | PS 2-4/PS 0-1 | <0.0001* |
| stage | 2.41 | 1.161-5.005 | IV/IIIB | 0.0183* |
| Multivariate analysis | | | | |
| Serum Nectin-4 | 2.334 | 1.172-4.650 | positive/negative | 0.0159* |
| Performance status | 3.087 | 1.622-5.873 | PS 2-4/PS 0-1 | 0.0006* |
| stage | 2.096 | 0.994-4.424 | IV/IIIB | 0.052 |

ADC, adenocarcinoma.
*P < 0.05

To further confirm the association between serum Nectin-4 positivity and poor prognosis, the serum Nectin-4 levels were examined by ELISA for before curative surgery in additional 95 patients with stage I NSCLC and those at diagnosis in additional 62 patients with advanced NSCLC (stage IIIB-IV) who were newly diagnosed and previously untreated, and whose clinicopathological background affecting prognosis was mostly identical, and found the correlation of serum Nectin-4 positivity with clinical outcomes. The median survival time of serum Nectin-4 positive patients with stage I NSCLC after curative surgery or advanced NSCLC treated with an identical protocol of chemotherapy was shorter than those of patients with serum Nectin-4 negative (P=0.0219 and 0.0269, respectively by log-rank test; FIG. 3E, top and bottom panels). Univariate analysis was also applied to evaluate associations between patient prognosis and other factors including age, gender, histological type, disease stage, smoking history and serum Nectin-4 positivity. Univariate analysis indicated that only serum Nectin-4 positive was significantly associated with poor prognosis for early stage NSCLC patients after surgery (P=0.0301; Table 9). In advanced NSCLCs, univariate analysis indicated that serum Nectin-4 positivity (positive versus negative; P=0.036), poor performance status (PS 2-4 versus 0-1; P=0.0004), and advanced clinical stage (stage IV versus IIIB; P=0.0183) were significantly associated with poor prognosis for newly diagnosed advanced NSCLC patients (Table 10). Multivariate analysis confirmed that both serum Nectin-4 positivity and poor performance status (P=0.0006) were independent prognostic factors for advanced NSCLC patients (P=0.0394 and 0.0004, respectively; Table 10). These results independently support the high specificity and the utility of serum Nectin-4 as a biomarker for detection of cancer at an early stage and for predicting the early progression of the disease.

TABLE 9
Cox's proportional hazards model analysis of prognostic factors
in early NSCLC (Stage I) patients treated with surgery

| | Hazards | | Unfavorable/ | |
|---|---|---|---|---|
| Variables | ratio | 95% Cl | Favorable | P-value |
| Univariate | | | | |
| analysis | | | | |
| Serum Nectin-4 | 3.209 | 1.119-9.203 | positive/ | 0.0301* |
| | | | negative | |
| Age (years) | 1.486 | 0.466-4.738 | >=65/<65 | 0.5033 |
| Gender | 1.36 | 0.472-3.922 | Male/Female | 0.5688 |
| Histological type | 1.144 | 0.256-5.118 | ADC/non-ADC | 0.8602 |
| pT factor | 1.092 | 0.366-3.258 | T2/T1 | 0.8751 |
| Smoking | 1.388 | 0.481-4.011 | smoker/ | 0.3689 |
| | | | non-smoker | |

ADC, adenocarcinoma,
*P < 0.05

TABLE 10
Cox's proportional hazards model analysis of prognostic factors in advanced NSCLC (Staple IIIB-IV)
patients treated with chemotherapy

| Variables | Hazards ratio | 95% Cl | Unfavorable/Favorable | P-value |
|---|---|---|---|---|
| Univariate analysis | | | | |
| Serum Nectin-4 | 3.145 | 1.078-9.176 | positive/negative | 0.0360* |
| Age (years) | 1.167 | 0.527-2.585 | >=65/<65 | 0.7026 |
| Gender | 1.09 | 0.454-2.613 | Male/Female | 0.8475 |
| Histological type | 2.026 | 0.794-5.170 | non-ADC/ADC | 0.1395 |
| Performance status | 15.852 | 3.467-72.488 | PS 2/PS 0-1 | 0.0004* |
| Stage | 2.836 | 0.848-9.488 | IV/IIIB | 0.0906 |
| Smoking | 1.147 | 0.493-2.669 | smoker/non-smoker | 0.7497 |
| Multivariate analysis | | | | |
| Serum Nectin-4 | 3.139 | 1.074-9.173 | positive/negative | 0.0394* |
| Performance status | 15.765 | 1.074-72.173 | PS 2-4/PS 0-1 | 0.0004* |
| Univariate analysis | | | | |
| Serum Nectin-4 | 3.145 | 1.078-9.176 | positive/negative | 0.0360* |
| Age (years) | 1.167 | 0.527-2.585 | >=65/<65 | 0.7026 |
| Gender | 1.090 | 0.454-2.613 | Male/Female | 0.8475 |
| Histological type | 2.026 | 0.794-5.170 | non-ADC/ADC | 0.1395 |
| Performance status | 15.852 | 3.467-72.488 | PS 2/PS 0-1 | 0.0004* |
| Stage | 2.836 | 0.848-9.488 | IV/IIIB | 0.0906 |
| Smoking | 1.147 | 0.493-2.669 | smoker/non-smoker | 0.7497 |
| Multivariate analysis | | | | |
| Nectin-4 | 3.139 | 1.074-9.173 | Serum Nectin-4 (+)/(−) | 0.0394* |
| Performance status | 15.765 | 1.074-72.173 | PS 2/PS 0-1 | 0.0004* |

ADC, adenocarcinoma,
*P < 0.05

### Example 6

### Effect of Nectin-4 siRNAs on the Growth of NSCLC Cells

To assess whether up-regulation of Nectin-4 plays a significant role in the growth and/or survival of lung-cancer cells, siRNAs for Nectin-4 were transfected to lung cancer cell lines NCI-H2170 and NCI-H358 that overexpressed endogenous Nectin-4. The levels of Nectin-4 expression in the cells transfected with siRNA against Nectin-4 (si-Nectin-4-#1 and -#2) were significantly reduced compared to cells transfected with any of the two control siRNAs (FIG. 4A, top panels). In accordance with the suppressive effect of the si-Nectin-4-#1, and -#2 on Nectin-4 expression, colony numbers and cell viability measured by 3-(4,5-dimethylthiazol-2-yl)-2,5-diphenyltetrazolium bromide (MTT) assays were significantly reduced (FIG. 4A, middle and bottom panels), but no such effects were observed by two control siRNAs.

To further examine the effect of Nectin-4 on the growth of lung cancer cells, transfected plasmids were designed to express Nectin-4 or mock plasmids into PC-14 cells that did not express endogenous Nectin-4, and established two independent PC-14 cell lines overexpressing exogenous Nectin-4 (PC-14-Nectin-4-#A and -#B) and two control cells (PC-14-Mock#A and -#B). MTT assay revealed that growth of the two PC-14-Nectin-4 cells was promoted at a significant degree compared to control PC-14-Mock cells (FIG. 4E). To investigate a potential role of Nectin-4 in vivo tumor growth, either PC-14-Nectin-4-#B cells or PC-14-Mock-#B cells subcutaneously were transplanted into BALB/cAJcl-nu/nu mice. During 20 days observation, all 3 mice that were individually transplanted with PC-14-Nectin-4-#B cells showed significantly more rapid tumor growth compared with 3 independent mice transplanted with PC-14-Mock-#B cells (FIG. 4F). These findings imply an in vivo and in vitro growth promoting effect of Nectin-4.

### Example 7

### Enhancement of Cellular Invasion by Overexpression of Nectin-4

As the immunohistochemical analysis on tissue microarray had indicated that NSCLC patients with Nectin-4-strong positive tumors showed shorter survival period than those with Nectin-4-negative or weak positive tumors, a possible role of Nectin-4 in cellular migration and invasion was examined by Matrigel assays using mammalian COS-7 and NIH-3T3 cells. As shown in FIG. 4B, cells transfected with myc/His-tagged Nectin-4-expressing plasmids showed significant invasive activity through Matrigel compared with cells transfected with mock vector.

Next, the effect of Nectin-4 on the cell morphology was examined by transfecting myc/His-tagged Nectin-4-expressing plasmids into COS-7 and NIH-3T3 cells. Immunocytochemical analysis using anti-myc antibody for exogenous Nectin-4 and phalloidin for F-actin clearly detected that the membrane protrusions, which were strongly stained with phalloidin, was significantly increased after transfection of Nectin-4 (FIG. 4C, top and bottom panels). No such effect was observed in the cells transfected with mock vector. Interestingly, exogenous Nectin-4 was strongly stained and partly colocalized with F-actin at these protrusions. These results suggest that overexpression of Nectin-4 induced lamellipodia formation in these cells.

### Example 8

### Activation of Rac1 by Overexpression of Nectin-4

The extension of protrusions, such as lamellipodia, is essential for cell motility, and the formation of lamellipodia requires activation of small GTPases Rac1 (Takai Y, et al. Physiol Rev 2001; 81:153-208). Therefore, it was examined whether Rac1 activity could be involved in Nectin-4-dependent cell motility. Like other small GTPases, Rac1 regulates molecular events by cycling between an inactive GDP-bound form and an active GTP-bound form. In its active (GTP-bound) state, Rac1 binds specifically to the p21-binding domain (PBD) of p21-activated protein kinase (PAK) to control downstream signaling cascades. Based on this mechanism, a possible activation of Rac1 was investigate by Nectin-4 overexpression, using lysate from COS-7 and NIH-3T3 cells transfected with Nectin-4-expressing plasmids or mock plasmids, which were mixed with PAK-PBD agarose beads to affinity-precipitate the protein complex containing GTP-Rac1 (active form). The levels of GTP-Rac1 were elevated in COS-7 and NIH-3T3 cells transfected with Nectin-4-expressing plasmids, compared to the cells transfected with mock vector (FIG. 4D). These results indicate that Nectin-4 could enhance the lamellipodia formation and cellular invasion possibly through activation of Rac1.

Discussion

Nectin-4 protein was overexpressed in the majority of NSCLCs, but scarcely expressed in normal tissues except placenta. Strong Nectin-4 expression was associated with shorter survival periods. Suppression of Nectin-4 expression with siRNA effectively suppressed growth of lung cancer cells. In addition, Nectin-4 expression increased the lamellipodia formation and the invasive ability of mammalian cells by activating Rac1. Although detailed functional association between Nectin-4 transactivation and lung carcinogenesis remains to be clarified, the combined results establish that Nectin-4 contributes to highly malignant phenotype of tumors through Rac1 signaling, and that targeting Nectin-4 pathway is useful for developing new types of therapeutic drugs such as sequence-specific gene silencing by nucleic acid drugs, monoclonal antibodies, and cancer vaccines that are expected to have a powerful biological activity against cancer with a minimal risk of adverse events.

According to the present invention, the original ELISA system was established by the two mouse monoclonal antibodies to measure serum levels of Nectin-4 and found that serum Nectin-4 levels were significantly higher in lung cancer patients than in healthy volunteers. This is a great advantage to develop practical and standardized diagnostic kits in the clinic. The ectodomain shedding of Nectin-4 may exert some signals important for cancer progression. Importantly, serum Nectin-4 values measured by our fully validated ELISA system showed higher sensitivity and specificity (53.7% and 97.7%, respectively) than conventional serum tumor markers for NSCLC (CEA or CYFRA21-1) that are being used in clinical practice (Table 4). Furthermore, serum Nectin-4 in patients with operable stages of NSCLC also showed higher sensitivity (25% in stage I; 50% in stage II-IIIA) than serum CEA (16.7% in stage I; 15% in stage II-IIIA) or CYFRA21-1 (4.2% in stage I; 15% in stage II-IIIA). An assay combining Nectin-4 with either of the markers (Nectin-4+CEA or Nectin-4+CYFRA21-1) increased the sensitivity to about 65% to 68% for NSCLC, significantly higher than that of CEA or CYFRA21-1 alone, whereas 4.6% to 6.1% of healthy volunteers were falsely diagnosed as positive. Furthermore, the presence of serum Nectin-4 was significantly associated with shorter survival periods for NSCLC patients. The data presented here demonstrate the clinical usefulness of Nectin-4 as a serologic biomarker for NSCLC that could be widely used in clinical practice, such as early detection of cancer, prediction of the malignant potential of tumor, and monitoring the disease control condition after any anticancer treatment. Finally, activation of Nectin-4 was observed in more than half of a series of other types of cancers such as bladder and cervical carcinomas, indicating its diagnostic and therapeutic application to a wide-range of tumors.

In summary, it has been shown that activation of Nectin-4 is an essential contributor to the growth and invasive activity in cancer cells. Moreover, Nectin-4 is a useful target for the development of therapeutic approaches such as molecular-targeted drugs and immunotherapy to any types of cancers over-expressing this molecule.

### INDUSTRIAL APPLICABILITY

As demonstrated herein, cell growth is suppressed by double-stranded molecules that specifically target the Nectin-4 gene. Thus, these novel double-stranded molecules are useful as anti-cancer pharmaceuticals. For example, agents that block the expression of Nectin-4 protein and/or prevent its activity find therapeutic utility as anti-cancer agents, particularly anti-cancer agents for the treatment of lung cancer, more particularly for the treatment of NSCLC.

The expression of Nectin-4 is markedly elevated in lung cancer, as compared to normal organs. Accordingly, these genes can be conveniently used as diagnostic markers of lung cancer and the proteins encoded thereby find utility in diagnostic assays of lung cancer.

Furthermore, the methods described herein are also useful in diagnosis of lung cancer, including non-small cell lung cancers (NSCLCs), as well as the prediction of the poor prognosis of the patients with these diseases. Moreover, the present invention provides new therapeutic approaches for treating cancer including lung cancers.

In one aspect, the present invention relates to the discovery that Nectin-4 levels are elevated in the sera of lung-cancer patients as compared to that of normal controls. Accordingly, the Nectin-4 protein has utility as a diagnostic marker, particularly a serological marker for lung cancer. Using the serum level of Nectin-4 as an index, the present invention provides methods for diagnosing, as well as monitoring the progress of cancer treatment, in cancer patients. The prior art fails to provide a suitable serological marker for lung cancer. Novel serological marker Nectin-4 of the present invention may improve the sensitivity for detection of lung cancer. In addition, the combination of Nectin-4 and CEA or CYFRA contributes to increase the sensitivity for detecting lung cancer.

Furthermore, Nectin-4 polypeptide is a useful target for the development of anti-cancer pharmaceuticals. For example, agents that bind Nectin-4 or block the expression of Nectin-4 or prevent its activity, may find therapeutic utility as anti-cancer agents, particularly anti-cancer agents for the treatment of lung cancer.

All publications, databases, sequences, patents, and patent applications cited herein are hereby incorporated by reference.

While the invention has been described in detail and with reference to specific embodiments thereof, it will be apparent to one skilled in the art that various changes and modifications can be made therein without departing from the spirit and scope of the invention, the metes and bounds of which are set by the appended claims.

1. A method for diagnosing cancer relating to Nectin-4 over-expression, said method comprising the steps of:
(a) determining a level of Nectin-4 in a biological sample derived from a subject; and
(b) comparing the level of Nectin-4 determined in step (a) with that of a normal control, wherein a high level of Nectin-4 in the biological sample, as compared to the normal control, indicates that the subject suffers from cancer.

2. The method of claim 1, wherein the cancer is lung cancer, bladder cancer and cervical carcinoma.

3. The method of claim 1, wherein the biological sample is a blood sample.

4. The method of claim 3, wherein the blood sample is selected from the group consisting of whole blood, serum and plasma.

5. The method of claim 4, wherein the level of Nectin-4 is determined by detecting the Nectin-4 protein in the serum.

6. The method of claim 5, wherein the Nectin-4 protein is detected by an immunoassay.

7. The method of claim 6, wherein the immunoassay is an ELISA.

8. The method of claim 7, wherein the immunoassay is carried out using an antibody raised against the ectodomain of Nectin-4.

9. The method of claim 3, further comprising the steps of:
(c) determining a level of CEA in the blood sample from the same subject;
(d) comparing the level of CEA determined in step (c) with that of a normal control; and
(e) judging that the subject suffers from cancer when the level of Nectin-4 and/or the level of CEA are higher than the control levels.

10. The method of claim 3, further comprising the steps of:
(c) determining a level of CYFRA in the blood sample from the same subject;
(d) comparing the level of CYFRA determined in step (c) with that of a normal control; and
(e) judging that the subject suffers from cancer when the level of Nectin-4 and/or the level of CYFRA are higher than the control levels.

11. The method of claim 9, wherein the cancer is lung cancer.

12. The method of claim 11, wherein the lung cancer is adenocarcinoma or squamous-cell carcinoma.

13. A method for assessing or determining the prognosis of a patient with a cancer related to Nectin-4 over-expression, wherein the method comprises the steps of:
(a) determining the expression level of the Nectin-4 gene in a patient-derived biological sample;
(b) comparing the expression level in step (a) with a control level; and
(c) determining the prognosis of the patient based on the comparison of step (b).

14. The method of claim 13, wherein the cancer is selected from the group consisting of lung cancer, bladder cancer and cervical carcinoma.

15. The method of claim 14, wherein the control level is a good prognosis control level and an increase of the expression level as compared to the control level is determined as poor prognosis.

16. The method of claim 15, wherein the patient-derived biological sample is a blood sample, and the expression level of the Nectin-4 gene is determined as a level of Nectin-4 in the blood sample.

17. The method of claim 16, wherein the blood sample is selected from the group consisting of whole blood, serum and plasma.

18. The method of claim 17, wherein the level of Nectin-4 is determined by detecting the Nectin-4 protein in the serum.

19. The method of claim 18, wherein the Nectin-4 protein is detected by an immunoassay.

20. The method of claim 19, wherein the immunoassay is an ELISA.

21. The method of claim 20, wherein the immunoassay is carried out using an antibody raised against the ectodomain of Nectin-4.

22. A kit for diagnosing cancer, or assessing or determining the prognosis of a patient with a cancer, wherein the cancer relates to Nectin-4 over-expression, wherein the kit comprises a reagent selected from the group consisting of:
(a) a reagent for detecting Nectin-4 mRNA;
(b) a reagent for detecting the protein encoded by the Nectin-4 gene; and
(c) a reagent for detecting the biological activity of the Nectin-4 protein.

23. The kit of claim 22, wherein the cancer is selected from the group consisting of lung cancer, bladder cancer and cervical carcinoma.

24. The kit of claim 22 for assessing or determining the prognosis of a patient with cancer, wherein the kit further comprises good prognosis control sample.

25. The kit of claim 22, wherein the kit further comprises a poor prognosis control sample.

26. The kit of claim 22, wherein the regent is an immunoassay reagent for detecting the protein encoded by the Nectin-4 gene.

27. The kit of claim 26, wherein the immunoassay reagent comprises an antibody raised against the ectodomain of Nectin-4.

28. The kit of claim 22, wherein the kit further comprises an immunoassay reagent for determining a level of CEA and/or CYFRA in a blood sample.

29. The kit of claim 28, wherein the kit further comprises a positive control sample for CEA and/or CYFRA.

30.-43. (canceled)

44. The method of claim 10, wherein the cancer is lung cancer.

45. The method of claim 44, wherein the lung cancer is adenocarcinoma or squamous-cell carcinoma.


SEQUENCE LISTING (11 sequences)

SEQ ID NO: 1 - DNA, 2744 bp, Homo sapiens
gttgttggccacagcgtgggaagcagctctgggggagctcggagctcccgatcacggcttcttgggggtagctacggctgggtgtgtagaacggggccggggctggggctgggtcccctagtggagacccaagtgcgagaggcaagaactctgcagcttcctgccttctgggtcagttccttattcaagtctgcagccggctcccagggagatctcggtggaacttcagaaacgctgggcagtctgcctttcaaccatgcccctgtccctgggagccgagatgtgggggcctgaggcctggctgctgctgctgctactgctggcatcatttacaggccggtgccccgcgggtgagctggggacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctgacccaggcctgcctcccttccctaggcctggctccttctgttgacatgggagattttagctcatcttgggggcctccttaaacacccccatttcttgcggaagatgctccccatcccactgactgcttgacctttacctccaacccttctgttcatcgggagggctccaccaattgagtctctcccaccatgcatgcaggtcactgtgtgtgtgcatgtgtgcctgtgtgagtgttgactgactgtgtgtgtgtggaggggtgactgtccgtggaggggtgactgtgtccgtggtgtgtattatgctgtcatatcagagtcaagtgaactgtggtgtatgtgccacgggatttgagtggttgcgtgggcaacactgtcagggtttggcgtgtgtgtcatgtggctgtgtgtgacctctgcctgaaaaagcaggtattttctcagaccccagagcagtattaatgatgcagaggttggaggagagaggtggagactgtggctcagacccaggtgtgcgggcatagctggagctggaatctgcctccggtgtgagggaacctgtctcctaccacttcggagccatgggggcaagtgtgaagcagccagtccctgggtcagccagaggcttgaactgttacagaagccctctgccctctggtggcctctgggcctgctgcatgtacatattttctgtaaatatacatgcgccgggagcttcttgcaggaatactgctccgaatcacttttaatttttttcttttttttttcttgccctttccattagttgtattttttatttatttttatttttatttttttttagagatggagtctcactatgttgctcaggctggccttgaactcctgggctcaagcaatcctcctgcctcagcctccctagtagctgggactttaagtgtacaccactgtgcctgctttgaatcctttacgaagag

SEQ ID NO: 2 - protein, 510 aa, Homo sapiens
MPLSLGAEMWGPEAWLLLLLLLASFTGRCPAGELGTSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 3 - DNA, 20 bp, Artificial
  An artificially synthesized primer sequence for RT-PCR
gtcagccagaggcttgaact

SEQ ID NO: 4 - DNA, 20 bp, Artificial
  An artificially synthesized primer sequence for RT-PCR
ggattcaaagcaggcacagt

SEQ ID NO: 5 - DNA, 21 bp, Artificial
  An artificially synthesized primer sequence for RT-PCR
atcaagatcattgctcctcct

SEQ ID NO: 6 - DNA, 20 bp, Artificial
  An artificially synthesized primer sequence for RT-PCR
ctgcgcaagttaggttttgt

SEQ ID NO: 7 - DNA, 20 bp, Artificial
  An artificially synthesized primer sequence for preparing probe
cagggggttaattcctgtga

SEQ ID NO: 8 - DNA, 21 bp, Artificial
  An artificially synthesized primer sequence for preparing probe
caactaatggaaagggcaaga

SEQ ID NO: 9 - DNA, 19 bp, Artificial
  An artificially synthesized oligonucleotide for siRNA
cgtacgcggaatacttcga

SEQ ID NO: 10 - DNA, 19 bp, Artificial
  An artificially synthesized oligonucleotide for siRNA
acagttaccacgtctgagg

SEQ ID NO: 11 - DNA, 19 bp, Artificial
  An artificially synthesized oligonucleotide for siRNA
aatggttcatggcctgttt